(19) United States
(12) Patent Application Publication
(10) Pub. No.: US 2006/0223096 A1
(43) Pub. Date: Oct. 5, 2006

(54) Antigen binding molecules directed to MCSP and having increased Fc receptor binding affinity and effector function

(75) Inventors: Pablo Umana, Zurich (CH); Ekkehard Mossner, Kreuzlingen (CH)

Correspondence Address: STERNE, KESSLER, GOLDSTEIN & FOX PLLC, 1100 NEW YORK AVENUE, N.W., WASHINGTON, DC, 20005, US

(73) Assignee: GLYCART BIOTECHNOLOGY AG, Schlieren-Zurich (CH)

(21) Appl. No.: 11/388,204

(22) Filed: Mar. 24, 2006

Related U.S. Application Data
(60) Provisional application No. 60/665,079, filed on Mar. 25, 2005.

(51) Int. Cl. C12Q 1/68; G01N 33/574; C07H 21/04; C12P 21/06; C07K 14/82; C07K 16/30

(52) U.S. Cl. 435/6; 435/7.23; 435/69.1; 435/320.1; 435/325; 530/350; 530/388.22; 536/23.5

(57) ABSTRACT

The present invention relates to antigen binding molecules (ABMs). In particular embodiments, the present invention relates to recombinant monoclonal antibodies, including chimeric, primatized or humanized antibodies specific for human MCSP. In addition, the present invention relates to nucleic acid molecules encoding such ABMs, and vectors and host cells comprising such nucleic acid molecules. The invention further relates to methods for producing the ABMs of the invention, and to methods of using these ABMs in treatment of disease. In addition, the present invention relates to ABMs with modified glycosylation having improved therapeutic properties, including antibodies with increased Fc receptor binding and increased effector function.

### CROSS-REFERENCE TO RELATED APPLICATIONS

This application claims the benefit of U.S. Provisional Application No. 60/665,079, filed Mar. 25, 2005, the entire contents of which are hereby incorporated by reference.

### BACKGROUND OF THE INVENTION

1. Field of the Invention

The present invention relates to antigen binding molecules (ABMs). In particular embodiments, the present invention relates to recombinant monoclonal antibodies, including chimeric, primatized and humanized antibodies specific for the high molecular weight—melanoma-associated antigen (HMW-MAA), also known as the melanoma chondroitin sulfate proteoglycan (MCSP). In addition, the present invention relates to nucleic acid molecules encoding such ABMs, and vectors and host cells comprising such nucleic acid molecules. The invention further relates to methods for producing the ABMs of the invention, and to methods of using these ABMs in treatment of disease. In addition, the present invention relates to ABMs with modified glycosylation having improved therapeutic properties, including antibodies with increased Fc receptor binding and increased effector function.

2. Background Art

Melanoma Associated Antigens

Malignant melanoma is the most common type of fatal skin cancer in humans, and its incidence is estimated to be increasing at a rate of 5% per year. Campoli et al., Crit. Rev. Immunol. 24(4):267-296 (December 2004). The mortality rate has also increased over the last decade despite advances in diagnosis and therapy. While early stage melanoma is highly treatable, advanced stage melanoma is frequently resistant to conventional therapeutic regimens. The limitations of conventional therapies have stimulated research into novel strategies for treating patients with malignant melanoma. Much of the research has focused on immunotherapies.

Most of the human melanoma immunotherapy regimes being developed focus on melanoma-associated antigens (MAA). The preferred MAAs for immunotherapy are antigens that are expressed in a large percentage of melanomas but have restricted distribution in normal tissues. One such antigen is the melanoma chondroitin sulfate proteoglycan (MCSP), also referred to as the high molecular weight-melanoma-associated antigen (HMW-MAA). Pluschke et al., Proc. Natl. Acad. Sci. USA 93:9710-9715 (1996); Yang et al., J. Cell Biol. 165(6):881-891 (June 2004). MCSP is a highly glycosylated integral membrane chondroitin sulfate proteoglycan consisting of an N-linked 280 kDa glycoprotein component and a 450-kDa chondroitin sulfate proteoglycan component expressed on the cell membrane. Ross et al., Arch. Biochem. Biophys. 225:370-383 (1983). Proteoglycans are proteins covalently linked to glycoaminoglycans (GAG). Both the 280-kDa component and the 450-kDa component of MCSP contain the same core protein. Ross et al., Arch. Biochem. Biophys. 225:370-383 (1983); Bumol et al., J. Biol. Chem. 259:12733-12741 (1984).

The cDNA encoding the full-length MCSP core protein has been identified and the amino acid sequence deduced. Pluschke et al., Proc. Natl. Acad. Sci. USA 93:9710-9715 (1996); Yang et al., J. Cell Biol. 165(6):881-891 (June 2004) (the contents of each of which are herein incorporated by reference in their entirety). The MCSP sequence has been deposited and assigned the following accession numbers: GenBank Accession No. MIM:601172 (gene); GI:1617313, GI:21536290, GI:34148710, and GI:47419929 (mRNA); GI:1617314, GI:4503099, GI:34148711, and GI:47419930 (protein). The core protein, consisting of 2322 amino acids, contains 3 major domains: a large extracellular domain, a hydrophobic transmembrane region, and a short cytoplasmic tail. Homology searches using the MCSP sequence indicate that homologues are expressed in other animal species. Specifically, the rat and mouse homologues of MCSP are known as NG2 and AN2, respectively. Each shares substantial amino acid sequence identity with MCSP and has a similar expression profile. Stallcup et al., J. Neurocytol 31:423-435 (2002); Schneider et al., J. Neurosci. 21:920-933 (2001).

It was originally thought that MCSP had restricted tissue distribution as it was initially detected only in cells of melanocyte lineage, as well as cells within hair follicles, the basal cell layer of the skin epidermis, endothelial cells, and pericytes. Ferrone et al., Pharmacol. Ther. 57:259-290 (1993); Schlingemann et al., Am. J. Pathol. 136:1393-1405 (1990). More recently, however, it has been determined that MCSP is more broadly distributed in a number of normal and transformed cells. In particular, MCSP is found in almost all basal cells of the epidermis.

The link between MCSP and melanoma is well-established. MCSP is differentially expressed in melanoma cells, and was found to be expressed in more than 90% of benign nevi and melanoma lesions analyzed. Campoli et al., Crit. Rev. Immunol. 24(4):267-296 (December 2004). Moreover, MCSP expression has not been found to vary between primary and metastatic lesions in all types of melanoma. Kageshita et al., Int. J. Cancer 56:370-374 (1994). MCSP has also been found to be expressed in tumors of nonmelanocytic origin, including basal cell carcinoma, various tumors of neural crest origin, and in breast carcinomas. Kageshita et al., J. Invest. Dermatol. 85:535-537 (1985); Chekenya et al., Int. J. Dev. Neurosci. 17:421-435 (1999); Chekenya et al., J. Neurocytol. 31:507-521 (2002); Shoshan et al., Proc. Nat. Acad. Sci. USA 96:10361-10366 (1999); Godal et al., Br. J. Cancer 53:839-841 (1986); Dell'Erba et al., Anticancer Res. 21:925-930 (2001).

Substantial evidence indicates that MCSP is differentially involved in influencing the malignant behavior of melanoma cells. It is well known that both the GAG constituent and the core protein of proteoglycans generally are responsible for binding several different ligands including, but not limited to, adhesion molecules, chemokines, cytokines, extracellular matrix (ECM) components, and growth factors. Bernfield et al., Ann. Rev. Biochem. 68:729-777 (1999). With respect to MCSP specifically, studies have demonstrated that MCSP-specific antibodies can inhibit melanoma cell attachment to capillary endothelium and spreading on various ECM components, including collagen and collagen-fibronectin complexes. Harper et al., J. Natl. Cancer Inst. 71:259-263 (1983); de Vries et al., Int. J. Cancer 38:465-473 (1986); Iida et al., Cancer Res. 55:2177-2185 (1995); Burg et al., J. Cell. Physiol. 177:299-312 (1998). Other studies have shown that MCSP expression is restricted to cell surface microspike domains on migrating melanoma cells. Garrigues et al., J. Cell Biol. 103:1699-1710 (1986). Microspike domains are actin-rich structures that are important for the formation of adhesive contacts with ECM components in migrating cells. Collectively, the evidence indicates that MCSP promotes the formation of initial adhesive contacts at the leading edge of migrating cells.

Additional evidence indicates that MCSP also modulates melanoma cell migration through a second mechanism involving the initiation of intracellular signaling events. In particular, both MCSP and NG2 have been shown to trigger signal transduction pathways through activation of Rho GTPase family proteins. Stallcup et al., J. Neurocytol. 31:423-435 (2002); Eisenmann et al., Nat. Cell Biol. 1:507-513 (1999). Other studies indicate that MCSP expression leads to enhanced activation of focal adhesion kinase (FAK) by an integrin-dependent mechanism and to enhanced activation of extracellular signal-regulated kinase (ERK) by an independent mechanism. Yang et al., J. Cell Biol. 165:881-891 (June 2004).

MCSP is also implicated in melanoma cell proliferation. Specifically, melanoma cells transfected to express MCSP or NG2 exhibit enhanced proliferation rates in vitro and increased growth rates in vivo. These effects are inhibited by anti-MCSP or anti-NG2 monoclonal antibodies.

Substantial evidence indicates that MCSP plays a key role in angiogenesis and melanoma cell invasion. First, MCSP is expressed at high levels in both “activated” pericytes and pericytes in tumor angiogenic vasculature. Ruiter et al., Behring Inst. Mitt. 92:258-272 (1993). Pericytes are known to be associated with endothelial cells developing vasculature and it is thought that they participate in the regulation of angiogenesis by controlling endothelial cell proliferation and invasion. Witmer et al., J. Histochem. Cytochem 52(1):39-52 (2004); Erber et al., FASEB 18:338-340 (2004); Darland et al., Dev. Biol. 264:275-288 (2003). Second, MCSP and NG2 are widely expressed by angiogenic blood vessels in normally developing tissues. Chekenya et al., FASEB 16:586-588 (2002); Ruiter et al., Behring Inst. Mitt. 92:258-272 (1993).

The high level of expression of MCSP on melanoma cells, along with its restricted distribution in normal tissues and the availability of MCSP specific mAbs make MCSP a logical marker for melanoma lesions and a candidate target for immunotherapy. Indeed, a number of murine monoclonal antibodies to MCSP have been developed. Campoli et al., Crit. Rev. Immunol. 24(4):267-296 (2004). Although murine anti-MCSP mAbs have been shown to control tumor growth in animal models, only minor clinical responses were observed in clinical trials with these antibodies. What benefits are seen are generally attributed to the ability of the anti-MCSP antibody to influence the biology of melanoma cells and not to antibody-mediated immunologic mechanisms. Accordingly, most of the MCSP targeted immunotherapies utilize anti-idiotypic antibodies that mimic the MCSP epitope.

Unconjugated monoclonal antibodies (mAbs) can be useful medicines for the treatment of cancer, as demonstrated by the U.S. Food and Drug Administration's approval of Trastuzumab (Herceptin™; Genentech Inc,) for the treatment of advanced breast cancer (Grillo-Lopez, A.-J., et al., Semin. Oncol. 26:66-73 (1999); Goldenberg, M. M., Clin. Ther. 21:309-18 (1999)), Rituximab (Rituxan™; IDEC Pharmaceuticals (now Biogen IDEC), San Diego, Calif. and Cambridge, Mass., and Genentech Inc., San Francisco, Calif.), for the treatment of CD20 positive B-cell, low-grade or follicular Non-Hodgkin's lymphoma, Gemtuzumab (Mylotarg™, Celltech/Wyeth-Ayerst) for the treatment of relapsed acute myeloid leukemia, and Alemtuzumab (CAMPATH™, Millenium Pharmaceuticals/Schering AG) for the treatment of B cell chronic lymphocytic leukemia. The success of these products relies not only on their efficacy but also on their outstanding safety profiles (Grillo-Lopez, A.-J., et al., Semin. Oncol. 26:66-73 (1999); Goldenberg, M. M., Clin. Ther. 21:309-18 (1999)). In spite of the achievements of these drugs, there is currently a large interest in obtaining higher specific antibody activity than what is typically afforded by unconjugated mAb therapy.

The results of a number of studies suggest that Fc-receptor-dependent mechanisms contribute substantially to the action of cytotoxic antibodies against tumors and indicate that an optimal antibody against tumors would bind preferentially to activation Fc receptors and minimally to the inhibitory partner FcγRIIB. (Clynes, R. A., et al., Nature Medicine 6(4):443-446 (2000); Kalergis, A. M., and Ravetch, J. V., J. Exp. Med. 195(12):1653-1659 (June 2002). For example, the results of at least one study suggest that the FcγRIIIa receptor in particular is strongly associated with the efficacy of antibody therapy. (Cartron, G., et al., Blood 99(3):754-757 (February 2002)). That study showed that patients homozygous for a polymorphism in FcγRIIIa have a better response to Rituximab than heterozygous patients. The authors concluded that the superior response was due to better in vivo binding of the antibody to FcγRIIIa, which resulted in better ADCC activity against lymphoma cells. (Cartron, G., et al., Blood 99(3):754-757 (February 2002)).

Various immunotherapy strategies that target MCSP have been reported. Early immunotherapies used MCSP as a target in antibody-based passive immunotherapy in advanced melanoma patients. In general, anti-MCSP antibodies were administered to patients alone or conjugated to toxins. Schroffet al., Cancer Res. 45:879-885 (1985); Spitler et al., Cancer Res. 47:1717-1723 (1987); Bumol et al., Proc. Nat. Acad. Sci. USA 80:529-533 (1983). Although such antibodies exhibited tumor inhibition in animal models, only minor clinical responses were observed in a few patients. Matsui et al., Jap. J. Cancer Res. 76:119-123 (1985); Morgan et al., J. Natl. Cancer Inst. 78:1101-1106 (1987); Ghose et al., Cancer Immunol. Immunother. 34:90-96 (1991). More recently, improved therapeutic responses have been observed with single-chain Fv immunoconjugates and with anti-idiotypic antibodies monoclonal antibodies that target MCSP. Wang et al., Proc. Natl. Acad. Sci. USA 96:1627-1632 (1999); Kang et al., Clin. Cancer Res. 6:4921-4931 (2000); Mittelman et al., Proc. Nat. Acad. Sci. USA 89:466-470 (1992); U.S. Pat. No. 5,270,202; U.S. Pat. No. 5,780,029; U.S. Pat. No. 5,866,124. However, these immunotherapies have drawbacks. Specifically, anti-idiotypic antibodies are not useful in targeting MCSP expressing cells. Also, scFv constructs lack Fc regions and therefore cannot alone induce lysis of MCSP positive target cells.

Most of the anti-MCSP monoclonal antibodies that have been developed are murine. They include mAb 149.53, mAb 225.28; mAb 763.74; and mAb 9.2.27. Campoli et al., Crit. Rev. Immunol 24(4):267-296 (2004). To date, only a few anti-MCSP antibodies from human sources have been isolated. Wang et al., Proc. Nat. Acad. Sci. 96:1627-1632 (1999). It is believed that to date none of the murine (or any other nonhuman) anti-MCSP antibodies have been humanized. A potential problem with the use of murine antibodies in therapeutic treatments is that non-human monoclonal antibodies can be recognized by the human host as a foreign protein; therefore, repeated injections of such foreign antibodies can lead to the induction of immune responses leading to harmful hypersensitivity reactions. For murine-based monoclonal antibodies, this is often referred to as a Human Anti-Mouse Antibody response, or “HAMA” response, or a Human Anti-Rat Antibody, or “HARA” response. Additionally, these “foreign” antibodies can be attacked by the immune system of the host such that they are, in effect, neutralized before they reach their target site. Furthermore, non-human monoclonal antibodies (e.g., murine monoclonal antibodies) typically lack human effector functionality, i.e., they are unable to, inter alia, mediate complement dependent lysis or lyse human target cells through antibody dependent cellular toxicity or Fc-receptor mediated phagocytosis.

Chimeric antibodies comprising portions of antibodies from two or more different species (e.g., mouse and human) have been developed as an alternative to “conjugated” antibodies.

Antibody Glycosylation

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. (Jenkins et al., Nature Biotechnol. 14:975-81 (1996)).

Mammalian cells are the preferred hosts for production of therapeutic glycoproteins, due to their capability to glycosylate proteins in the most compatible form for human application. (Cumming et al., Glycobiology 1:115-30 (1991); Jenkins et al., Nature Biotechnol. 14:975-81 (1996)). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum-free media, and permit the development of safe and reproducible bioprocesses. Other commonly used animal cells include baby hamster kidney (BHK) cells, NS0- and SP2/0-mouse myeloma cells. More recently, production from transgenic animals has also been tested. (Jenkins et al., Nature Biotechnol. 14:975-81 (1996)).

All antibodies contain carbohydrate structures at conserved positions in the heavy chain constant regions, with each isotype possessing a distinct array of N-linked carbohydrate structures, which variably affect protein assembly, secretion or functional activity. (Wright, A., and Morrison, S. L., Trends Biotech. 15:26-32 (1997)). The structure of the attached N-linked carbohydrate varies considerably, depending on the degree of processing, and can include high-mannose, multiply-branched as well as biantennary complex oligosaccharides. (Wright, A., and Morrison, S. L., Trends Biotech. 15:26-32 (1997)). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R. et al., Glycobiology 5(8):813-22 (1995)).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umafia, P. et al., Nature Biotechnol. 17:176-180 (1999) and U.S. Pat. No. 6,602,684, the contents of which are hereby incorporated by reference in their entirety. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5:813-822 (1995); Jefferis, R., et al., Immunol Rev. 163:59-76 (1998); Wright, A. and Morrison, S. L., Trends Biotechnol. 15:26-32 (1997)).

Umafia et al. showed previously that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of an anti-neuroblastoma chimeric monoclonal antibody (chCE7) produced by the engineered CHO cells. (See Umafia, P. et al., Nature Biotechnol. 1 7:176-180 (1999); and International Publication No. WO 99/54342, the entire contents of which are hereby incorporated by reference). The antibody chCE7 belongs to a large class of unconjugated mAbs which have high tumor affinity and specificity, but have too little potency to be clinically useful when produced in standard industrial cell lines lacking the GnTIII enzyme (Umana, P., et al., Nature Biotechnol. 17:176-180 (1999)). That study was the first to show that large increases of ADCC activity could be obtained by engineering the antibody-producing cells to express GnTIII, which also led to an increase in the proportion of constant region (Fc)-associated, bisected oligosaccharides, including bisected, nonfucosylated oligosaccharides, above the levels found in naturally-occurring antibodies.

There remains a need for enhanced therapeutic approaches targeting MCSP for the treatment of cell proliferation disorders in mammals, including, but not limited to, humans, wherein such disorders are characterized by MCSP expression, particularly abnormal expression (e.g., overexpression) including, but not limited to, melanomas, gliomas, lobular breast cancer, and also tumors that induce neovasculature.

### BRIEF SUMMARY OF THE INVENTION

Recognizing the tremendous therapeutic potential of antigen binding molecules (ABMs) that have the binding specificity of the murine 225.28S antibody and that have been glycoengineered to enhance Fc receptor binding affinity and effector function, the present inventors developed a method for producing such ABMs. Inter alia, this method involves producing recombinant, chimeric (including humanized) antibodies or chimeric fragments thereof. The efficacy of these ABMs is further enhanced by engineering the glycosylation profile of the antibody Fc region.

Thus, in one embodiment, the present invention is directed to an isolated polynucleotide comprising: (A) a sequence selected from the group consisting of: SEQ ID NO:61; SEQ ID NO:63; SEQ ID NO:65; SEQ ID NO:67; SEQ ID NO:69; SEQ ID NO:71; SEQ ID NO:73; SEQ ID NO:75; and (B) a sequence selected from the group consisting of: SEQ ID NO:77; SEQ ID NO:79; SEQ ID NO:81; SEQ ID NO:83; SEQ ID NO:85; SEQ ID NO:87; SEQ ID NO:89; SEQ ID NO:91; and SEQ ID NO:93; and (C) SEQ ID NO:95. Preferably, the isolated polynucleotide encodes a fusion protein.

In another embodiment, the invention is directed to an isolated polynucleotide comprising: (A) a sequence selected from the group consisting of: SEQ ID NO:97; SEQ ID NO:99; and SEQ ID NO:101; and (B) SEQ ID NO:103 or SEQ ID NO:105; and (C) SEQ ID NO:107. Preferably, the isolated polynucleotide encodes a fusion protein.

In a further embodiment, the present invention relates to an isolated polynucleotide comprising a sequence selected from the group consisting of: SEQ ID No:3; SEQ ID No:5; SEQ ID No:7; SEQ ID No:9; SEQ ID No:11; SEQ ID No:13; SEQ ID No:15; SEQ ID No:17; SEQ ID No:19; SEQ ID No:21; and SEQ ID No:23. The invention also relates to an isolated polynucleotide comprising a sequence selected from the group consisting of SEQ ID No:29, SEQ ID No:31, and SEQ ID No:33; SEQ ID NO:35; SEQ ID NO:37; SEQ ID NO:39; SEQ ID NO:41; SEQ ID NO:43; SEQ ID NO:45; SEQ ID NO:47; SEQ ID NO:49; SEQ ID NO:51. Preferably, the isolated polynucleotide encodes a fusion protein.

A further embodiment of the invention relates to an isolated polynucleotide comprising: (A) a sequence encoding a polypeptide having a sequence selected from the group consisting of SEQ ID No:2; SEQ ID No:4; SEQ ID No:6; SEQ ID No:8; SEQ ID No:10; SEQ ID No:12; SEQ ID No:14; SEQ ID No: 16; SEQ ID No: 18; SEQ ID No:20; SEQ ID No:22; SEQ ID No:24; and (B) a sequence encoding a polypeptide having a sequence selected from the group consisting of: SEQ ID NO:28 SEQ ID NO:30, SEQ ID NO:32, SEQ ID NO:34 and SEQ ID NO:36; SEQ ID NO:38; SEQ ID NO:40; SEQ ID NO:42; SEQ ID NO:44; SEQ ID NO:46; SEQ ID NO:48; SEQ ID NO:50; SEQ ID NO:52.

In another embodiment, the invention relates to an isolated polynucleotide comprising a sequence having at least 80%, alternatively at least 85%, alternatively at least 90%, alternatively at least 95%, alternatively at least 99%, identity to a sequence selected from the group consisting of: SEQ ID No:1; SEQ ID No:3; SEQ ID No:5; SEQ ID No:7; SEQ ID No:9; SEQ ID No:11; SEQ ID No: 13; SEQ ID No:15; SEQ ID No:17; SEQ ID No: 19; SEQ ID No:21; and SEQ ID No:23, wherein said isolated polynucleotide encodes a fusion polypeptide. Such isolated polynucleotides may further comprise a nucleotide sequence encoding a human antibody light or heavy chain constant region.

In yet another embodiment, the invention also relates to an isolated polynucleotide comprising a sequence having at least 80%, alternatively at least 85%, alternatively at least 90%, alternatively at least 95%, alternatively at least 99%, identity to a sequence selected from the group consisting of: SEQ ID NO:27, SEQ ID NO:29, SEQ ID NO:31; SEQ ID NO:33; SEQ ID NO:35; SEQ ID NO:37; SEQ ID NO:39; SEQ ID NO:41; SEQ ID NO:43; SEQ ID NO:45; SEQ ID NO:47; SEQ ID NO:49; and SEQ ID NO:51, wherein said isolated polynucleotide encodes a fusion polypeptide. Such isolated polynucleotides may (further comprise a nucleotide sequence encoding a human antibody light or heavy chain constant region.

The present invention also relates to an isolated polynucleotide comprising: (A) a sequence encoding a polypeptide having a sequence selected from the group consisting of SEQ ID No:2; SEQ ID No:4; SEQ ID No:6; SEQ ID No:8; SEQ ID No:10; SEQ ID No:12; SEQ ID No:14; SEQ ID No:16; SEQ ID No: 18; SEQ ID No:20; SEQ ID No:22; and SEQ ID No:24; and (B) a sequence encoding a polypeptide having the sequence of an antibody Fc region, or a fragment thereof, from a species other than a murine species. Alternatively, the invention encompasses an isolated polynucleotide comprising: (A) a sequence encoding a polypeptide having sequence selected from the group consisting of: SEQ ID No:28, SEQ ID NO:30, SEQ ID NO:32; SEQ ID NO:34; SEQ ID NO:36; SEQ ID NO:38; SEQ ID NO:40; SEQ ID NO:42; SEQ ID NO:46; SEQ ID NO:48; and SEQ ID NO:52; and (B) a sequence encoding a polypeptide having the sequence of an antibody light chain constant domain, or a fragment thereof, from a species other than mouse.

The invention is further directed to an isolated polynucleotide encoding a polypeptide having a sequence selected from the group consisting of SEQ ID No:4; SEQ ID No:6; SEQ ID No:8; SEQ ID No:l0; SEQ ID No:12; SEQ ID No:14; SEQ ID No:16; SEQ ID No:18; SEQ ID No:20; SEQ ID No:22; and SEQ ID No:24. In another embodiment, the invention is directed to n isolated polynucleotide encoding a polypeptide having selected from the group consisting of SEQ ID NO:30, SEQ ID NO:32; SEQ ID NO:34; SEQ ID NO:36; SEQ ID NO:38; SEQ ID NO:40; SEQ ID NO:42; SEQ ID NO:46; SEQ ID NO:48; and SEQ ID NO:52.

The present invention also encompasses an expression vector comprising one or more of the polynucleotides of the invention set forth above. Preferably, the expression vector encodes at least the light or heavy chain of an antibody. In one embodiment, the expression vector encodes both the light and heavy chain of an antibody. The vector can be a polycistronic vector.

The present invention further relates to a host cell comprising one or more expression vectors of the present invention or one or more polynucleotides of the invention. In one embodiment, the host comprises an isolated polynucleotide comprising a sequence having at least 80%, alternatively at least 85%, alternatively at least 90%, alternatively at least 95%, alternatively at least 99%, identity to a sequence selected from the group consisting of: SEQ ID No:1; SEQ ID No:3; SEQ ID No:5; SEQ ID No:7; SEQ ID No:9; SEQ ID No:11; SEQ ID No:13; SEQ IDNo:15; SEQ IDNo:17; SEQ IDNo:19; SEQ IDNo:21; and SEQ ID No:23, and further comprises a second polynucleotide comprising a sequence encoding the variable region of an antibody light chain. In yet another embodiment, host cell of the invention comprises an isolated polynucleotide comprising a sequence having at least 80%, alternatively at least 85%, alternatively at least 90%, alternatively at least 95%, alternatively at least 99%, identity to a sequence selected from the group consisting of: SEQ ID NO:27, SEQ ID NO:29, SEQ ID NO:31; SEQ ID NO:33; SEQ. ID NO:35; SEQ ID NO:37; SEQ ID NO:39; SEQ ID NO:41; SEQ ID NO:43; SEQ ID NO:45; SEQ ID NO:47; SEQ ID NO:49; and SEQ ID NO:5 1, and further comprises a second polynucleotide comprising a sequence encoding the variable region of an antibody heavy chain.

In other embodiments, the present invention is directed to a fusion polypeptide comprising a sequence selected from the group consisting of SEQ ID No:2; SEQ ID No:4; SEQ ID No:6; SEQ ID No:8; SEQ ID No:10; SEQ ID No:12; SEQ ID No:14; SEQ ID No:16; SEQ ID No:18; SEQ ID No:20; SEQ ID No:22; and SEQ ID No:24, or a variant thereof.

The invention is also directed to a fusion polypeptide comprising a sequence selected from the group consisting of: SEQ ID NO:28; SEQ ID NO:30, SEQ ID NO:32; SEQ ID NO:34; SEQ ID NO:36; SEQ ID NO:38; SEQ ID NO:40; SEQ ID NO:42; SEQ ID NO:46; SEQ ID NO:48; and SEQ ID NO:52. or a variant thereof.

The present invention also relates to antigen binding molecules. Thus, in one embodiment, the invention is directed to an antigen binding molecule comprising one or more fusion polypeptides of the invention. Preferably, the antigen binding molecule selectively binds to human MCSP. In one preferred embodiment, the antigen binding molecule is an antibody. A humanized antibody is a particularly preferred antigen binding molecule. Alternatively, the antibody can be primatized. In other embodiments, the antigen binding molecule of the invention comprises an antibody fragment having an antibody Fc region or a region equivalent to the Fc region of an antibody. In still other embodiments, the antigen binding molecule of the invention is a scFv, diabody, triabody, tetrabody, Fab or Fab₂ fragment. In a preferred embodiment, the antigen binding molecule is a recombinant antibody, for example, a humanized, recombinant antibody. The recombinant antibody will generally comprise a human Fc region, preferably a human IgG Fc region.

In another embodiment, the present invention relates to an antigen binding molecule as discussed above that has been glycoengineered to have an Fc region with modified oligosaccharides. In one embodiment, the Fc region has been modified to have a reduced number of fucose residues as compared to the nonglycoengineered antigen binding molecule. In another embodiment, the Fc region has an increased proportion of bisected oligosaccharides as compared to the nonglycoengineered antigen binding molecule. In yet another embodiment, the bisected oligosaccharides are predominantly bisected complex. In another embodiment, the glycoengineered antigen binding molecules of the invention have an increased proportion of bisected, nonfucosylated oligosaccharides in the Fc region of said antigen binding molecule as compared to the nonglycoengineered antigen binding molecule. Alternatively, the antigen binding molecules of the invention may have an increased ratio of GlcNAc residues to fucose residues in the Fc region compared to the nonglycoengineered antigen binding molecule.

In one embodiment, the bisected, nonfucosylated oligosaccharides are predominantly hybrid form. Alternatively, the bisected, nonfucosylated oligosaccharides are predominantly complex type. In certain embodiments, at least 20%, at least 30%, at least 35% or at least 40%, of the oligosaccharides in the Fc region are bisected, nonfucosylated. In other embodiments, at least 50%, or at least 60%, or at least 70%, or at least 80%, or at least 90%, or at least 95%, of the oligosaccharides in the Fc region are bisected. In yet another embodiment, at least 50%, alternatively at least 60%, alternatively at least 70%, alternatively at least 75%, of the oligosaccharides in the Fc region are nonfucosylated.

The present invention is also directed to a method of producing an antigen binding molecule capable of competing with the murine 225.28S monoclonal antibody for binding to human MCSP, said method comprising: a) culturing the host cell of the invention under conditions allowing the expression of a polynucleotide encoding said antigen binding molecule; and b) recovering said antigen binding molecule. In one embodiment, the antigen binding molecule is an antibody, such as a humanized antibody.

The invention is further directed to a pharmaceutical composition comprising an antigen binding molecule of the invention and a pharmaceutically acceptable carrier. The pharmaceutical composition may optionally comprise an adjuvant.

The invention is still further directed to a method for identifying cells expressing MCSP in a sample or a subject comprising administering to said sample or subject an antigen binding molecule of the invention. In one embodiment, the identification is for diagnostic purposes. In another embodiment, the identification is for therapeutic purposes, such as treatment of a disease or disorder. Thus, in certain aspects, the invention is directed to a method of treating an MCSP-mediated cell proliferation disorder in a subject in need thereof comprising administering a therapeutically effective amount of a pharmaceutical composition of the invention to said subject. Preferably, the subject is a human. In one embodiment, the treatment comprises blocking MCSP-mediated interactions selected from the group consisting of: MCSP ligand binding, melanoma cell adhesion, pericyte activation, chemotactic responses to fibronectin, cell spreading on ECM proteins, FAK signal transduction and ERK signal transduction. In another embodiment, the disease or disorder treated is selected from the group consisting of: melanoma, glioma, lobular breast cancer, acute leukemia, or a solid tumor inducing neovascularization of blood vessels. In yet another embodiment, the treatment comprises killing of MCSP-expressing cells, preferably cells overexpressing MCSP.

The present invention is further directed to a host cell glycoengineered to express at least one nucleic acid encoding a first polypeptide having β(1,4)-N-acetylglucosaminyltransferase III activity in an amount sufficient to modify the oligosaccharides in the Fc region of a second polypeptide produced by said host cell, wherein said second polypeptide is an antigen binding molecule of the invention. In one embodiment, the host cell further expresses a polypeptide having mannosidase II activity. In a specific embodiment, the first polypeptide further comprises the localization domain of a Golgi resident polypeptide. Preferably, the antigen binding molecule of the invention is an antibody or antibody fragment. In another embodiment, antigen binding molecule comprises the Fc region of a human IgG or a region equivalent to the Fc region of a human IgG.

The antigen binding molecules produced by the host cells of the invention exhibit increased Fc receptor binding affinity and/or increased effector functions compared to the antigen binding molecule produced by the nonglycoengineered host cell.

In certain embodiments, the first polypeptide expressed by the host cell comprises the catalytic domain of β(1,4)-N-acetylglucosaminyltransferase III. In one embodiment, said first polypeptide further comprises the Golgi localization domain of a heterologous Golgi resident polypeptide, such as the localization domain of mannosidase II, the localization domain of β(1,2)-N-acetylglucosaminyltransferase I, the localization domain of β(1,2)-N-acetylglucosaminyltransferase II, the localization domain of mannosidase I, or the localization domain of α1-6 core fucosyltransferase.

The increased effector function exhibited by the antigen binding molecules produced by the host cells of the invention is one or more of increased Fc-mediated cellular cytotoxicity, increased binding to NK cells, increased binding to macrophages, increased binding to polymorphonuclear cells, increased binding to monocytes, increased direct signaling inducing apoptosis, increased dendritic cell maturation, or increased T cell priming.

The increased Fc receptor binding exhibited by the antigen binding molecules of the invention is, in one embodiment, increased binding to an Fcγ activating receptor, such as FcγRIII. In a specific embodiment, the increased binding is to the human FcγRIIIa receptor or a naturally occurring variant thereof.

In certain embodiments, the host cell of the invention is an HEK293-EBNA cell, a CHO cell, a BHK cell, a NSO cell, a SP2/0 cell, a YO myeloma cell, a P3X63 mouse myeloma cell, a PER cell, a PER.C6 cell or a hybridoma cell. In one embodiment, the host cell of the invention comprises at least one nucleic acid encoding a polypeptide having β(1,4)-N-acetylglucosaminyltransferase III activity that is operably linked to a constitutive promoter element. In another embodiment, the polypeptide having β(1,4)-N-acetylglucosaminyltransferase III activity that is expressed by the host cell is a fusion polypeptide.

The present invention also encompasses an isolated polynucleotide comprising at least one, alternatively at least two, alternatively at least three, complementarity determining region of the murine 225.28S monoclonal antibody, or a variant or truncated form thereof containing at least the specificity-determining residues for said complementarity determining region, wherein said isolated polynucleotide encodes a fusion polypeptide. Preferably, the complementarity determining region is selected from the group consisting of: SEQ ID NO:61; SEQ ID NO:63; SEQ ID NO:65; SEQ ID NO:67; SEQ ID NO:69; SEQ ID NO:71; SEQ ID NO:73; SEQ ID NO:75; SEQ ID NO:77; SEQ ID NO:79; SEQ ID NO:81; SEQ ID NO:83; SEQ ID NO:85; SEQ ID NO:87; SEQ ID NO:89; SEQ ID NO:91; SEQ ID NO:93; and SEQ ID NO:95. In another embodiment, the complementarity determining region is selected from the group consisting of: SEQ ID NO: 97, SEQ ID NO: 99, SEQ ID NO: 101; SEQ ID NO: 103; SEQ ID NO:105; SEQ ID NO: 107. Preferably, the fusion polypeptide encodes an antigen binding molecule of the invention.

In a specific embodiment, the CDRs comprise at least one sequence selected from the group consisting of: SEQ ID NO:61; SEQ ID NO:63; SEQ ID NO:65; SEQ ID NO:67; SEQ ID NO:69; SEQ ID NO:71; SEQ ID NO:73; SEQ ID NO:75; SEQ ID NO:77; SEQ ID NO:79; SEQ ID NO:81; SEQ ID NO:83; SEQ ID NO:85; SEQ ID NO:87; SEQ ID NO:89; SEQ ID NO:91; SEQ ID NO:93; and SEQ ID NO:95; and at least one sequence selected from the group consisting of: SEQ ID NO: 97, SEQ ID NO: 99, SEQ ID NO:101; SEQ ID NO: 103; SEQ ID NO:105; SEQ ID NO: 107, or variants or truncated forms of said sequences that contain at least the specificity-determining residues for each of said complementarity determining regions. The invention also encompasses the polypeptides encoded by such polynucleotides, as well as antigen binding molecules comprising such polypeptides.

The antigen binding molecules of the invention will, in some embodiments, comprise the variable region of an antibody light or heavy chain. In other embodiments, the ABM will be a chimeric or humanized, antibody.

The present invention is further directed to a method for producing an antigen binding molecule having modified oligosaccharides in a host cell, said method comprising: (a) culturing a host cell glycoengineered to express at least one nucleic acid encoding a polypeptide having β(1,4)-N-acetylglucosaminyltransferase III activity under conditions which permit the production of said antigen binding molecule, and which permit the modification of the oligosaccharides present on the Fc region of said antigen binding molecule; and (b) isolating said antigen binding molecule wherein said antigen binding molecule is capable of competing with the murine 225.28S monoclonal antibody for binding to MCSP and wherein said antigen binding molecule or fragment thereof is chimeric or humanized. In one method of the invention, the modified oligosaccharides have a reduced proportion of fucose residues as compared to the oligosaccharides of the nonglycoengineered antigen binding molecule. In certain embodiments, the modified oligosaccharides are predominantly hybrid form. In an alternative embodiment, the modified oligosaccharides are predominantly complex form. In another embodiment, at least 40%, at least 50%, at least 60%, at least 70%, at least 80%, or at least 90% of the modified oligosaccharides are bisected, nonfucosylated.

In other embodiments of the methods of the invention, the recombinant antibody or fragment thereof produced by said host cell has an increased proportion of bisected, nonfucosylated oligosaccharides in the Fc region of said polypeptide as compared to the antigen binding molecule produced by the nonglycoengineered cell. In one embodiment, the bisected, nonfucosylated oligosaccharides are predominantly hybrid form. In another embodiment, bisected, nonfucosylated oligosaccharides are predominantly complex form. In certain embodiments, at least 20%, at least 30%, at least 35%, or at least 40%, of the oligosaccharides in the Fc region of said polypeptide are bisected, nonfucosylated.

The antigen binding molecules produced by the methods ofthe invention will, in certain embodiments, have increased effector function and or increased Fc receptor binding affinity. In one embodiment, said antigen binding molecule is an antibody. The increased effector function can be one or more of increased Fc-mediated cellular cytotoxicity, increased binding to NK cells, increased binding to macrophages, increased binding to monocytes, increased binding to polymorphonuclear cells, direct signaling inducing apoptosis, increased dendritic cell maturation, or increased T cell priming. Preferably, the increased Fc receptor binding is increased binding to a Fc activating receptor, such as FcγRIIIa.

The present invention is also directed to an antigen binding molecule that is a fusion protein that includes a polypeptide having a sequence selected from the group consisting of: SEQ ID No:2; SEQ ID No:4; SEQ ID No:6; SEQ ID No:8; SEQ ID No:10; SEQ ID No:12; SEQ ID No:14; SEQ ID No:16; SEQ ID No:18; SEQ ID No:20; SEQ ID No:22; and SEQ ID No:24; and a region equivalent to the Fc region of an immunoglobulin and engineered to have increased effector function. In another embodiment, said antigen binding molecule is a fusion protein that includes a polypeptide having a sequence selected from the group consisting of SEQ ID NO:28, SEQ ID NO:30, SEQ ID NO:32, SEQ ID NO: 34, SEQ ID NO: 36; SEQ ID NO: 38, SEQ ID NO:40, SEQ ID NO: 42, SEQ ID NO: 44, SEQ ID NO:46, SEQ ID NO:48, SEQ ID NO:50, and SEQ ID N052, and a region equivalent to the Fc region of an immunoglobulin and engineered to have increased effector function. The invention is also directed to a pharmaceutical composition comprising such an antigen binding molecules and a pharmaceutically acceptable carrier.

The present invention is also directed to a method of inducing lysis of activated pericytes in tumor neovasculature in a subject in need thereof, comprising administering to said subject an antigen binding molecule of the invention or a pharmaceutical composition comprising same. Preferably, said subject is a human. In one embodiment, the said neovasculature is not melanoma neovasculature or glioblastoma neovasculature. In another embodiment, the antigen binding molecule is coadministered with another anti-angiogenic agent, such as an anti-VEGF-1 antibody.

### BRIEF DESCRIPTION OF THE FIGURES

FIG. 1 shows the binding activity of the three heavy chain constructs M-HHA, M-HHB, and M-HHC as well as the three light chain constructs M-KV1, M-KV2, and M-KV3. The humanized heavy chain constructs were coexpressed with murine light chain (mVL), and the humanized light chain constructs were coexpressed with the murine heavy chain (mVH). M-HHA and M-HHB more or less retained their binding properties when combined with murine VL. In contrast, M-HHC loses its binding potential significantly. M-KV1 and M-KV2 show strongly diminished binding activity compared to the murine counterpart, whereas M-KVC shows binding behavior similar to the murine light chain.

FIG. 2 shows the binding data of the “low-homology” constructs M-HLA, M-HLB, and M-HLC.

FIG. 3 shows the binding data of light chain constructs M-KV4, M-KV5, M-KV6, M-KV7, M-KV8 and M-KV9 when paired with the M-HHB heavy chain. M-KV4 showed increased affinity to antigen compared to the ch-225.28S antibody, while M-KV5 and M-KV6 lost functional properties and M-KV7 showed binding similar to ch-225.28S.

FIG. 4 shows results of antigen binding assay when heavy chain constructs M-HLE1, M-HLE2, M-HLF and M-HLG were paired with the light chain construct m-KV4. Constructs M-HLE1 and M-HLE2 showed some residual binding, while M-HLF showed almost no binding. M-HLG, on the other hand, showed higher affinity to antigen than the parental antibody ch-225.28S.

FIG. 5 shows binding of the light chain variant M-KV9 when combined with M-HHB. This construct showed good binding data.

FIG. 6 shows the comparison of different glycoforms of the humanized M-HLG/M-KV9 construct of the 225.28S antibody in antibody-mediated cell killing using human PBMC cells. Target cells are human A2058 cells, and one can see a strong increase in potency and efficacy of the glycoengineered construct compared to the wild-type antibody.

FIG. 7 shows the comparison of the antigen binding behavior of the light chain constructs M-KV10, M-KV11, and M-KV12, combined with the M-HLG heavy chain. These variants all show reduced binding compared to the M-KV9 light chain construct. Also shown in this figure is the M-HLD heavy chain paired with the M-KV9 light chain. M-HLD is the Tyr27Phe and Thr30Ser variant of the completely inactive construct M-HLC. Thus, these two mutations partially restore antigen binding activity. This indicates the importance of these two residues for the whole humanization process.

FIG. 8 shows the MALDI/TOF-MS profile of PNGaseF-released Fc-oligosaccharides of the non-glycoengineered M-HLG/M-KV9 G2 humanized IgG1 225.28S anti-human MCSP antibody. The two main peaks at 1485.5 and 1647.6 m/z both correspond to hybrid bisected fucosylated sugars.

FIG. 9 shows the MALDI/TOF-MS profile of PNGaseF-released Fc-oligosaccharides of the glycoengineered M-HLG/M-KV9 G2 humanized IgG1 225.28S anti-human MCSP antibody. Glycoengineering done by co-expression in host cells of antibody genes and genes encoding enzyme with β-1,4-N-acetylglucosaminyltransferase III (GnT-III) catalytic activity and encoding enzyme with Golgi oc-mannosidase II catalytic activity. The four main peaks at 1542.9, 1688.7, 1704.6, and 1850.5 all correspond to complex bisected sugars, which are present in their fucosylated as well as in their non-fucosylated form.

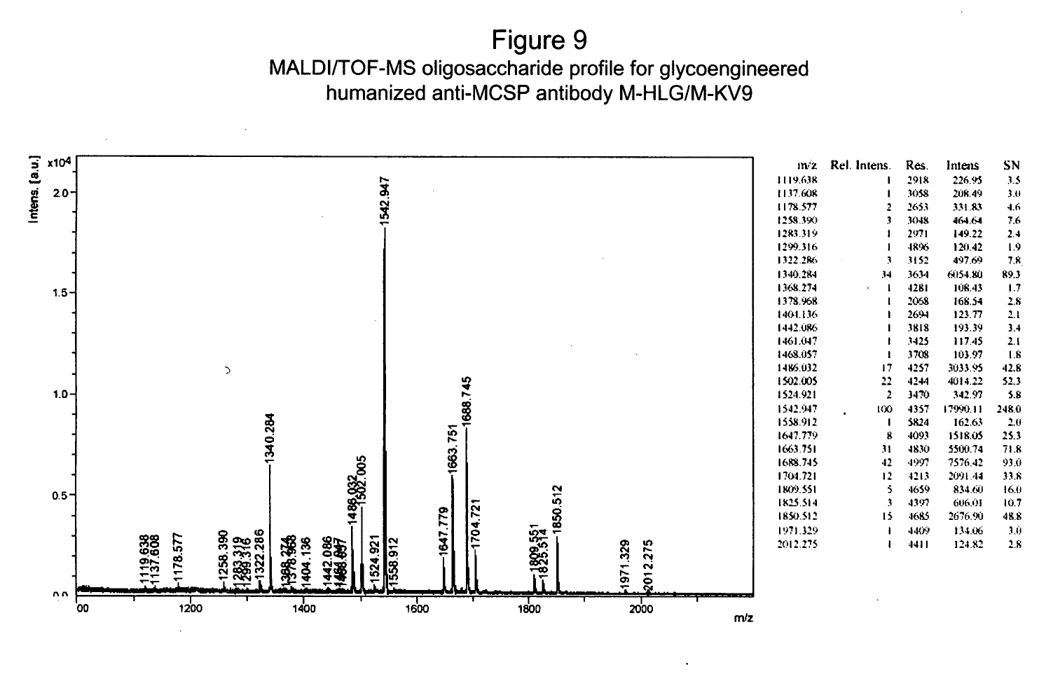

FIG. 10 shows a schematic drawing of the different N-linked oligosaccharides that can be affected by the glycoengineering via GnTIII and/or ManII coexpression.

FIG. 11 shows antibody dependent cellular cytotoxicity (ADCC) using the M-HLG/M-KV9 antibody, human smooth muscle cells (HuSMC) as targets, and human PBMC as effector cells. Effector/target ratio was 25/1, and the duration of the experiment was 4 h.

FIG. 12 shows antibody dependent cellular cytotoxicity (ADCC) using the M-HLG/M-KV9 antibody in its non-glycoengineered as well as in its glycoengineered form (G2), human glioblastoma cell line LN229 as targets, and human PBMC as effector cells. Effector/target ratio was 25/1, and the duration of the experiment was 4 h.

### DETAILED DESCRIPTION OF THE INVENTION

Terms are used herein as generally used in the art, unless otherwise defined as follows.

As used herein, the term antibody is intended to include whole antibody molecules, including monoclonal, polyclonal and multispecific (e.g., bispecific) antibodies, as well as antibody fragments having the Fc region and retaining binding specificity, and fusion proteins that include a region equivalent to the Fc region of an immunoglobulin and that retain binding specificity. Also encompassed are chimeric and humanized antibodies, as well as camelized and primatized antibodies.

As used herein, the term Fc region is intended to refer to a C-terminal region of a human IgG heavy chain. Although the boundaries of the Fc region of an IgG heavy chain might vary slightly, the human IgG heavy chain Fc region is usually defined to stretch from the amino acid residue at position Cys226 to the carboxyl-terminus.

As used herein, the term region equivalent to the Fc region of an immunoglobulin is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity. (See, e.g., Bowie, J. U. et al., Science 247:1306-10 (1990).

As used herein, the term MCSP refers to the human melanoma chondroitin sulfate proteoglycan (also known as the high molecular weight-melanoma-associated antigen (HMW-MAA)), as well as naturally-occurring isoforms and variants thereof. The MCSP sequences have been deposited and assigned the following accession numbers: GenBank Accession No. MIM:601172 (gene); GI: 1617313, GI:21536290, GI:34148710, and GI:47419929 (mRNA); GI:1617314, GI:4503099, GI:34148711, and GI:47419930 (protein)

As used herein, the term MCSP ligand refers to a polypeptide which binds to and/or activates MCSP. The term includes membrane-bound precursor forms of an MCSP ligand, as well as proteolytically processed soluble forms of an MCSP ligand.

As used herein, the term disease or disorder characterized by abnormal activation, expression, or production of MCSP or an MCSP ligand or disorder related to MCSP expression refers to a condition, which may or may not involve malignancy or cancer, where abnormal activation and/or production of MCSP and/or an MCSP ligand is occurring in cells or tissues of a subject having, or predisposed to, the disease or disorder.

As used herein, the terms overexpress, overexpressed, and overexpressing, as used in connection with cells expressing MCSP, refer to cells which have measurably higher levels of MCSP on the surface thereof compared to a normal cell of the same tissue type. Such overexpression may be caused by gene amplification or by increased transcription or translation. MCSP expression may be determined in a diagnostic or prognostic assay by evaluating levels of MCSP present on the surface of a cell (e.g. via an immunohistochemistry assay; immunofluorescence assay, immunoenzyme assay, ELISA, flow cytometry, radioimmunoassay, Western blot, ligand binding, kinase activity, etc.) (See generally, CELL BIOLOGY: A LABORATORY HANDBOOK, Celis, J., ed., Academic Press (2d ed., 1998); CURRENT PROTOCOLS IN PROTEIN SCIENCE, Coligan, J. E. et al., eds., John Wiley & Sons (1995-2003); see also, Sumitomo et al., Clin. Cancer Res. 10: 794-801 (2004) (describing Western blot, flow cytometry, and immunohistochemistry) the entire contents of which are herein incorporated by reference)). Alternatively, or additionally, one may measure levels of MCSP-encoding nucleic acid molecules in the cell, e.g. via fluorescent in situ hybridization, Southern blotting, or PCR techniques. The levels of MCSP in normal cells are compared to the levels of cells affected by a cell proliferation disorder (e.g., cancer) to determine if MCSP is overexpressed.

As used herein, the term antigen binding molecule or ABM refers in its broadest sense to a molecule that specifically binds an antigenic determinant. More specifically, an antigen binding molecule that binds MCSP is a molecule which specifically binds to MCSP as defined above. Preferably, the ABM is an antibody; however, single chain antibodies, single chain Fv molecules, Fab fragments, diabodies, triabodies, tetrabodies, and the like are also contemplated by the present invention.

By “specifically binds” or “binds with the same specificity” is meant that the binding is selective for the antigen and can be discriminated from unwanted or nonspecific interactions.

As used herein, the terms fusion and chimeric, when used in reference to polypeptides such as ABMs refer to polypeptides comprising amino acid sequences derived from two or more heterologous polypeptides, such as portions of antibodies from different species. For chimeric ABMs, for example, the non-antigen binding components may be derived from a wide variety of species, including primates such as chimpanzees and humans. The constant region of the chimeric ABM is most preferably substantially identical to the constant region of a natural human antibody; the variable region of the chimeric antibody is most preferably substantially identical to that of a recombinant anti-MCSP antibody having the amino acid sequence of the murine variable region. Humanized antibodies are a particularly preferred form of fusion or chimeric antibody.

As used herein, apolypeptide having “GnTIII activity” refers to polypeptides that are able to catalyze the addition of a N-acetylglucosamine (GlcNAc) residue in β-1-4 linkage to the C-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e., the candidate polypeptide will exhibit greater activity or not more than about 25-fold less and, preferably, not more than about tenfold less activity, and most preferably, not more than about three-fold less activity relative to the GnTIII.)

As used herein, the term variant (or analog) refers to a polypeptide differing from a specifically recited polypeptide of the invention by amino acid insertions, deletions, and substitutions, created using, e g., recombinant DNA techniques. Variants of the ABMs of the present invention include chimeric, primatized or humanized antigen binding molecules wherein one or several of the amino acid residues are modified by substitution, addition and/or deletion in such manner that does not substantially affect antigen (e.g., MCSP) binding affinity or antibody effector function. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequences.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to about 20 amino acids, more preferably about 1 to about 10 amino acids. The variation allowed may be experimentally determined by systematically making insertions, deletions, or substitutions of amino acids in a polypeptide molecule using recombinant DNA techniques and assaying the resulting recombinant variants for activity.

As used herein, the term humanized is used to refer to an antigen-binding molecule (ABM) derived from a non-human antigen-binding molecule, for example, a murine antibody, that retains or substantially retains the antigen-binding properties of the parent molecule but which is less immunogenic in humans. This may be achieved by various methods including (a) grafting only the non-human CDRs onto human framework and constant regions with or without retention of critical framework residues (e.g., those that are important for retaining good antigen binding affinity or antibody functions), or (b) transplanting the entire non-human variable domains, but “cloaking” them with a human-like section by replacement of surface residues. Such methods are disclosed in Jones et al., Nature 321:6069, 522-525 (1986); Morrison et al., Proc. Natl. Acad. Sci., 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994), all of which are incorporated by reference in their entirety herein. There are generally 3 complementarity determining regions, or CDRs, (CDR1, CDR2 and CDR3) in each of the heavy and light chain variable domains of an antibody, which are flanked by four framework subregions (i.e., FR1, FR2, FR3, and FR4): FR1-CDR1-FR2-CDR2-FR3-CDR3-FR4. A discussion of humanized antibodies can be found, inter alia, in U.S. Pat. No. 6,632,927, and in published U.S. Application No. 2003/0175269, both of which are incorporated herein by reference in their entirety.

Similarly, as used herein, the term primatized is used to refer to an antigen-binding molecule derived from a non-primate antigen-binding molecule, for example, a murine antibody, that retains or substantially retains the antigen-binding properties of the parent molecule but which is less immunogenic in primates.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR DEFINITIONS¹

| | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” refers to the CDRs as defined by Oxford Molecular's “AbM” antibody modeling software.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an ABM are according to the Kabat numbering system. The sequences of the sequence listing (i.e., SEQ ID NO:1 to SEQ ID NO:110) are not numbered according to the Kabat numbering system. However, as stated above, it is well within the ordinary skill of one in the art to determine the Kabat numbering scheme of any variable region sequence in the Sequence Listing based on the numbering of the sequences as presented therein.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

As a practical matter, whether any particular nucleic acid molecule or polypeptide is at least 80%, 85%, 90%, 95%, 96%, 97%, 98% or 99% identical to a nucleotide sequence or polypeptide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% ofthe amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N— or C-terminal deletions, not because of internal deletions, a manual correction must be made to the results. This is because the FASTDB program does not account for N— and C-terminal truncations of the subject sequence when calculating global percent identity. For subject sequences truncated at the N— and C-termini, relative to the query sequence, the percent identity is corrected by calculating the number of residues of the query sequence that are N— and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N— and C-termini of the subject sequence, which are not matched/aligned with the query sequence, are considered for the purposes of manually adjusting the percent identity score. That is, only query residue positions outside the farthest N— and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N— and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N— or C-termini of the subject sequence which are not matched/aligned with the query. In this case the percent identity calculated by FASTDB is not manually corrected. Once again, only residue positions outside the N— and C-terminal ends of the subject sequence, as displayed in the FASTDB alignment, which are not matched/aligned with the query sequence are manually corrected for. No other manual corrections are to be made for the purposes of the present invention.

As used herein, a nucleic acid that “hybridizes under stringent conditions” to a nucleic acid sequence of the invention, refers to a polynucleotide that hybridizes in an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

As used herein, the term Golgi localization domain refers to the amino acid sequence of a Golgi resident polypeptide which is responsible for anchoring the polypeptide in location within the Golgi complex. Generally, localization domains comprise amino terminal “tails” of an enzyme.

As used herein, the term effector function refers to those biological activities attributable to the Fc region (a native sequence Fc region or amino acid sequence variant Fc region) of an antibody. Examples of antibody effector functions include, but are not limited to, Fc receptor binding affinity, antibody-dependent cellular cytotoxicity (ADCC), antibody-dependent cellular phagocytosis (ADCP), cytokine secretion, immune-complex-mediated antigen uptake by antigen-presenting cells, down-regulation of cell surface receptors, etc.

As used herein, the terms engineer, engineered, engineering, glycoengineered and glycosylation engineering are considered to include any manipulation of the glycosylation pattern of a naturally occurring or recombinant polypeptide, such as an antigen binding molecule (ABM), or fragment thereof. Glycosylation engineering includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity.

As used herein, the term host cell covers any kind of cellular system which can be engineered to generate the polypeptides and antigen-binding molecules of the present invention. In one embodiment, the host cell is engineered to allow the production of an antigen binding molecule with modified glycoforms. In a preferred embodiment, the antigen binding molecule is an antibody, antibody fragment, or fusion protein. In certain embodiments, the host cells have been further manipulated to express increased levels of one or more polypeptides having GnTIII activity. In other embodiments, the host cells have been engineered to have eliminated, reduced or inhibited core α1,6-fucosyltransferase activity. The term “core α1,6-fucosyltransferase activity” encompasses both expression of the core α1,6-fucosyltransferase gene as well as interaction of the core α1,6-fucosyltransferase enzyme with its substrate. Host cells include cultured cells, e.g., mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

As used herein, the term Fc-mediated cellular cytotoxicity includes antibody-dependent cellular cytotoxicity and cellular cytotoxicity mediated by a soluble Fc-fusion protein containing a human Fc-region. It is an immune mechanism leading to the lysis of “antibody-targeted cells” by “human immune effector cells” , wherein:

The human immune effector cells are a population of leukocytes that display Fc receptors on their surface through which they bind to the Fc-region of antibodies or of Fc-fusion proteins and perform effector functions. Such a population may include, but is not limited to, peripheral blood mononuclear cells (PBMC) and/or natural killer (NK) cells.

The antibody-targeted cells are cells bound by the ABMs (e.g., antibodies or Fc-fusion proteins) of the invention. In general, the antibodies or Fc fusion-proteins bind to target cells via the protein part N-terminal to the Fc region.

As used herein, the term increased Fc-mediated cellular cytotoxicity is defined as either an increase in the number of “antibody-targeted cells” that are lysed in a given time, at a given concentration of antibody, or of Fc-fusion protein, in the medium surrounding the target cells, by the mechanism of Fc-mediated cellular cytotoxicity defined above, and/or a reduction in the concentration of antibody, or of Fc-fusion protein, in the medium surrounding the target cells, required to achieve the lysis of a given number of “antibody-targeted cells”, in a given time, by the mechanism of Fc-mediated cellular cytotoxicity. The increase in Fc-mediated cellular cytotoxicity is relative to the cellular cytotoxicity mediated by the same antibody, or Fc-fusion protein, produced by the same type of host cells, using the same standard production, purification, formulation and storage methods, which are known to those skilled in the art, but which have not been produced by host cells glycoengineered to express the glycosyltransferase GnTIII by the methods described herein.

By antibody having increased antibody dependent cellular cytotoxicity (ADCC) is meant an antibody, as that term is defined herein, having increased ADCC as determined by any suitable method known to those of ordinary skill in the art. One accepted in vitro ADCC assay is as follows:

1) the assay uses target cells that are known to express the target antigen recognized by the antigen-binding region of the antibody;

2) the assay uses human peripheral blood mononuclear cells (PBMCs), isolated from blood of a randomly chosen healthy donor, as effector cells;

3) the assay is carried out according to following protocol:
i) the PBMCs are isolated using standard density centrifugation procedures and are suspended at 5×10⁶ cells/ml in RPMI cell culture medium;
ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
iii) 100 microliters of the final target cell suspension above are transferred to each well of a 96-well microtiter plate;
iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
v) for the maximum release (MR) controls, 3 additional wells in the plate containing the labeled target cells, receive 50 microliters of a 2% (V/V) aqueous solution of non-ionic detergent (Nonidet, Sigma, St. Louis), instead of the antibody solution (point iv above);
vi) for the spontaneous release (SR) controls, 3 additional wells in the plate containing the labeled target cells, receive 50 microliters of RPMI cell culture medium instead of the antibody solution (point iv above);
vii) the 96-well microtiter plate is then centrifuged at 50×g for 1 minute and incubated for 1 hour at 4° C.;
viii) 50 microliters of the PBMC suspension (point i above) are added to each well to yield an effector:target cell ratio of 25:1 and the plates are placed in an incubator under 5% CO₂ atmosphere at 37° C. for 4 hours;
ix) the cell-free supernatant from each well is harvested and the experimentally released radioactivity (ER) is quantified using a gamma counter;
x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);

4) “increased ADCC′ is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

In one aspect, the present invention is related to antigen binding molecules having the same binding specificity as the murine 225.28S antibody, (i.e., binds to substantially the same epitope with substantially the same affinity) and to the discovery that their effector functions can be enhanced by altered glycosylation. In one embodiment, the antigen binding molecule is a chimeric antibody. In a preferred embodiment, the invention is directed to a chimeric antibody, or a fragment thereof, comprising at least one, alternatively at least two, alternatively at least three, alternatively at least four, alternatively at least five, or alternatively at least six of the CDRs of Tables 3 or 4 (SEQ ID NOs:62-108.) Specifically, in a preferred embodiment, the invention is directed to an isolated polynucleotide comprising: (a) a sequence selected from a group consisting of: SEQ ID NO:61 SEQ ID NO:63, SEQ ID NO:65, SEQ ID NO:67, SEQ ID NO:69 SEQ ID NO:71, SEQ ID NO:73, and SEQ ID NO:75; (b) a sequence selected from a group consisting of: SEQ ID NO:77, SEQ ID NO:79, SEQ ID NO:81, SEQ ID NO:83, SEQ ID NO:85, SEQ ID NO:87, SEQ ID NO:89, SEQ ID NO:91, and SEQ ID NO:93; and (c) SEQ ID NO:95. In another preferred embodiment, the invention is directed to an isolated polynucleotide comprising (a) a sequence selected from the group consisting of: SEQ ID NO:97; SEQ ID NO:99; and SEQ ID NO: 101; (b) a sequence selected from the group consisting of: SEQ ID NO:103 and SEQ ID NO:105; and (c) SEQ ID NO:107. In one embodiment, any of these polynucleotides encodes a fusion polypeptide.

In another embodiment, the antigen binding molecule comprises the V_{H} domain of the 225.28 antibody encoded by a sequence in Table 6 (SEQ ID NOS:1-23 odd), or a variant thereof; and a non-murine polypeptide. In another preferred embodiment, the invention is directed to an antigen binding molecule comprising the V_{L} domain of the rat antibody encoded by SEQ ID NOS:27-51 (odd) or a variant thereof; and a non-murine polypeptide.

In another aspect, the invention is directed to antigen binding molecules comprising one or more truncated CDRs of 225.28S. Such truncated CDRs will contain, at a minimum, the specificity-determining amino acid residues for the given CDR. By “specificity-determining residue” is meant those residues that are directly involved in the interaction with the antigen. In general, only about one-fifth to one-third of the residues in a given CDR participate in binding to antigen. The specificity-determining residues in a particular CDR can be identified by, for example, computation of interatomic contacts from three-dimensional modeling and determination of the sequence variability at a given residue position in accordance with the methods described in Padlan et al., FASEB J. 9(1):133-139 (1995), the contents of which are hereby incorporated by reference in their entirety.

Accordingly, the invention is also directed to an isolated polynucleotide comprising at least one complementarity determining region of the murine 225.28S antibody, or a variant or truncated form thereof containing at least the specificity-determining residues for said complementarity determining region, wherein said isolated polynucleotide encodes a fusion polypeptide. Preferably, such isolated polynucleotides encode a fusion polypeptide that is an antigen binding molecule. In one embodiment, the polynucleotide comprises two or three or four or five or six complementarity determining regions of the murine 225.28S antibody, or variants or truncated forms thereof containing at least the specificity-determining residues for each of said two or three or four or five or six complementarity determining regions. In one embodiment, the polynucleotide comprises at least one of the CDRs set forth in Tables 2 and 5, below). In another embodiment, the polynucleotide encodes the entire variable region of the light or heavy chain of a chimeric (e.g., humanized) antibody. The invention is further directed to the polypeptides encoded by such polynucleotides.

In another embodiment, the invention is directed to an antigen binding molecule comprising at least one, alternatively at least two, alternatively at least three, alternatively at least four, alternatively at least five, or alternatively at least six complementarity determining region of the murine 225.28S antibody, or a variant or truncated form thereof containing at least the specificity-determining residues for each said complementarity determining region, and comprising a sequence derived from a heterologous polypeptide. In one embodiment, the antigen binding molecule comprises three complementarity determining regions of the murine 225.28S antibody, or variants or truncated forms thereof containing at least the specificity-determining residues for each of said three complementarity determining regions. In one embodiment, the antigen binding molecule comprises at least one, alternatively at least two, alternatively at least three, alternatively at least four, alternatively at least five, or alternatively at least six of the CDRs set forth in Tables 3 and 4, below. In another aspect, the antigen binding molecule comprises the variable region of an antibody light or heavy chain. In one particularly useful embodiment, the antigen binding molecule is a chimeric, e.g., humanized, antibody. The invention is also directed to methods of making such antigen binding molecules, and the use of same in the treatment of disease, particularly cell proliferation disorders wherein MCSP is expressed, particularly wherein MCSP is abnormally expressed (e.g. overexpressed), compared to normal cells of the same tissue type. Such disorders include, but are not limited to, melanoma, glioma, lobular breast cancer, some acute leukemia, and all tumors inducing neovasculature. MCSP expression levels may be determined by methods known in the art and those described herein (e.g., via immunohistochemistry assay, immunofluorescence assay, immunoenzyme assay, ELISA, flow cytometry, radioimmunoassay, Western blot, ligand binding, kinase activity, etc.).

The invention is also directed to a method for targeting in vivo or in vitro cells expressing MCSP. Cells that express MCSP may be targeted for therapeutic purposes (e.g., to treat a disorder that is treatable by disruption of MCSP binding to a ligand, or by targeting MCSP-expressing cells for destruction by the immune system). In one embodiment, the present invention is directed to a method for targeting cells expressing MCSP in a subject comprising administering to the subject a composition comprising an ABM of the invention. Cells that express MCSP may also be targeted for diagnostic purposes (e.g., to determine if they are expressing MCSP, either normally or abnormally). Thus, the invention is also directed to methods for detecting the presence of MCSP or a cell expressing MCSP, either in vivo or in vitro. One method of detecting MCSP expression according to the present invention comprises contacting a sample to be tested, optionally with a control sample, with an ABM of the present invention, under conditions that allow for formation of a complex between the ABM and MCSP. The complex formation is then detected (e.g., by ELISA or other methods known in the art). When using a control sample with the test sample, any statistically significant difference in the formation of ABM-MCSP complexes when comparing the test and control samples is indicative of the presence of MCSP in the test sample.

TABLE 2

SEQ
ID

| | | |
|---|---|---|
| CDR | Nucleotide Sequence | NO |

| | | | |
|---|---|---|---|
| Heavy | Kabat | AATTACTGGATGAAC | 61 |
| Chain | | AGCTATTGGATGAGC | 63 |
| CDR1 | Chothia | GGATTCACTTTCAGTAAT | 65 |
| MCSP | | GGATACACATTCACCAAC | 67 |
| | | GGATTCACATTTAGCAGC | 69 |
| | AbM | GGATTCACTTTCAGTAATTACTGGATGAAC | 71 |
| | | GGATACACATTCACCAACTATTGGATGAAC | 73 |
| | | GGATTCACATTTAGCAGCTATTGGATGAGC | 75 |
| Heavy | Kabat | GAAATTAGATTGAAATCCAATAATTTTGGAAG | 77 |
| Chain | | ATATTATGCGGAGTCTGTGAAAGGG | |
| CDR2 | | GAGATCAGATTGAAATCCAATAACTTCGGAA | 79 |
| MCSP | | GATATTACGCTGCAAGCGTGAAGGGC | |
| | | AACATCAGATTGAAATCCAATAACTTCGGAA | 81 |
| | | GATATTACGCTGAGAGCGTGAAGGGC | |
| | | GAGATCAGATTGAAATCCAATAACTTCGGAA | 83 |
| | | GATATTACGCACAGAAGTTTCAGGGC | |
| | | GAAATCCGGTTGAAATCCAATAACTTCGGAA | 85 |
| | | GATACTACGCACAGAAGTTCCAGGAG | |
| | | GAGATCAGATTGAAATCCAATAACTTCGGAA | 87 |
| | | GATATTACGCTGCAAGCGTGAAGGGC | |
| | Chothia | AGATTGAAATCCAATAATTTTGGAAGATAT | 89 |
| | AbM | GAAATTAGATTGAAATCCAATAATTTTGGAAG | 91 |
| | | ATAT | |
| | | AACATCAGATTGAAATCCAATAACTTCGGAA | 93 |
| | | GATAT | |
| Heavy | Kabat | TATGGTAACTACGTTGGGCACTATTTTGACCA | 95 |
| Chain | Chothia | C | |
| CDR3 | AbM | | |
| MCSP | | | |

TABLE 3

| | | |
|---|---|---|
| CDR | Amino Acid Sequence | SEQ ID NO |

| | | | |
|---|---|---|---|
| Heavy | Kabat | NYWMN | 62 |
| Chain | | SYWMS | 64 |
| CDR1 | Chothia | GFTFSN | 66 |
| MCSP | | GYTFTN | 68 |
| | | GFTFSS | 70 |
| | AbM | GFTFSNYWMN | 72 |
| | | GYTFTNYWMN | 74 |
| | | GFTFSSYWMS | 76 |
| Heavy | Kabat | EIRLKSNNFGRYYAESVKG | 78 |
| Chain | | EIRLKSNNFGRYYAASVKG | 80 |
| CDR2 | | NIRLKSNNFGRYYAESVKG | 82 |
| MCSP | | EIRLKSNNFGRYYAQKFQG | 84 |
| | | EIRLKSNNFGRYYAQKFQE | 86 |
| | | EIRLKSNNFGRYYAASVKG | 88 |
| | Chothia | RLKSNNFGRY | 90 |
| | AbM | EIRLKSNNFGRY | 92 |
| | | NIRLKSNNFGRY | 94 |
| Heavy | Kabat | YGNYVGHYFDH | 96 |
| Chain | Chothia | | |
| CDR3 | AbM | | |
| MCSP | | | |

TABLE 4

| | | SEQ ID |
|---|---|---|
| CDR | Amino Acid Sequence | NO |
| Kabat Light Chain | KASQNVDTNVA | 98 |
| CDR1 (MCSP) | RASQNVDTNLA | 100 |
| | RASQNVDTNVA | 102 |
| Kabat Light Chain | SASYRYT | 104 |
| CDR2 (MCSP) | SASYLQS | 106 |
| Kabat Light Chain | QQYNSYPLT | 108 |
| CDR3 (MCSP) | | |

TABLE 5

SEQ
ID

| | | |
|---|---|---|
| CDR | Nucleotide Sequence | NO |

| | | |
|---|---|---|
| Kabat Light | AAGGCCAGTCAGAATGTGGATACTAATGTAGCG | 97 |
| Chain CDR1 | AGGGCCAGTCAGAATGTGGATACTAACTTAGCT | 99 |
| MCSP | AGGGCCAGTCAGAATGTGGATACTAACGTGGCT | 101 |
| Kabat Light | TCGGCATCCTACCGTTACACT | 103 |
| Chain CDR2 | TCGGCATCCTACCTGCAGAGC | 105 |
| MCSP | | |
| Kabat Light | CAGCAATATAACAGCTATCCTCTGACG | 107 |
| Chain CDR3 | | |
| MCSP | | |

It is known that several mechanism are involved in the therapeutic efficacy of anti-MCSP antibodies, including binding to MCSP, blocking of MCSP ligands, antibody dependent cellular cytotoxicity (ADCC), inhibition of melanoma cell adhesion and migration, inhibition of chemotactic responses to fibronectin, and inhibition/killing of pericytes, inhibition of cell spreading on ECM proteins such as collagen and fibronectin, inhibition of cytoskeletal reorganization, and inhibition of MCSP-mediated signal transduction networks (e.g., FAK and ERK networks). Thus, the ABMs of the invention can be used for any of these purposes.

The murine monoclonal antibody 225.28S has been used in the radioimmunodetection of malignant melanoma. Buraggi et al., Nuklearmedizin 25(6):220-224 (1986). More recently, it has been cloned in single-chain Fv configuration for soluble expression in bacteria. Neri et al., J. Invest. Dermatol. 107(2):164-170 (1996), which is incorporated herein by reference in its entirety.

Chimeric mouse/human antibodies have been described. See, for example, Morrison, S. L. et al., PNAS 11:6851-6854 (November 1984); European Patent Publication No. 173494; Boulianna, G. L, at al., Nature 312:642 (December 1984); Neubeiger, M. S. et al., Nature 314:268 (March 1985); European Patent Publication No.125023; Tan et al., J. Immunol. 135:8564 (November 1985); Sun, L. K et al., Hybridoma 5(1):517 (1986); Sahagan et al., J. Immunol. 137:1066-1074 (1986). See generally, Muron, Nature 312:597 (December 1984); Dickson, Genetic Engineering News 5(3) (March 1985); Marx, Science 229:455 (August 1985); and Morrison, Science 229:1202-1207 (September 1985).

In a particularly preferred embodiment, the chimeric ABM of the present invention is a humanized antibody. Methods for humanizing non-human antibodies are known in the art. For example, humanized ABMs of the present invention can be prepared according to the methods of U.S. Pat. No. 5,225,539 to Winter; U.S. Pat. No. 6,180,370 to Queen et al.; U.S. Pat. No. 6,632,927 to Adair et al.; U.S. Pat. Appl. Pub. No. 2003/0039649 to Foote; U.S. Pat. Appl. Pub. No.2004/0044187 to Sato et al.; or U.S. Pat. Appl. Pub. No.2005/0033028 to Leung et al., the entire contents of each of which are hereby incorporated by reference. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. The subject humanized anti-MCSP antibodies will generally comprise constant regions of human immunoglobulins, such as IgG1.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method of selecting the human framework sequence is to compare the sequence of each individual subregion of the full rodent framework (i.e., FR1, FR2, FR3, and FR4) or some combination of the individual subregions (e.g., FR1 and FR2) against a library of known human variable region sequences that correspond to that framework subregion (e.g., as determined by Kabat numbering), and choose the human sequence for each subregion or combination that is the closest to that of the rodent (Leung U.S. Patent Application Publication No. 2003/0040606A1, published Feb. 27, 2003) (the entire contents of which are hereby incorporated by reference). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)) (the entire contents of each of which are hereby incorporated by reference).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models can be generated using computer programs familiar to those skilled in the art (e.g. Insightll, accelrys inc (former MSI), or at http://swissmodel.expasv.org/ described by Schwede et al., Nucleic Acids Res. 2003 (13):3381-3385). Inspection of these models permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as maintained affinity for the target antigen(s), is achieved. In general, the hypervariable region residues are directly and most substantially involved in influencing antigen binding. In a particular embodiment, the ABM of the invention comprises an antibody light chain variable region with a Proline at position 46 (Kabat). In another embodiment, the ABM of the invention comprises an antibody heavy chain variable region with one or more of a phenylalanine residue at position 27, a serine residue at position 30, or a serine or threonine residue at position 94. These residues may either be naturally occurring in the particular light or heavy chain variable region, or may be introduced by amino acid substitution.

In one embodiment, the antibodies of the present invention comprise a human Fc region. In a specific embodiment, the human constant region is IgG1, as set forth in SEQ ID NOs 109 and 110, and set forth below:

Human IgG1 Constant Region Nucleotide Sequence
(SEQ ID NO:110)
ACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGA
ATCACAAGCCCAGCAACACCAAGGTGGACAAGAAAGCAGAGCCCAAATCT
TGTGACAAAACTCACACATGGCCACCGTGCCCAGCAGCTGAACTCCTGGG
GGGACCGTCAGTCTTCCTCTTCCCCCCAAAACCCAAGGACACCCTCATGA
TCTCCCGGAGCCCTGAGGTCACATGCGTGGTGGTGGACGTGAGCCACGAA
GACCCTGAGGTCAAGTTCAACTGGTACGTGGACGGCGTGGAGGTGCATAA
TGCCAAGACAAAGCCGCGGGAGGAGCAGTACAACAGCACGTACCGTGTGG
TCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCAT
CTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTG
AAAGGCTTCTATCCCAGCGACATCGCCGTGGAGTGGGAGAGCAATGGGCA
GCCGGAGAACAACTACAAGACCACGCCTCCCGTGCTGGACTCCGACGGCT
CCTTCTTCCTCTACAGCAAGCTCACCGTGGACAAGAGCAGGTGGCAGCAG
GGGAACGTCTTCTCATGCTCCGTGATGCATGAGGCTCTGCACAACCACTA
CACGCAGAAGAGCCTCTCCCTGTCTCCGGGTAAATGA
Human IgG1 Constant Region Amino Acid Sequence
(SEQ ID NO:109)
TKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKAEPSC
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

However, variants and isoforms of the human Fc region are also encompassed by the present invention. For example, variant Fc regions suitable for use in the present invention can be produced according to the methods taught in U.S. Pat. No. 6,737,056 to Presta (Fc region variants with altered effector function due to one or more amino acid modifications); or in U.S. Pat. Appl. Nos. 60/439,498; 60/456,041; 60/514,549; or WO 2004/063351 (variant Fc regions with increased binding affinity due to amino acid modification.); or in U.S. Pat. No. 10/672,280 or WO 2004/099249 (Fc variants with altered binding to FcgammaR due to amino acid modification), the contents of each of which are incorporated herein by reference in their entirety.

In another embodiment, the antigen binding molecules of the present invention are engineered to have enhanced binding affinity according to, for example, the methods disclosed in U.S. Pat. Appl. Pub. No. 2004/0132066 to Balint et al., the entire contents of which are hereby incorporated by reference.

In one embodiment, the antigen binding molecule of the present invention is conjugated to an additional moiety, such as a radiolabel or a toxin. Such conjugated ABMs can be produced by numerous methods that are well known in the art.

A variety of radionuclides are applicable to the present invention and those skilled in the art are credited with the ability to readily determine which radionuclide is most appropriate under a variety of circumstances. For example, ¹³¹iodine is a well known radionuclide used for targeted immunotherapy. However, the clinical usefulness of ¹³¹iodine can be limited by several factors including: eight-day physical half-life; dehalogenation of iodinated antibody both in the blood and at tumor sites; and emission characteristics (eg, large gamma component) which can be suboptimal for localized dose deposition in tumor. With the advent of superior chelating agents, the opportunity for attaching metal chelating groups to proteins has increased the opportunities to utilize other radionuclides such as ¹¹¹lindium and ⁹⁰yttrium. ⁹⁰Yttrium provides several benefits for utilization in radioimmunotherapeutic applications: the 64 hour half-life of ⁹⁰yttrium is long enough to allow antibody accumulation by tumor and, unlike eg, ¹³¹iodine, ⁹⁰yttrium is a pure beta emitter of high energy with no accompanying gamma irradiation in its decay, with a range in tissue of 100 to 1000 cell diameters. Furthermore, the minimal amount of penetrating radiation allows for outpatient administration of 90yttrium-labeled antibodies. Additionally, internalization of labeled antibody is not required for cell killing, and the local emission of ionizing radiation should be lethal for adjacent tumor cells lacking the target antigen.

With respect to radiolabeled anti-MCSP antibodies, therapytherewith can also occur using a single therapy treatment or using multiple treatments. Because of the radionuclide component, it is preferred that prior to treatment, peripheral stem cells (“PSC”) or bone marrow (“BM”) be “harvested” for patients experiencing potentially fatal bone marrow toxicity resulting from radiation. BM and/or PSC are harvested using standard techniques, and then purged and frozen for possible reinfusion. Additionally, it is most preferred that prior to treatment a diagnostic dosimetry study using a diagnostic labeled antibody (eg, using l indium) be conducted on the patient, a purpose of which is to ensure that the therapeutically labeled antibody (eg, using ⁹⁰yttrium) will not become unnecessarily “concentrated” in any normal organ or tissue.

In a preferred embodiment, the present invention is directed to an isolated polynucleotide comprising a sequence that encodes a polypeptide having an amino acid sequence in Table 7 below (SEQ ID NOS: 2-52 even). The invention is further directed to an isolated nucleic acid comprising a sequence at least 80%, 85%, 90%, 95%, 96%, 97%, 98% or 99% identical to a nucleotide sequence shown in Table 6 below (SEQ ID NOS:1-51 odd). In another embodiment, the invention is directed to an isolated nucleic acid comprising a sequence that encodes a polypeptide having an amino acid sequence at least 80%, 85%, 90%, 95%, 96%, 97%, 98% or 99% identical to an amino acid sequence in Table 7(SEQ ID NOS: 2-52 even). The invention also encompasses an isolated nucleic acid comprising, a sequence that encodes a polypeptide having the amino acid sequence of any of the constructs in Table 7 (SEQ ID NOS: 2-52 even) with conservative amino acid substitutions.

TABLE 6

SEQ
ID

| | | |
|---|---|---|
| CONSTRUCT | NUCLEOTIDE SEQUENCE | NO |

| | | |
|---|---|---|
| VH 225.28S | CAGGTGAAGCTGCAGCAGTCAGGAGGGGGCT | 1 |
| | TGGTGCAACCTGGAGGATCCATGAAACTCTCC | |
| | TGTGTTGTCTCTGGATTCACTTTCAGTAATTAC | |
| | TGGATGAACTGGGTCCGCCAGTCTCCAGAGAA | |
| | GGGGCTTGAGTGGATTGCAGAAATTAGATTGA | |
| | AATCCAATAATTTTGGAAGATATTATGCGGAG | |
| | TCTGTGAAAGGGAGGTTCACCATCTCAAGAGA | |
| | TGATTCCAAAAGTAGTGCCTACCTGCAAATGA | |
| | TCAACCTAAGAGCTGAAGATACTGGCATTTAT | |
| | TACTGTACCAGTTATGGTAACTACGTTGGGCA | |
| | CTATTTTGACCACTGGGGCCAAGGGACCACGG | |
| | TCACCGTCTCGAGT | |
| M-HHA | GAAGTGCAGCTGGTGGAGTCTGGAGGAGGCT | 3 |
| | TGGTCAAGCCTGGCGGGTCCCTGCGGCTCTCC | |
| | TGTGCAGCCTCCGGATTCACATTTAGCAACTA | |
| | TTGGATGAACTGGGTGCGGCAGGCTCCTGGAA | |
| | AGGGCCTCGAGTGGGTGGGAGAGATCAGATT | |
| | GAAATCCAATAACTTCGGAAGATATTACGCTG | |
| | CAAGCGTGAAGGGCCGGTTCACCATCAGCAG | |
| | AGATGATTCCAAGAACACGCTGTACCTGCAGA | |
| | TGAACAGCCTGAAGACCGAGGATACGGCCGT | |
| | GTATTACTGTACCACATACGGCAACTACGTTG | |
| | GGCACTACTTCGACCACTGGGGCCAAGGGACC | |
| | ACCGTCACCGTCTCCAGT | |
| M-HHB | GAAGTGCAGCTGGTGGAGTCTGGAGGAGGCT | 5 |
| | TGGTCAAGCCTGGCGGGTCCCTGCGGCTCTCC | |
| | TGTGCAGCCTCCGGATTCACATTTAGCAACTA | |
| | TTGGATGAACTGGGTGCGGCAGGCTCCTGGAA | |
| | AGGGCCTCGAGTGGGTGGGAGAGATCAGATT | |
| | GAAATCCAATAACTTCGGAAGATATTACGCTG | |
| | AGAGCGTGAAGGGCCGGTTCACCATCAGCAG | |
| | AGATGATTCCAAGAACACGCTGTACCTGCAGA | |
| | TGAACAGCCTGAAGACCGAGGATACGGCCGT | |
| | GTATTACTGTACCTCCTACGGCAACTACGTTG | |
| | GGCACTACTTCGACCACTGGGGCCAAGGGACC | |
| | ACCGTCACCGTCTCCAGT | |
| M-HHC | GAAGTGCAGCTGGTGGAGTCTGGAGGAGGCT | 7 |
| | TGGTCAAGCCTGGCGGGTCCCTGCGGCTCTCC | |
| | TGTGCAGCCTCCGGATTCACATTTAGCAACTA | |
| | TTGGATGAACTGGGTGCGGCAGGCTCCTGGAA | |
| | AGGGCCTCGAGTGGGTGGCCAACATCAGATTG | |
| | AAATCCAATAACTTCGGAAGATATTACGCTGA | |
| | GAGCGTGAAGGGCCGGTTCACCATCAGCAGA | |
| | GATGATTCCAAGAACACGCTGTACCTGCAGAT | |
| | GAACAGCCTGAAGACCGAGGATACGGCCGTG | |
| | TATTACTGTACCTCCTACGGCAACTACGTTGG | |
| | GCACTACTTCGACCACTGGGGCCAAGGGACCA | |
| | CCGTCACCGTCTCCAGT | |
| M-HLA | CAGGTGCAGCTGGTGCAGTCTGGCGCTGAGGT | 9 |
| | GAAGAAGCCTGGCGCCTCGGTGAAGGTCTCCT | |
| | GCAAGGCCTCCGGATACACATTCACCAACTAT | |
| | TGGATGAACTGGGTGCGACAGGCTCCTGGACA | |
| | AGGGCTCGAGTGGATGGGCGAGATCAGATTG | |
| | AAATCCAATAACTTCGGAAGATATTACGCACA | |
| | GAAGTTTCAGGGCAGAGTCACAATGACACGG | |
| | GACACGTCCACTTCCACCGTCTACATGGAGCT | |
| | GAGCAGCCTGAGATCCGAGGATACGGCCGTCT | |
| | ACTACTGCGCAAGATACGGCAACTACGTTGGG | |
| | CACTACTTCGACCACTGGGGCCAAGGGACCAC | |
| | CGTCACCGTCTCCAGT | |
| M-HLB | CAGGTGCAGCTGGTGCAGTCTGGCGCTGAGGT | 11 |
| | GAAGAAGCCTGGCGCCTCGGTGAAGGTCTCCT | |
| | GCAAGGCCTCCGGATACACATTCACCAACTAT | |
| | TGGATGAACTGGGTGCGACAGGCTCCTGGACA | |
| | AGGGCTCGAGTGGATGGGCGAGATCAGATTG | |
| | AAATCCAATAACTTCGGAAGATATTACGCACA | |
| | GAAGTTTCAGGGCAGAGTCACAATCACACGG | |
| | GACACGAGCATGTCCACCGCCTACATGGAGCT | |
| | GAGCAGCCTGAGATCCGAGGATACGGCCGTCT | |
| | ACTACTGCGCAGCCTACGGCAACTACGTTGGG | |
| | CACTACTTCGACCACTGGGGCCAAGGGACCAC | |
| | CGTCACCGTCTCCAGT | |
| M-HLC | CAGGTGCAGCTGGTGCAGTCTGGCGCTGAGGT | 13 |
| | GAAGAAGCCTGGCGCCTCGGTGAAGGTCTCCT | |
| | GCAAGGCCTCCGGATACACATTCACCAACTAT | |
| | TGGATGAACTGGGTGCGACAGGCTCCTGGACA | |
| | AGGGCTCGAGTGGATGGGCGAGATCAGATTG | |
| | AAATCCAATAACTTCGGAAGATACTACGCAGA | |
| | GTCCGTGAAGGGCAGAGTCACAATCACACGG | |
| | GACACGAGCATGTCCACCGCCTACATGGAGCT | |
| | GAGCAGCCTGAGATCCGAGGATACGGCCGTCT | |
| | ACTACTGCGCAGCCTACGGCAACTACGTTGGG | |
| | CACTACTTCGACCACTGGGGCCAAGGGACCAC | |
| | CGTCACCGTCTCCAGT | |
| M-HLD | CAGGTGCAGCTGGTGCAGTCTGGCGCTGAGGT | 15 |
| | GAAGAAGCCTGGCGCCTCGGTGAAGGTCTCCT | |
| | GCAAGGCCTCCGGATTCACATTCAGCAACTAT | |
| | TGGATGAACTGGGTGCGACAGGCTCCTGGACA | |
| | AGGGCTCGAGTGGATGGGCGAGATCAGATTG | |
| | AAATCCAATAACTTCGGAAGATACTACGCAGA | |
| | GTCCGTGAAGGGCAGAGTCACAATCACACGG | |
| | GACACGAGCATGTCCACCGCCTACATGGAGCT | |
| | GAGCAGCCTGAGATCCGAGGATACGGCCGTCT | |
| | ACTACTGCGCAGCCTACGGCAACTACGTTGGG | |
| | CACTACTTCGACCACTGGGGCCAAGGGACCAC | |
| | CGTCACCGTCTCCAGT | |
| M-HLE1 | GAAGTGCAGCTGGTGGAGTCTGGAGGAGGCT | 17 |
| | TGGTCAAGCCTGGCGGGTCCCTGCGGCTCTCC | |
| | TGTGCAGCCTCCGGATTCACATTTAGCAACTA | |
| | TTGGATGAACTGGGTGCGGCAGGCACCAGGA | |
| | AAGGGACTCGAGTGGGTGGGCGAAATCCGGT | |
| | TGAAATCCAATAACTTCGGAAGATACTACGCA | |
| | CAGAAGTTCCAGGAGAGAGTCACAATCACAC | |
| | GGGACATGAGCACCTCCACCGCCTACATGGAG | |
| | CTGAGCAGCCTGAGATCCGAGGATACGGCCGT | |
| | CTACTACTGCGCAGCCTACGGCAACTACGTTG | |
| | GGCACTACTTCGACCACTGGGGCCAAGGGACC | |
| | ACCGTCACCGTCTCCAGT | |
| M-HLE2 | GAAGTGCAGCTGGTGGAGTCTGGAGGAGGCT | 19 |
| | TGGTCAAGCCTGGCGGGTCCCTGCGGCTCTCC | |
| | TGTGCAGCCTCCGGATTCACATTTAGCAACTA | |
| | TTGGATGAACTGGGTGCGGCAGGCACCAGGA | |
| | AAGGGACTCGAGTGGGTGGGCGAAATCCGGT | |
| | TGAAATCCAATAACTTCGGAAGATACTACGCA | |
| | GAGTCCGTGAAGGGAAGAGTCACAATCACAC | |
| | GGGACATGAGCACCTCCACCGCCTACATGGAG | |
| | CTGAGCAGCCTGAGATCCGAGGATACGGCCGT | |
| | CTACTACTGCGCAGCCTACGGCAACTACGTTG | |
| | GGCACTACTTCGACCACTGGGGCCAAGGGACC | |
| | ACCGTCACCGTCTCCAGT | |
| M-HLF | GAAGTGCAGCTGGTGGAGTCTGGAGGAGGCT | 21 |
| | TGGTCCAGCCTGGCGGGTCCCTGCGGCTCTCC | |
| | TGTGCAGCCTCCGGATTCACATTTAGCAGCTA | |
| | TTGGATGAGCTGGGTGCGGCAGGCTCCTGGAA | |
| | AGGGCCTCGAGTGGGTGGCCGAGATCAGATT | |
| | GAAATCCAATAACTTCGGAAGATATTACGCTG | |
| | CAAGCGTGAAGGGCCGGTTCACCATCAGCAG | |
| | AGATGATTCCAAGAACACGCTGTACCTGCAGA | |
| | TGAACAGCCTGAAGACCGAGGATACGGCCGT | |
| | GTATTACTGTACCACATACGGCAACTACGTTG | |
| | GGCACTACTTCGACCACTGGGGCCAAGGGACC | |
| | ACCGTCACCGTCTCCAGT | |
| M-HLG | GAAGTGCAGCTGGTGGAGTCTGGAGGAGGCT | 23 |
| | TGGTCCAGCCTGGCGGGTCCCTGCGGCTCTCC | |
| | TGTGCAGCCTCCGGATTCACATTTAGCAACTA | |
| | TTGGATGAACTGGGTGCGGCAGGCTCCTGGAA | |
| | AGGGCCTCGAGTGGGTGGCCGAGATCAGATT | |
| | GAAATCCAATAACTTCGGAAGATATTACGCTG | |
| | CAAGCGTGAAGGGCCGGTTCACCATCAGCAG | |
| | AGATGATTCCAAGAACACGCTGTACCTGCAGA | |
| | TGAACAGCCTGAAGACCGAGGATACGGCCGT | |
| | GTATTACTGTACCACATACGGCAACTACGTTG | |
| | GGCACTACTTCGACCACTGGGGCCAAGGGACC | |
| | ACCGTCACCGTCTCCAGT | |
| VH Signal | ATGGACTGGACCTGGAGGATCCTCTTCTTGGT | 25 |
| Sequence | GGCAGCAGCCACAGGAGCCCACTCC | |
| VL 225.28S | GATATCGAGCTCACCCAATCTCCAAAATTCAT | 27 |
| | GTCCACATCAGTAGGAGACAGGGTCAGCGTC | |
| | ACCTGCAAGGCCAGTCAGAATGTGGATACTAA | |
| | TGTAGCGTGGTATCAACAAAAACCAGGGCAA | |
| | TCTCCTGAACCACTGCTTTTCTCGGCATCCTAC | |
| | CGTTACACTGGAGTCCCTGATCGCTTCACAGG | |
| | CAGTGGATCTGGGACAGATTTCACTCTCACCA | |
| | TCAGCAATGTGCAGTCTGAAGACTTGGCAGAG | |
| | TATTTCTGTCAGCAATATAACAGCTATCCTCTG | |
| | ACGTTCGGTGGCGGCACCAAGCTGGAAATCA | |
| | AA | |
| M-KV1 | GATATCCAGTTGACCCAGTCTCCATCCTTCCTG | 29 |
| | TCTGCATCTGTGGGCGACCGGGTCACCATCAC | |
| | CTGCAGGGCCAGTCAGAATGTGGATACTAACT | |
| | TAGCTTGGTACCAGCAGAAGCCAGGGAAAGC | |
| | ACCTAAGCTCCTGATCTATTCGGCATCCTACC | |
| | GTTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACGGTG | |
| M-KV2 | GATATCCAGTTGACCCAGTCTCCATCCTTCCTG | 31 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGAAAGC | |
| | ACCTGAGCTCCTGATCTATTCGGCATCCTACC | |
| | GTTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACGGTG | |
| M-KV3 | GATATCCAGTTGACCCAGTCTCCATCCTTCCTG | 33 |
| | TCTGCATCTGTGGGCGACCGGGTCACCGTCAC | |
| | CTGCAAGGCCAGTCAGAATGTGGATACTAACG | |
| | TGGCTTGGTACCAGCAGAAGCCAGGGAAAGC | |
| | ACCTGAGCCTCTTCTGTTCTCGGCATCCTACCG | |
| | TTACACTGGCGTCCCATCAAGGTTCAGCGGCA | |
| | GTGGATCCGGGACAGAGTTCACTCTCACAATC | |
| | TCAAGCCTGCAACCTGAAGATTTCGCAACTTA | |
| | CTACTGTCAACAGTACAATAGTTACCCTCTGA | |
| | CGTTCGGCGGAGGTACCAAGGTGGAGATCAA | |
| | GCGTACGGTG | |
| M-KV4 | GATATCCAGTTGACCCAGTCTCCATCCTTCCTG | 35 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGAAAGC | |
| | ACCTGAGCCTCTTCTGTTCTCGGCATCCTACCG | |
| | TTACACTGGCGTCCCATCAAGGTTCAGCGGCA | |
| | GTGGATCCGGGACAGAGTTCACTCTCACAATC | |
| | TCAAGCCTGCAACCTGAAGATTTCGCAACTTA | |
| | CTACTGTCAACAGTACAATAGTTACCCTCTGA | |
| | CGTTCGGCGGAGGTACCAAGGTGGAGATCAA | |
| | GCGTACGGTG | |
| M-KV5 | GATATCCAGTTGACCCAGTCTCCATCCTTCCTG | 37 |
| | TCTGCATCTGTGGGCGACCGGGTCACCATCAC | |
| | CTGCAAGGCCAGTCAGAATGTGGATACTAACG | |
| | TGGCTTGGTACCTGCAGAAGCCCGGGCAGTCT | |
| | CCTCAGCTCCTGATCTATTCGGCATCCTACCGT | |
| | TACACTGGCGTCCCATCAAGGTTCAGCGGCAG | |
| | TGGATCCGGGACAGAGTTCACTCTCACAATCT | |
| | CAAGCCTGCAACCTGAAGATTTCGCAACTTAC | |
| | TACTGTCAACAGTACAATAGTTACCCTCTGAC | |
| | GTTCGGCGGAGGTACCAAGGTGGAGATCAAG | |
| | CGTACGGTG | |
| M-KV6 | GATATCCAGTTGACCCAGTCTCCATCCTTCCTG | 39 |
| | TCTGCATCTGTGGGCGACCGGGTCACCATCAC | |
| | CTGCAAGGCCAGTCAGAATGTGGATACTAACG | |
| | TGGCTTGGTTCCAGCAGAGGCCCGGGCAGTCT | |
| | CCTCGACGACTGATCTATTCGGCATCCTACCG | |
| | TTACACTGGCGTCCCATCAAGGTTCAGCGGCA | |
| | GTGGATCCGGGACAGAGTTCACTCTCACAATC | |
| | TCAAGCCTGCAACCTGAAGATTTCGCAACTTA | |
| | CTACTGTCAACAGTACAATAGTTACCCTCTGA | |
| | CGTTCGGCGGAGGTACCAAGGTGGAGATCAA | |
| | GCGTACGGTG | |
| M-KV7 | GATATCCAGTTGACCCAGTCTCCATCCTTCCTG | 41 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGAAAGC | |
| | ACCTAAGCCTCTGATCTATTCGGCATCCTACC | |
| | GGTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACGGTG | |
| M-KV8 | GATATCCAGTTGACCCAGTCTCCATCCTTCCTG | 43 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGAAAGC | |
| | ACCTAAGCTTCTGATCTTCTCGGCATCCTACCG | |
| | TTACACTGGCGTCCCATCAAGGTTCAGCGGCA | |
| | GTGGATCCGGGACAGAGTTCACTCTCACAATC | |
| | TCAAGCCTGCAACCTGAAGATTTCGCAACTTA | |
| | CTACTGTCAACAGTACAATAGTTACCCTCTGA | |
| | CGTTCGGCGGAGGTACCAAGGTGGAGATCAA | |
| | GCGTACGGTG | |
| M-KV9 | GATATCCAGTTGACCCAGTCTCCATCCTTCCTG | 45 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGCAGGC | |
| | ACCTAGGCCTCTGATCTATTCGGCATCCTACC | |
| | GGTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACGGTG | |
| M-KV10 | GATATCCAGTTGACCCAGTCTCCATCCTTCCTG | 47 |
| | TCTGCATCTGTGGGCGACCGGGTCACCATCAC | |
| | CTGCAGGGCCAGTCAGAATGTGGATACTAACT | |
| | TAGCTTGGTACCAGCAGAAGCCAGGGCAGGC | |
| | ACCTAGGCCTCTGATCTATTCGGCATCCTACC | |
| | GGTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACG | |
| M-KV11 | GATATCCAGTTGACCCAGTCTCCATCCTTCCTG | 49 |
| | TCTGCATCTGTGGGCGACCGGGTCACCATCAC | |
| | CTGCAGGGCCAGTCAGAATGTGGATACTAACG | |
| | TGGCTTGGTATCAGCAGAAGCCAGGGCAGGC | |
| | ACCTAGGCCTCTGATCTATTCGGCATCCTACC | |
| | GGTACACTGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACG | |
| M-KV12 | GATATCCAGTTGACCCAGTCTCCATCCTTCCTG | 51 |
| | TCTGCATCTGTGGGCGACCGGGTCACCATCAC | |
| | CTGCAAGGCCAGTCAGAATGTGGATACTAACG | |
| | TGGCTTGGTACCAGCAGAAGCCAGGGCAGGC | |
| | ACCTAGGCCTCTGATCTATTCGGCATCCTACCT | |
| | GCAGAGCGGCGTCCCATCAAGGTTCAGCGGC | |
| | AGTGGATCCGGGACAGAGTTCACTCTCACAAT | |
| | CTCAAGCCTGCAACCTGAAGATTTCGCAACTT | |
| | ACTACTGTCAACAGTACAATAGTTACCCTCTG | |
| | ACGTTCGGCGGAGGTACCAAGGTGGAGATCA | |
| | AGCGTACG | |
| VL Signal | ATGAGGGTCCCCGCTCAGCTCCTGGGCCTCCT | 53 |
| Sequence | GCTGCTCTGGTTCCCAGGTGCCAGGTGT | |
| Constant- | GTGGCTGCACCATCTGTCTTCATCTTCCCGCCA | 55 |
| Light | TCTGATGAGCAGTTGAAATCTGGAACTGCCTC | |
| | TGTTGTGTGCCTGCTGAATAACTTCTATCCCAG | |
| | AGAGGCCAAAGTACAGTGGAAGGTGGATAAC | |
| | GCCCTCCAATCGGGTAACTCCCAGGAGAGTGT | |
| | CACAGAGCAGGACAGCAAGGACAGCACCTAC | |
| | AGCCTCAGCAGCACCCTGACGCTGAGCAAAG | |
| | CAGACTACGAGAAACACAAAGTCTACGCCTG | |
| | CGAAGTCACCCATCAGGGCCTGAGCTCGCCCG | |
| | TCACAAAGAGCTTCAACAGGGGAGAGTGTTA | |
| | G | |

TABLE 7

SEQ
ID

| | | |
|---|---|---|
| CONSTRUCT | AMINO ACID SEQUENCE | NO |

| | | |
|---|---|---|
| 225.28S VH | QVKLQQSGGGLVQPGGSMKLSCVVSGFTFSNYWMNW | 2 |
| | VRQSPEKGLEWIAEIRLKSNNFGRYYAESVKGRFTI | |
| | SRDDSKSSAYLQMINLRAEDTGIYYCTSYGNYVGHY | |
| | FDHWGQGTTVTVSS | |
| M-HHA | EVQLVESGGGLVKPGGSLRLSCAASGFTFSNYWMN | 4 |
| | WVRQAPGKGLEWVGEIRLKSNNFGRYYAASVKGRFT | |
| | ISRDDSKNTLYLQMNSLKTEDTAVYYCTTYGNYVGH | |
| | YFDHWGQGTTVTVSS | |
| M-HHB | EVQLVESGGGLVKPGGSLRLSCAASGFTFSNYWMN | 6 |
| | WVRQAPGKGLEWVGEIRLKSNNEGRYYAESVKGRF | |
| | TISRDDSKNTLYLQMNSLKTEDTAVYYCTSYGNYV | |
| | GHYFDHWGQGTTVTVSS | |
| M-HHC | EVQLVESGGGLVKPGGSLRLSCAASGFTFSNYWMN | 8 |
| | WVRQAPGKGLEWVANIRLKSNNFGRYYAESVKGRF | |
| | TISRDDSKNTLYLQMNSLKTEDTAVYYCTSYGNYV | |
| | GHYFDHWGQGTTVTVSS | |
| M-HLA | QVQLVQSGAEVKKPGASVKVSCKASGYTFTNYWMN | 10 |
| | WVRQAPGQGLEWMGEIRLKSNNFGRYYAQKFQGRV | |
| | TMTRDTSTSTVYMELSSLRSEDTAVYYCARYGNYV | |
| | GHYFDHWGQGTTVTVSS | |
| M-HLB | QVQLVQSGAEVKKPGASVKVSCKASGYTFTNYWMN | 12 |
| | WVRQAPGQGLEWMGEIRLKSNNFGRYYAQKFQGRV | |
| | TITRDTSMSTAYMELSSLRSEDTAVYYCAAYGNYV | |
| | GHYFDHWGQGTTVTVSS | |
| M-HLC | QVQLVQSGAEVKKPGASVKVSCKASGYTFTNYWMN | 14 |
| | WVRQAPGQGLEWMGEIRLKSNNFGRYYAESVKGRVT | |
| | ITRDTSMSTAYMELSSLRSEDTAVYYCAAYGNYVG | |
| | HYFDHWGQGTTVTVSS | |
| M-HLD | QVQLVQSGAEVKKPGASVKVSCKASGFTFSNYWMN | 16 |
| | WVRQAPGQGLEWMGEIRLKSNNFGRYYAESVKGRV | |
| | TITRDTSMSTAYMELSSLRSEDTAVYYCAAYGNYV | |
| | GHYFDHWGQGTTVTVSS | |
| M-HLE1 | EVQLVESGGGLVKPGGSLRLSCAASGFTFSNYWMN | 18 |
| | WVRQAPGKGLEWVGEIRLKSNNFGRYYAQKFQERV | |
| | TITRDMSTSTAYMELSSLRSEDTAVYYCAAYGNYV | |
| | GHYFDHWGQGTTVTVSS | |
| M-HLE2 | EVQLVESGGGLVKPGGSLRLSCAASGFTFSNYWMN | 20 |
| | WVRQAPGKGLEWVGEIRLKSNNFGRYYAESVKGRV | |
| | TITRDMSTSTAYMELSSLRSEDTAVYYCAAYGNYV | |
| | GHYFDHWGQGTTVTVSS | |
| M-HLF | EVQLVESGGGLVQPGGSLRLSCAASGFTFSSYWMS | 22 |
| | WVRQAPGKGLEWVAEIRLKSNNFGRYYAASVKGRF | |
| | TISRDDSKNTLYLQMNSLKTEDTAVYYCTTYGNYV | |
| | GHYFDHWGQGTTVTVSS | |
| M-HLG | EVQLVESGGGLVQPGGSLRLSCAASGFTFSNYWMN | 24 |
| | WVRQAPGKGLEWVAEIRLKSNNFGRYYAASVKGRF | |
| | TISRDDSKNTLYLQNSLKTEDTAVYYCTTYGNYVG | |
| | HYFDHWGQGTTVTVSS | |
| VH Signal | MDWTWRILFLVAAATGAHS | 26 |
| Sequence | | |
| 225.28S VL | DIELTQSPKFMSTSVGDRVSVTCKASQNVDTNVAWY | 28 |
| | QQKPGQSPEPLLFSASYRYTGVPDRFTGSGSGTDFT | |
| | LTISNVQSEDLAEYFCQQYNSYPLTFGGGTKLEIK | |
| M-KV1 | DIQLTQSPSFLSASVGDRVTITCRASQNVDTNLAWY | 30 |
| | QQKPGKAPKLLIYSASYRYTGVPSRFSGSGSGTEFT | |
| | LTISSLQPEDFATYYCQQYNSYPLTFGGGTKVEIKR | |
| | T | |
| M-KV2 | DIQLTQSPSFLSASVGDRVTITCKASQNVDTNVAWY | 32 |
| | QQKPGKAPELLIYSASYRYTGVPSRFSGSGSGTEFT | |
| | LTISSLQPEDFATYYCQQYNSYPLTFGGGTKVEIKR | |
| | T | |
| M-KV3 | DIQLTQSPSFLSASVGDRVTVTCKASQNVDTNVAWY | 34 |
| | QQKPGKAPEPLLFSASYRYTGVPSRFSGSGSGTEFT | |
| | LTISSLQPEDFATYYCQQYNSYPLTFGGGTKVEIKR | |
| | T | |
| M-KV4 | DIQLTQSPSFLSASVGDRVTITCKASQNVDTNVAWY | 36 |
| | QQKPGKAPEPLLFSASYRYTGVPSRFSGSGSGTEFT | |
| | LTISSLQPEDFATYYCQQYNSYPLTFGGGTKVEIKR | |
| | T | |
| M-KV5 | DIQLTQSPSFLSASVGDRVTITCKASQNVDTNVAWY | 38 |
| | LQKPGQSPQLLIYSASYRYTGVPSRFSGSGSGTEFT | |
| | LTISSLQPEDFATYYCQQYNSYPLTFGGGTKVEIKR | |
| | T | |
| M-KV6 | DIQLTQSPSFLSASVGDRVTITCKASQNVDTNVAWF | 40 |
| | QQRPGQSPRRLIYSASYRYTGVPSRFSGSGSGTEFT | |
| | LTISSLQPEDFATYYCQQYNSYPLTFGGGTKVEIKR | |
| | T | |
| M-KV7 | DIQLTQSPSFLSASVGDRVTITCKASQNVDTNVAWY | 42 |
| | QQKPGKAPKPLIYSASYRYTGVPSRFSGSGSGTEFT | |
| | LTISSLQPEDFATYYCQQYNSYPLTFGGGTKVEIKR | |
| | T | |
| M-KV8 | DIQLTQSPSFLSASVGDRVTITCKASQNVDTNVAWY | 44 |
| | QQKPGKAPKLLIFSASYRYTGVPSRFSGSGSGTEFT | |
| | LTISSLQPEDFATYYCQQYNSYPLTFGGGTKVEIKR | |
| | T | |
| M-KV9 | DIQLTQSPSFLSASVGDRVTITCKASQNVDTNVAWY | 46 |
| | QQKPGQAPRPLIYSASYRYTGVPSRFSGSGSGTEFT | |
| | LTISSLQPEDFATYYCQQYNSYPLTFGGGTKVEIKR | |
| | T | |
| M-KV10 | DIQLTQSPSFLSASVGDRVTITCRASQNVDTNLAWY | 48 |
| | QQKPGQAPRPLIYSASYRYTGVPSRFSGSGSGTEFT | |
| | LTISSLQPEDFATYYCQQYNSYPLTFGGGTKVEIKR | |
| | T | |
| M-KV11 | DIQLTQSPSFLSASVGDRVTITCRASQNVDTNVAWY | 50 |
| | QQKPGQAPRPLIYSASYRYTGVPSRFSGSGSGTEFT | |
| | LTISSLQPEDFATYYCQQYNSYPLTFGGGTKVEIKR | |
| | T | |
| M-KV12 | DIQLTQSPSFLSASVGDRVTITCKASQNVDTNVAWY | 52 |
| | QQKPGQAPRPLIYSASYLQSGVPSRFSGSGSGTEFT | |
| | LTISSLQPEDFATYYCQQYNSYPLTFGGGTKVEIKR | |
| | T | |
| VL Signal | MRVPAQLLGLLLLWFPGARC | 54 |
| Sequence | | |

In another embodiment, the present invention is directed to an expression vector and/or a host cell which comprise one or more isolated polynucleotides of the present invention.

Generally, any type of cultured cell line can be used to express the ABM of the present invention. In a preferred embodiment, HEK293-EBNA cells, CHO cells, BHK cells, NSO cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the invention.

The therapeutic efficacy of the ABMs of the present invention can be enhanced by producing them in a host cell that further expresses one or more of the following: a polynucleotide encoding a polypeptide having GnTIII activity, a polynucleotide encoding a polypeptide having ManII activity, or a polynucleotide encoding a polypeptide having GalT activity. In a preferred embodiment, the host cell expresses a polynucleotide encoding a polypeptide having GnTIII activity or ManII activity. In another preferred embodiment, the host cell expresses a polynucleotide encoding a polypetide having GnTIII activity as well as a polynucleotide encoding a polypeptide having ManII activity. In yet another preferred embodiment, the polypeptide having GnTIII activity is a fusion polypeptide comprising the Golgi localization domain of a Golgi resident polypeptide. In another preferred embodiment, the expression of the ABMs of the present invention in a host cell that expresses a polynucleotide encoding a polypeptide having GnTIII activity results in ABMs with increased Fc receptor binding affinity and increased effector function. Accordingly, in one embodiment, the present invention is directed to a host cell comprising (a) an isolated nucleic acid comprising a sequence encoding a polypeptide having GnTIII activity; and (b) an isolated polynucleotide encoding an ABM of the present invention, such as a chimeric, primatized or humanized antibody that binds human MCSP. In a preferred embodiment, the polypeptide having GnTIII activity is a fusion polypeptide comprising the catalytic domain of GnTIII and the Golgi localization domain is the localization domain of mannosidase II. Methods for generating such fusion polypeptides and using them to produce antibodies with increased effector functions are disclosed in U.S. Provisional Pat. Appl. No. 60/495,142 and U.S. Pat. Appl. Publ. No.2004/0241817 Al, the entire contents of each of which are expressly incorporated herein by reference. In another preferred embodiment, the chimeric ABM is a chimeric antibody or a fragment thereof, having the binding specificity of the murine 225.28S monoclonal antibody. In a particularly preferred embodiment, the chimeric antibody comprises a human Fc. In another preferred embodiment, the antibody is primatized or humanized.

In an alternative embodiment, the ABMs of the present invention can be enhanced by producing them in a host cell that has been engineered to have reduced, inhibited, or eliminated activity of at least one fucosyltransferase.

In one embodiment, one or several polynucleotides encoding an ABM of the present invention may be expressed under the control of a constitutive promoter or, alternately, a regulated expression system. Suitable regulated expression systems include, but are not limited to, a tetracycline-regulated expression system, an ecdysone-inducible expression system, a lac-switch expression system, a glucocorticoid-inducible expression system, a temperature-inducible promoter system, and a metallothionein metal-inducible expression system. If several different nucleic acids encoding an ABM of the present invention are comprised within the host cell system, some of them may be expressed under the control of a constitutive promoter, while others are expressed under the control of a regulated promoter. The maximal expression level is considered to be the highest possible level of stable polypeptide expression that does not have a significant adverse effect on cell growth rate, and will be determined using routine experimentation. Expression levels are determined by methods generally known in the art, including Western blot analysis using an antibody specific for the ABM or an antibody specific for a peptide tag fused to the ABM; and Northern blot analysis. In a further alternative, the polynucleotide may be operatively linked to a reporter gene; the expression levels of a chimeric ABM having substantially the same binding specificity of the murine 225.28S monoclonal antibody are determined by measuring a signal correlated with the expression level of the reporter gene. The reporter gene may be transcribed together with the nucleic acid(s) encoding said fusion polypeptide as a single mRNA molecule; their respective coding sequences may be linked either by an internal ribosome entry site (IRES) or by a cap-independent translation enhancer (CITE). The reporter gene may be translated together with at least one nucleic acid encoding a chimeric ABM having substantially the same binding specificity of the murine 225.28S monoclonal antibody such that a single polypeptide chain is formed. The nucleic acids encoding the ABMs of the present invention may be operatively linked to the reporter gene under the control of a single promoter, such that the nucleic acid encoding the fusion polypeptide and the reporter gene are transcribed into an RNA molecule which is alternatively spliced into two separate messenger RNA (mRNA) molecules; one of the resulting mRNAs is translated into said reporter protein, and the other is translated into said fusion polypeptide.

Methods which are well known to those skilled in the art can be used to construct expression vectors containing the coding sequence of an ABM having substantially the same binding specificity of the murine 225.28S monoclonal antibody along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING A LABORATORY MANUAL, Cold Spring Harbor Laboratory, N.Y. (1989) and Ausubel et al., CURRENT PROTOCOLS IN MOLECULAR BIOLOGY, Greene Publishing Associates and Wiley Interscience, N.Y (1989).

A variety of host-expression vector systems maybe utilized to express the coding sequence of the ABMs of the present invention. Preferably, mammalian cells are used as host cell systems transfected with recombinant plasmid DNA or cosmid DNA expression vectors containing the coding sequence of the protein of interest and the coding sequence of the fusion polypeptide. Most preferably, CHO cells, BHK cells, NS0 cells, SP2/0 cells, YO myeloma cells, P3X63 mouse myeloma cells, PER cells, PER.C6 cells or hybridoma cells, other mammalian cells, yeast cells, insect cells, or plant cells are used as host cell system. Some examples of expression systems and selection methods are described in the following references, and references therein: Borth et al., Biotechnol. Bioen. 71(4):266-73 (2000-2001), in Werner et al., Arzneimittelforschung/Drug Res. 48(8):870-80 (1998), in Andersen and Krummen, Curr. Op. Biotechnol. 13:117-123 (2002), in Chadd and Chamow, Curr. Op. Biotechnol. 12:188-194 (2001), and in Giddings, Curr. Op. Biotechnol. 12: 450-454 (2001). In alternate embodiments, other eukaryotic host cell systems may be used, including yeast cells transformed with recombinant yeast expression vectors containing the coding sequence of an ABM of the present invention, such as the expression systems taught in U.S. Pat. Appl. No. 60/344,169 and WO 03/056914 (methods for producing human-like glycoprotein in a non-human eukaryotic host cell) (the contents of each of which are incorporated by reference in their entirety); insect cell systems infected with recombinant virus expression vectors (e.g., baculovirus) containing the coding sequence of a chimeric ABM having substantially the same binding specificity of the murine 225.28S monoclonal antibody; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of the ABM of the invention, including, but not limited to, the expression systems taught in U.S. Pat. No. 6,815,184 (methods for expression and secretion of biologically active polypeptides from genetically engineered duckweed); WO 2004/057002 (production of glycosylated proteins in bryophyte plant cells by introduction of a glycosyl transferase gene) and WO 2004/024927 (methods of generating extracellular heterologous non-plant protein in moss protoplast); and U.S. Pat. Appl. Nos. 60/365,769, 60/368,047, and WO 2003/078614 (glycoprotein processing in transgenic plants comprising a functional mammalian GnTIII enzyme) (the contents of each of which are hereby incorporated by reference in their entirety); or animal cell systems infected with recombinant virus expression vectors (e.g., adenovirus, vaccinia virus) including cell lines engineered to contain multiple copies of the DNA encoding a chimeric ABM having substantially the same binding specificity of the murine 225.28S monoclonal antibody either stably amplified (CHO/dhfr) or unstably amplified in double-minute chromosomes (e.g., murine cell lines). In one embodiment, the vector comprising the polynucleotide(s) encoding the ABM of the invention is polycistronic. Also, in one embodiment the ABM discussed above is an antibody or a fragment thereof. In a preferred embodiment, the ABM is a humanized antibody.

For the methods of this invention, stable expression is generally preferred to transient expression because it typically achieves more reproducible results and also is more amenable to large-scale production, although transient expression is also encompassed by the invention. Rather than using expression vectors which contain viral origins of replication, host cells can be transformed with the respective coding nucleic acids controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows selection of cells which have stably integrated the plasmid into their chromosomes and grow to form foci which in turn can be cloned and expanded into cell lines.

A number of selection systems may be used, including, but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:2026 (1962)), and adenine phosphoribosyltransferase (Lowy et al., Cell 22:817 (1980)) genes, which can be employed in tk-, hgprt- or aprt-cells, respectively. Also, antimetabolite resistance can be used as the basis of selection for dhfr, which confers resistance to methotrexate (Wigler et al., Natl. Acad. Sci. USA 77:3567 (1989); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Colberre-Garapin et al., J. Mol. Biol. 150:1 (1981)); and hygro, which confers resistance to hygromycin (Santerre et al., Gene 30:147 (1984) genes. Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA 85:8047 (1988)); the glutamine synthase system; and ODC (ornithine decarboxylase) which confers resistance to the omithine decarboxylase inhibitor, 2-(difluoromethyl)-DL-ornithine, DFMO (McConlogue, in: Current Communications in Molecular Biology, Cold Spring Harbor Laboratory ed. (1987)).

The present invention is further directed to a method for modifying the glycosylation profile of the ABMs of the present invention that are produced by a host cell, comprising expressing in said host cell a nucleic acid encoding an ABM of the invention and a nucleic acid encoding a polypeptide with GnTIII activity, or a vector comprising such nucleic acids. Preferably, the modified polypeptide is IgG or a fragment thereof comprising the Fc region. In a particularly preferred embodiment the ABM is a humanized antibody or a fragment thereof. Alternatively, or in addition, such host cells may be engineered to have reduced, inhibited, or eliminated activity of at least one fucosyltransferase. In another embodiment, the host cell is engineered to coexpress an ABM of the invention, GnTIII and mannosidase II (ManII).

The modified ABMs produced by the host cells of the invention exhibit increased Fc receptor binding affinity and/or increased effector function as a result of the modification. In a particularly preferred embodiment the ABM is a humanized antibody or a fragment thereof containing the Fc region. Preferably, the increased Fc receptor binding affinity is increased binding to a Fcγ activating receptor, such as the FcγRIIIa receptor. The increased effector function is preferably an increase in one or more of the following: increased antibody-dependent cellular cytotoxicity, increased antibody-dependent cellular phagocytosis (ADCP), increased cytokine secretion, increased immune-complex-mediated antigen uptake by antigen-presenting cells, increased Fc-mediated cellular cytotoxicity, increased binding to NK cells, increased binding to macrophages, increased binding to polymorphonuclear cells (PMNs), increased binding to monocytes, increased crosslinking of target-bound antibodies, increased direct signaling inducing apoptosis, increased dendritic cell maturation, and increased T cell priming.

Effector functions can be measured and/or determined by various assays known to those of skill in the art. Various assays for measuring effector functions, including Fc receptor binding affinity and complement dependent cytotoxicity, are described in US Application Publication No.2004/0241817A1, which is herein incorporat by reference in its entirety. Cytokine secretion can be measured, for example, using a sandwich ELISA, see, e.g., McRae et al., J. Immunol. 164: 23-28 (2000) and the cytokine sandwich ELISA protocol available at www.bdbiosciences.com/pharmingen/protocols, or by the methods described in Takahashi et al., British J. Pharmacol. 137: 315-322 (2002), each of which is herein incorporated by reference in its entirety. Dendritic cell maturation, for example, can be determined using assays as set forth by Kalergis and Ravetch, J. Exp. Med. 195: 1653-59 (2002), which is herein incorporated by reference in its entirety. Examples of phagocytosis and antigen uptake/presentation assays are provided by Gresham et al., J. Exp. Med. 191: 515-28 (2000); Krauss et al., J. Immunol. 153: 1769-77 (1994); and Rafiq et al., J. Clin. Invest. 110: 71-79 (2002), and Hamano et al., J. Immunol. 164: 6113-19 (2000), each of which is herein incorporated by reference in its entirety. Down regulation of cell-surface receptors can be measured, for example, by methods set forth by Liao et al., Blood 83: 2294-2304 (1994), which is herein incorporated by reference in its entirety. General methods, protocols-and assays, can be found in CELL BIOLOGY: A LABORATORY HANDBOOK, Celis, J. E., ed., (2d ed., 1998), which is herein incorporated by reference in its entirety. It is within the skill of one in the art to adapt the above-referenced methods, protocols and assays for use with the present invention.

The present invention is also directed to a method for producing an ABM of the present invention, having modified oligosaccharides in a host cell comprising (a) culturing a host cell engineered to express at least one nucleic acid encoding a polypeptide having GnTIII activity under conditions which permit the production of an ABM according to the present invention, wherein said polypeptide having GnTIII activity is expressed in an amount sufficient to modify the oligosaccharides in the Fc region of said ABM produced by said host cell; and (b) isolating said ABM. In a preferred embodiment, the polypeptide having GnTIII activity is a fusion polypeptide comprising the catalytic domain of GnTIII. In a particularly preferred embodiment, the fusion polypeptide further comprises the Golgi localization domain of a Golgi resident polypeptide.

Preferably, the Golgi localization domain is the localization domain of human mannosidase II or human GnTIII. Alternatively, the Golgi localization domain is selected from the group consisting of: the localization domain of mannosidase I, the localization domain of GnTIII, and the localization domain of a 1-6 core fucosyltransferase. The ABMs produced by the methods of the present invention have increased Fc receptor binding affinity and/or increased effector function. Preferably, the increased effector function is one or more of the following: increased Fc-mediated cellular cytotoxicity (including increased antibody-dependent cellular cytotoxicity), increased antibody-dependent cellular phagocytosis (ADCP), increased cytokine secretion, increased immune-complex-mediated antigen uptake by antigen-presenting cells, increased binding to NK cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. The increased Fc receptor binding affinity is preferably increased binding to Fc activating receptors such as FcγRIIIa. In a particularly preferred embodiment the ABM is a humanized antibody or a fragment thereof.

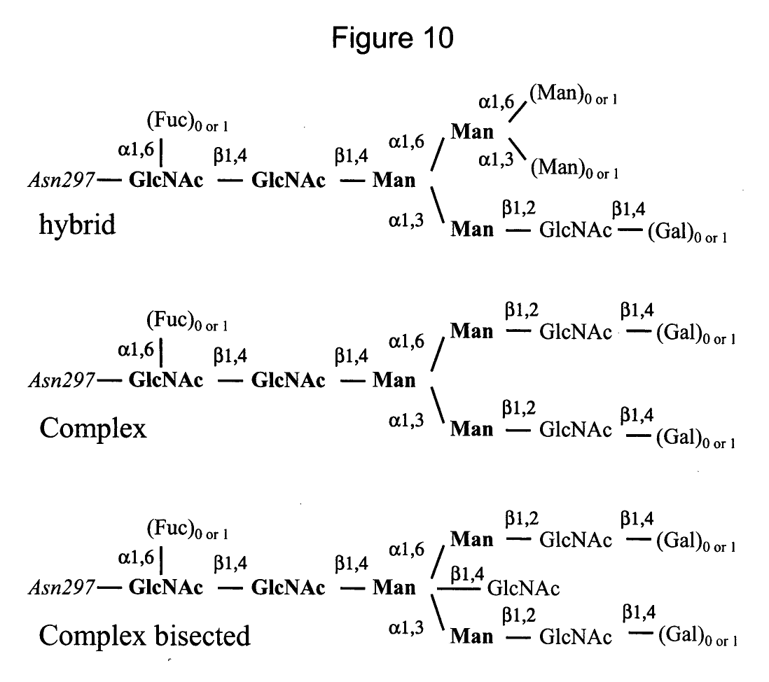

In another embodiment, the present invention is directed to a chimeric ABM having substantially the same binding specificity of the murine 225.28S monoclonal antibody produced by the methods of the invention which has an increased proportion of bisected oligosaccharides in the Fc region of said polypeptide. It is contemplated that such an ABM encompasses antibodies and fragments thereof comprising the Fc region. In a preferred embodiment, the ABM is a humanized antibody. In one embodiment, the percentage of bisected oligosaccharides in the Fc region of the ABM is at least 50%, more preferably, at least 60%, at least 70%, at least 80%, or at least 90%, and most preferably at least 90-95% of the total oligosaccharides. In yet another embodiment, the ABM produced by the methods of the invention has an increased proportion of nonfucosylated oligosaccharides in the Fc region as a result of the modification of its oligosaccharides by the methods of the present invention. In one embodiment, the percentage of nonfucosylated oligosaccharides is at least 50%, preferably, at least 60% to 70%, most preferably at least 75%. The nonfucosylated oligosaccharides may be of the hybrid or complex type. In a particularly preferred embodiment, the ABM produced by the host cells and methods of the invention has an increased proportion of bisected, nonfucosylated oligosaccharides in the Fc region. The bisected, nonfucosylated oligosaccharides may be either hybrid or complex. Specifically, the methods of the present invention may be used to produce ABMs in which at least 15%, more preferably at least 20%, more preferably at least 25%, more preferably at least 30%, more preferably at least 35% of the oligosaccharides in the Fc region of the ABM are bisected, nonfucosylated. The methods of the present invention may also be used to produce polypeptides in which at least 15%, more preferably at least 20%, more preferably at least 25%, more preferably at least 30%, more preferably at least 35% of the oligosaccharides in the Fc region of the polypeptide are bisected hybrid nonfucosylated. (In FIG. 10 the nomenclature of “complex”, “complex bisected”, and “hybrid” oligosaccharides is described.)

In another embodiment, the present invention is directed to a chimeric ABM having substantially the same binding specificity of the murine 225.28S monoclonal antibody and engineered to have increased effector function and/or increased Fc receptor binding affinity, produced by the methods of the invention. Preferably, the increased effector function is one or more of the following: increased Fc-mediated cellular cytotoxicity (including increased antibody-dependent cellular cytotoxicity), increased antibody-dependent cellular phagocytosis (ADCP), increased cytokine secretion, increased immune-complex-mediated antigen uptake by antigen-presenting cells, increased binding to NK cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. In a preferred embodiment, the increased Fc receptor binding affinity is increased binding to a Fc activating receptor, most preferably FcγRIIIa. In one embodiment, the ABM is an antibody, an antibody fragment containing the Fc region, or a fusion protein that includes a region equivalent to the Fc region of an immunoglobulin. In a particularly preferred embodiment, the ABM is a humanized antibody.

The present invention is further directed to pharmaceutical compositions comprising the ABMs of the present invention and a pharmaceutically acceptable carrier.

The present invention is further directed to the use of such pharmaceutical compositions in the method of treatment of cancer. Specifically, the present invention is directed to a method for the treatment of cancer comprising administering a therapeutically effective amount of the pharmaceutical composition of the invention.

In yet another embodiment, the invention relates to an ABM according to the present invention for use as a medicament, in particular for use in the treatment or prophylaxis of cancer or for use in a precancerous condition or lesion. The cancer may be, for example, lung cancer, non small cell lung (NSCL) cancer, bronchioalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

Preferably, said cancer is selected from the group consisting of breast cancer, bladder cancer, head & neck cancer, skin cancer, pancreatic cancer, lung cancer, ovarian cancer, colon cancer, prostate cancer, kidney cancer, and brain cancer.

Yet another embodiment is the use of the ABM according to the present invention for the manufacture of a medicament for the treatment or prophylaxis of cancer. Cancer is as defined above.

Preferably, said cancer is selected from the group consisting of breast cancer, bladder cancer, head & neck cancer, skin cancer, pancreatic cancer, lung cancer, ovarian cancer, colon cancer, prostate cancer, kidney cancer, and brain cancer.

Also preferably, said antigen binding molecule is used in a therapeutically effective amount from about 1.0 mg/kg to about 15 mg/kg.

Also more preferably, said antigen binding molecule is used in a therapeutically effective amount from about 1.5 mg/kg to about 12 mg/kg.

Also more preferably, said antigen binding molecule is used in a therapeutically effective amount from about 1.5 mg/kg to about 4.5 mg/kg.

Also more preferably, said antigen binding molecule is used in a therapeutically effective amount from about 4.5 mg/kg to about 12 mg/kg.

Most preferably, said antigen binding molecule is used in a therapeutically effective amount of about 1.5 mg/kg.

Also most preferably, said antigen binding molecule is used in a therapeutically effective amount of about 4.5 mg/kg.

Also most preferably, said antigen binding molecule is used in a therapeutically effective amount of about 12 mg/kg.

The present invention further provides methods for the generation and use of host cell systems for the production of glycoforms of the ABMs of the present invention, having increased Fc receptor binding affinity, preferably increased binding to Fc activating receptors, and/or having increased effector functions, including antibody-dependent cellular cytotoxicity. The glycoengineering methodology that can be used with the ABMs of the present invention has been described in greater detail in U.S. Pat. No. 6,602,684, U.S. Pat. Appl. Publ. No. 2004/0241817 A1, U.S. Pat. Appl. Publ. No.2003/0175884 A1, Provisional U.S. Patent Application No. 60/441,307 and WO 2004/065540, the entire contents of each of which are incorporated herein by reference in its entirety. The ABMs of the present invention can alternatively be glycoengineered to have reduced fucose residues in the Fc region according to the techniques disclosed in U.S. Pat. Appl. Pub. No. 2003/0157108 (Genentech) or in EP 1 176 195 A1, WO 03/084570, WO 03/085119 and U.S. Pat. Appl. Pub. Nos. 2003/0115614, 2004/093621, 2004/110282, 2004/110704, 2004/132140 (all to Kyowa Hakko Kogyo Ltd.). The contents of each of these documents are hereby incorporated by reference in their entirety. Glycoengineered ABMs of the invention may also be produced in expression systems that produce modified glycoproteins, such as those taught in U.S. Pat. Appl. Pub. No. 60/344,169 and WO 03/056914 (GlycoFi, Inc.) or in WO 2004/057002 and WO 2004/024927 (Greenovation), the contents of each of which are hereby incorporated by reference in their entirety.

Generation of Cell Lines for the Production of Proteins with Altered Glycosylation Pattern

The present invention provides host cell expression systems for the generation of the ABMs of the present invention having modified glycosylation patterns. In particular, the present invention provides host cell systems for the generation of glycoforms of the ABMs of the present invention having an improved therapeutic value. Therefore, the invention provides host cell expression systems selected or engineered to express a polypeptide having GnTIII activity. In one embodiment, the polypeptide having GnTIII activity is a fusion polypeptide comprising the Golgi localization domain of a heterologous Golgi resident polypeptide. Specifically, such host cell expression systems may be engineered to comprise a recombinant nucleic acid molecule encoding a polypeptide having GnTIII, operatively linked to a constitutive or regulated promoter system.

In one specific embodiment, the present invention provides a host cell that has been engineered to express at least one nucleic acid encoding a fusion polypeptide having GnTIII activity and comprising the Golgi localization domain of a heterologous Golgi resident polypeptide. In one aspect, the host cell is engineered with a nucleic acid molecule comprising at least one gene encoding a fusion polypeptide having GnTIII activity and comprising the Golgi localization domain of a heterologous Golgi resident polypeptide.

Generally, any type of cultured cell line, including the cell lines discussed above, can be used as a background to engineer the host cell lines of the present invention. In a preferred embodiment, CHO cells, BHK cells, NSO cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the invention.

The invention is contemplated to encompass any engineered host cells expressing a polypeptide having GnTIII activity, including a fusion polypeptide that comprises the Golgi localization domain of a heterologous Golgi resident polypeptide as defined herein.

One or several nucleic acids encoding a polypeptide having GnTIII activity may be expressed under the control of a constitutive promoter or, alternately, a regulated expression system. Such systems are well known in the art, and include the systems discussed above. If several different nucleic acids encoding fusion polypeptides having GnTIII activity and comprising the Golgi localization domain of a heterologous Golgi resident polypeptide are comprised within the host cell system, some of them may be expressed under the control of a constitutive promoter, while others are expressed under the control of a regulated promoter. Expression levels of the fusion polypeptides having GnTIII activity are determined by methods generally known in the art, including Western blot analysis, Northern blot analysis, reporter gene expression analysis or measurement of GnTIII activity. Alternatively, a lectin may be employed which binds to biosynthetic products of the GnTIII, for example, E₄-PHA lectin. Alternatively, a functional assay which measures the increased Fc receptor binding or increased effector function mediated by antibodies produced by the cells engineered with the nucleic acid encoding a polypeptide with GnTIII activity may be used.

Identification of Transfectants or Transformants that Express the Protein Having a Modified Glycosylation Pattern

The host cells which contain the coding sequence of a chimeric ABM having substantially the same binding specificity of the murine 225.28S monoclonal antibody and which express the biologically active gene products may be identified by at least four general approaches; (a) DNA-DNA or DNA-RNA hybridization; (b) the presence or absence of “marker” gene functions; (c) assessing the level of transcription as measured by the expression of the respective mRNA transcripts in the host cell; and (d) detection of the gene product as measured by immunoassay or by its biological activity.

In the first approach, the presence of the coding sequence of a chimeric ABM having substantially the same binding specificity of the murine 225.28S monoclonal antibody and the coding sequence of the polypeptide having GnTIII activity can be detected by DNA-DNA or DNA-RNA hybridization using probes comprising nucleotide sequences that are homologous to the respective coding sequences, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the coding sequence of the ABM of the invention, or a fragment thereof, and the coding sequence of the polypeptide having GnTIII activity are inserted within a marker gene sequence of the vector, recombinants containing the respective coding sequences can be identified by the absence of the marker gene function. Alternatively, a marker gene can be placed in tandem with the coding sequences under the control of the same or different promoter used to control the expression of the coding sequences. Expression of the marker in response to induction or selection indicates expression of the coding sequence of the ABM of the invention and the coding sequence of the polypeptide having GnTIII activity.

In the third approach, transcriptional activity for the coding region of the ABM of the invention, or a fragment thereof, and the coding sequence of the polypeptide having GnTIII activity can be assessed by hybridization assays. For example, RNA can be isolated and analyzed by Northern blot using a probe homologous to the coding sequences of the ABM of the invention, or a fragment thereof, and the coding sequence of the polypeptide having GnTIII activity or particular portions thereof. Alternatively, total nucleic acids of the host cell may be extracted and assayed for hybridization to such probes.

In the fourth approach, the expression of the protein products can be assessed immunologically, for example by Western blots, immunoassays such as radioimmuno-precipitation, enzyme-linked immunoassays and the like. The ultimate test of the success of the expression system, however, involves the detection of the biologically active gene products.

Generation and Use of ABMs Having Increased Effector Function Including Antibody-Dependent Cellular Cytotoxicity

In preferred embodiments, the present invention provides glycoforms of chimeric ABMs having substantially the same binding specificity of the murine 225.28S monoclonal antibody and having increased effector function including antibody-dependent cellular cytotoxicity. Glycosylation engineering of antibodies has been previously described. See, e.g., U.S. Pat. No. 6,602,684, incorporated herein by reference in its entirety.

Clinical trials of unconjugated monoclonal antibodies (mAbs) for the treatment of some types of cancer have recently yielded encouraging results. Dillman, Cancer Biother. & Radiopharm. 12:223-25 (1997); Deo et al., Immunology Today 18:127 (1997). A chimeric, unconjugated IgG1 has been approved for low-grade or follicular B-cell non-Hodgkin's lymphoma. Dillman, Cancer Biother. & Radiopharm. 12:223-25 (1997), while another unconjugated mAb, a humanized IgG1 targeting solid breast tumors, has also been showing promising results in phase III clinical trials. Deo et al., Immunology Today 18:127 (1997). The antigens of these two mAbs are highly expressed in their respective tumor cells and the antibodies mediate potent tumor destruction by effector cells in vitro and in vivo. In contrast, many other unconjugated mAbs with fine tumor specificities cannot trigger effector functions of sufficient potency to be clinically useful. Frost et al., Cancer 80:317-33 (1997); Surfus et al., J. Immunother. 19:184-91 (1996). For some of these weaker mAbs, adjunct cytokine therapy is currently being tested. Addition of cytokines can stimulate antibody-dependent cellular cytotoxicity (ADCC) by increasing the activity and number of circulating lymphocytes. Frost et al., Cancer 80:317-33 (1997); Surfus et al., J. Immunother. 19:184-91 (1996). ADCC, a lytic attack on antibody-targeted cells, is triggered upon binding of leukocyte receptors to the constant region (Fc) of antibodies. Deo et al., Immunology Today 18:127 (1997).

A different, but complementary, approach to increase ADCC activity of unconjugated IgG1s is to engineer the Fc region of the antibody. Protein engineering studies have shown that FcγRs interact mainly with the hinge region of the IgG molecule. Lund et al., J. Immunol. 157:4963-69 (1996). However, FcγR binding also requires the presence of oligosaccharides covalently attached at the conserved Asn 297 in the CH2 region. Lund et al., J. Immunol. 157:4963-69 (1996); Wright and Morrison, Trends Biotech. 15:26-31 (1997), suggesting that either oligosaccharide and polypeptide both directly contribute to the interaction site or that the oligosaccharide is required to maintain an active CH2 polypeptide conformation. Modification of the oligosaccharide structure can therefore be explored as a means to increase the affinity of the interaction.

An IgG molecule carries two N-linked oligosaccharides in its Fc region, one on each heavy chain. As any glycoprotein, an antibody is produced as a population of glycoforms which share the same polypeptide backbone but have different oligosaccharides attached to the glycosylation sites. The oligosaccharides normally found in the Fc region of serum IgG are of complex bi-antennary type (Wormald et al., Biochemistry 36:130-38 (1997), with a low level of terminal sialic acid and bisecting N-acetylglucosamine (GlcNAc), and a variable degree of terminal galactosylation and core fucosylation. Some studies suggest that the minimal carbohydrate structure required for FcγR binding lies within the oligosaccharide core. Lund et al., J. Immunol. 157:4963-69 (1996)

The mouse- or hamster-derived cell lines used in industry and academia for production of unconjugated therapeutic mAbs normally attach the required oligosaccharide determinants to Fc sites. IgGs expressed in these cell lines lack, however, the bisecting GlcNAc found in low amounts in serum IgGs. Lifely et al., Glycobiology 318:813-22 (1995). In contrast, it was recently observed that a rat myeloma-produced, humanized IgG1 (CAMPATH-1H) carried a bisecting GlcNAc in some of its glycoforms. Lifely et al., Glycobiology 318:813-22 (1995). The rat cell-derived antibody reached a similar maximal in vitro ADCC activity as CAMPATH-1H antibodies produced in standard cell lines, but at significantly lower antibody concentrations.

The CAMPATH antigen is normally present at high levels on lymphoma cells, and this chimeric mAb has high ADCC activity in the absence of a bisecting GlcNAc. Lifely et al., Glycobiology 318:813-22 (1995). In the N-linked glycosylation pathway, a bisecting GlcNAc is added by GnTIII. Schachter, Biochem. Cell Biol. 64:163-81 (1986).

Previous studies used a single antibody-producing CHO cell line, that was previously engineered to express, in an externally-regulated fashion, different levels of a cloned GnT III gene enzyme (Umana, P., et al., Nature Biotechnol. 17:176-180 (1999)). This approach established for the first time a rigorous correlation between expression of GnTIII and the ADCC activity of the modified antibody. Thus, the invention contemplates a recombinant, chimeric or humanized ABM (e.g., antibody) or a fragment thereof with the binding specificity of the murine 225.28S monoclonal antibody, having altered glycosylation resulting from increased GnTIII activity. The increased GnTIII activity results in an increase in the percentage of bisected oligosaccharides, as well as a decrease in the percentage of fucose residues, in the Fc region of the ABM. This antibody, or fragment thereof, has increased Fc receptor binding affinity and increased effector function. In addition, the invention is directed to antibody fragment and fusion proteins comprising a region that is equivalent to the Fc region of immunoglobulins.

Therapeutic Applications of ABMs Produced According to the Methods of the Invention.

In the broadest sense, the ABMs of the present invention can be used to target cells in vivo or in vitro that express MCSP. The cells expressing MCSP can be targeted for diagnostic or therapeutic purposes. In one aspect, the ABMs of the present invention can be used to detect the presence of MCSP in a sample. In another aspect, the ABMs of the present invention can be used to bind MCSP expressing cells in vitro or in vivo for, e.g., identification or targeting. More particularly, the ABMs of the present invention can be used to block or inhibit MCSP binding to an MCSP ligand or, alternatively, target an MCSP expressing cell for destruction. In one embodiment, the MCSP expressing cells are pericytes. Also, the ABMs of the invention can be used to inhibit melanoma cell adhesion and migration, to inhibit of chemotactic responses to fibronectin, and to inhibit pericytes, to inhibit cell spreading on ECM proteins such as collagen and fibronectin, to inhibit FAK and ECR signal transduction networks, and to inhibit or reduce MCSP-mediated signal transduction in cells expressing MCSP on the surface.

MCSP is overexpressed in many human tumors. Thus, the ABMs of the invention are particularly useful in the prevention of tumor formation, eradication of tumors and inhibition of tumor growth. The ABMs of the invention can be used to treat any tumor expressing MCSP. Particular malignancies that can be treated with the ABMs of the invention include, but are not limited to, melanoma and tumor angiogenesis. In one embodiment, the ABMs of the invention are coadministered with an anti-VEGF antibody or another anti-angiogenic antibody to prevent, inhibit or otherwise treat tumor angiogenesis.

The ABMs of the present can be used alone to target and kill tumor cells in vivo. The ABMs can also be used in conjunction with an appropriate therapeutic agent to treat human carcinoma. For example, the ABMs can be used in combination with standard or conventional treatment methods such as chemotherapy, radiation therapy or can be conjugated or linked to a therapeutic drug, or toxin, as well as to a lymphokine or a tumor-inhibitory growth factor, for delivery of the therapeutic agent to the site of the carcinoma. The conjugates of the ABMs of this invention that are of prime importance are (1) immunotoxins (conjugates of the ABM and a cytotoxic moiety) and (2) labeled (e.g. radiolabeled, enzyme-labeled, or fluorochrome-labeled) ABMs in which the label provides a means for identifying immune complexes that include the labeled ABM. The ABMs can also be used to induce lysis through the natural complement process, and to interact with antibody dependent cytotoxic cells normally present.

The cytotoxic moiety of the immunotoxin may be a cytotoxic drug or an enzymatically active toxin of bacterial or plant origin, or an enzymatically active fragment (“A chain”) of such a toxin. Enzymatically active toxins and fragments thereof used are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the ABMs are conjugated to small molecule anticancer drugs. Conjugates of the ABM and such cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as bis(p-azidobenzoyl)hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin may be joined to the Fab fragment of the ABMs. Additional appropriate toxins are known in the art, as evidenced in e.g., published U.S. Patent Application No. 2002/0128448, incorporated herein by reference in its entirety.

In one embodiment, a chimeric, glycoengineered ABM having substantially the same binding specificity of the murine 225.28S monoclonal antibody, is conjugated to ricin A chain. Most advantageously, the ricin A chain is deglycosylated and produced through recombinant means. An advantageous method of making the ricin immunotoxin is described in Vitetta et al., Science 238, 1098 (1987), hereby incorporated by reference.

When used to kill human cancer cells in vitro for diagnostic purposes, the conjugates will typically be added to the cell culture medium at a concentration of at least about 10 nM. The formulation and mode of administration for in vitro use are not critical. Aqueous formulations that are compatible with the culture or perfusion medium will normally be used. Cytotoxicity may be read by conventional techniques to determine the presence or degree of cancer.

As discussed above, a cytotoxic radiopharmaceutical for treating cancer may be made by conjugating a radioactive isotope (e.g., I, Y, Pr) to a chimeric, glycoengineered ABM having substantially the same binding specificity of the murine monoclonal antibody. The term “cytotoxic moiety” as used herein is intended to include such isotopes.

In another embodiment, liposomes are filled with a cytotoxic drug and the liposomes are coated with the ABMs of the present invention. Because there are many MCSP molecules on the surface of the MCSP-expressing malignant cell, this method permits delivery of large amounts of drug to the correct cell type.

Techniques for conjugating such therapeutic agents to antibodies are well known (see, e.g., Arnon et al., “Monoclonal Antibodies for Immunotargeting of Drugs in Cancer Therapy” , in Monoclonal Antibodies and Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp.623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

Still other therapeutic applications for the ABMs of the invention include conjugation or linkage, e.g., by recombinant DNA techniques, to an enzyme capable of converting a prodrug into a cytotoxic drug and the use of that antibody-enzyme conjugate in combination with the prodrug to convert the prodrug to a cytotoxic agent at the tumor site (see, e.g., Senter et al., “Anti-Tumor Effects of Antibody-alkaline Phosphatase”, Proc. Natl. Acad. Sci. USA 85:4842-46 (1988); “Enhancement of the in vitro and in vivo Antitumor Activities of Phosphorylated Mitocycin C and Etoposide Derivatives by Monoclonal Antibody-Alkaline Phosphatase Conjugates”, Cancer Research 49:5789-5792 (1989); and Senter, “Activation of Prodrugs by Antibody-Enzyme Conjugates: A New Approach to Cancer Therapy,” FASEB J. 4:188-193 (1990)).

Still another therapeutic use for the ABMs of the invention involves use, either unconjugated, in the presence of complement, or as part of an antibody-drug or antibody-toxin conjugate, to remove tumor cells from the bone marrow of cancer patients. According to this approach, autologous bone marrow may be purged ex vivo by treatment with the antibody and the marrow infused back into the patient [see, e.g., Ramsay et al., “Bone Marrow Purging Using Monoclonal Antibodies”, J. Clin. Immunol., 8(2):81-88 (1988)].

Furthermore, it is contemplated that the invention comprises a single-chain immunotoxin comprising antigen binding domains that allow substantially the same specificity of binding as the murine 225.28S monoclonal antibody (e.g., polypeptides comprising the CDRs of the murine 225.28S monoclonal antibody) and further comprising a toxin polypeptide. The single-chain immunotoxins of the invention may be used to treat human carcinoma in vivo.

Similarly, a fusion protein comprising at least the antigen-bindingregion of an ABM of the invention joined to at least a functionally active portion of a second protein having anti-tumor activity, e.g., a lymphokine or oncostatin, can be used to treat human carcinoma in vivo.

The present invention provides a method for selectively killing tumor cells expressing MCSP. This method comprises reacting the immunoconjugate (e.g., the immunotoxin) of the invention with said tumor cells. These tumor cells may be from a human carcinoma.

Additionally, this invention provides a method of treating carcinomas (for example, human carcinomas) in vivo. This method comprises administering to a subject a pharmaceutically effective amount of a composition containing at least one of the immunoconjugates (e.g., the immunotoxin) of the invention.

In a further aspect, the invention is directed to an improved method for treating cell proliferation disorders wherein MCSP is expressed, particularly wherein MCSP is abnormally expressed (e.g. overexpressed), including melanoma, comprising administering a therapeutically effective amount of an ABM of the present invention to a human subject in need thereof. Moreover, because MCSP is expressed on activated and inactive pericytes, the ABMs of the invention can be used to treat angiogenesis in any tumor that induces neovascularization. Since pericytes make contact and give support to endothelial cells and also may stabilize new blood vessels, their targeting would inhibit the tumor induced angiogenesis. Ozerdem & Stallcup, Angiogenesis 7(3):269-76 (2004); Erber et al., FASEB J. 18(2):338-40 (February 2004); Grako et al., J. Cell Sci. 112:905-915 (1999); Iivanainen et al., FASEB J. 17:1609-1621 (2003). In a preferred embodiment, the ABM is a glycoengineered anti-MCSP antibody with a binding specificity substantially the same as that of the murine 225.28S monoclonal antibody. In another preferred embodiment the antibody is humanized. Examples of cell proliferation disorders that can be treated by an ABM of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system.

Similarly, other cell proliferation disorders can also be treated by the ABMs of the present invention. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

In accordance with the practice of this invention, the subject may be a human, equine, porcine, bovine, murine, canine, feline, and avian subjects. Other warm blooded animals are also included in this invention.

The subject invention further provides methods for inhibiting the growth of human tumor cells, treating a tumor in a subject, and treating a proliferative type disease in a subject. These methods comprise administering to the subject an effective amount of an ABM composition of the invention.

The invention is further directed to methods for treating non-malignant diseases or disorders in a mammal characterized by abnormal activation or production of MCSP or one or more MCSP ligands, comprising administering to the mammal a therapeutically effective amount of the ABMs of the invention. The subject will generally have MCSP-expressing cells, for instance in diseased tissue thereof, such that the ABMs of the invention are able to bind to cells within the subject.

Abnormal activation or expression of MCSP or a MCSP ligand may be occurring in cells of the subject, e.g. in diseased tissue of the subject. Abnormal activation of MCSP may be attributable to amplification, overexpression or aberrant production of the MCSP and/or MCSP ligand. In one embodiment of the invention, a diagnostic or prognostic assay will be performed to determine whether abnormal production or activation of MCSP (or MCSP ligand) is occurring the subject. For example, gene amplification and/or overexpression of MCSP and/or ligand may be determined. Various assays for determining such amplification/overexpression are available in the art and include the IHC, FISH and shed antigen assays described above. Alternatively, or additionally, levels of an MCSP ligand in or associated with the sample may be determined according to known procedures. Such assays may detect protein and/or nucleic acid encoding it in the sample to be tested. In one embodiment, MCSP ligand levels in a sample may be determined using immunohistochemistry (IHC); see, for example, Scher et al. Clin. Cancer Research 1:545-550 (1995). Alternatively, or additionally, one may evaluate levels of MCSP-encoding nucleic acid in the sample to be tested; e.g. via FISH, southern blotting, or PCR techniques.

Moreover, MCSP or MCSP ligand overexpression or amplification may be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

It is apparent, therefore, that the present invention encompasses pharmaceutical compositions, combinations and methods for treating human malignancies such as melanomas and cancers of the bladder, brain, head and neck, pancreas, lung, breast, ovary, colon, prostate, and kidney. For example, the invention includes pharmaceutical compositions for use in the treatment of human malignancies comprising a pharmaceutically effective amount of an antibody of the present invention and a pharmaceutically acceptable carrier.

The ABM compositions of the invention can be administered using conventional modes of administration including, but not limited to, intravenous, intraperitoneal, oral, intralymphatic or administration directly into the tumor. Intravenous administration is preferred.

In one aspect of the invention, therapeutic formulations containing the ABMs of the invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The ABMs of the present invention may be administered to a subject to treat a disease or disorder characterized by abnormal MCSP or MCSP ligand activity, such as a tumor, either alone or in combination therapy with, for example, a chemotherapeutic agent and/or radiation therapy. Suitable chemotherapeutic agents include cisplatin, doxorubicin, topotecan, paclitaxel, vinblastine, carboplatin, and etoposide

Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g., one which binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, micro emulsions, nano-particles and nanocapsules) or in macroemulsions. Such techniques are disclosed in Remington's Pharmaceutical Sciences. 16th edition, Osol, A. Ed. (1980).

Sustained-release preparations may be prepared. Suitable examples of sustained-release preparations include semipermeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and yethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The compositions of the invention may be in a variety of dosage forms which include, but are not limited to, liquid solutions or suspension, tablets, pills, powders, suppositories, polymeric microcapsules or microvesicles, liposomes, and injectable or infusible solutions. The preferred form depends upon the mode of administration and the therapeutic application.

The compositions of the invention also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The most effective mode of administration and dosage regimen for the pharmaceutical compositions of this invention depends upon the severity and course of the disease, the patient's health and response to treatment and the judgment of the treating physician. Accordingly, the dosages of the compositions should be titrated to the individual patient. Nevertheless, an effective dose of the compositions of this invention will generally be in the range of from about 0.01 to about 2000 mg/kg.

The molecules described herein may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The dosages of the present invention may, in some cases, be determined by the use of biomarkers. Biomarkers are molecular markers that are used to assess pharmacodynamics of a therapeutic and determine which subjects are most likely to respond. For example, biomarkers for anti-MCSP therapy may be molecules (e.g., focal adhesion kinase (FAK), extracellular signal-regulated kinase (ERK), or others) that are in the MCSP downstream signaling pathway that leads to a cell proliferation disorder. Thus, biomarkers may be used to determine in what amount to administer the ABM of the present invention. The present invention also provides for a method of administering an amount of an ABM to a patient by first determining the expression of biomarkers of disorders marked by MCSP expression.

The dosages of the present invention may, in some cases, be determined by the use of predictive biomarkers. Predictive biomarkers are molecular markers that are used to determine (i.e., observe and/or quantitate) a pattern of expression and/or activation of tumor related genes or proteins, or cellular components of a tumor related signaling pathway. Elucidating the biological effects of targeted-therapies in tumor tissue and correlating these effects with clinical response helps identify the predominant growth and survival pathways operative in tumors, thereby establishing a profile of likely responders and conversely providing a rational for designing strategies to overcoming resistance. For example, biomarkers for anti-MCSP therapy may comprise molecules that are in the MCSP downstream signaling pathway that leads to a cell proliferation disorder including, but not limited to: FAK, ERK, membrane-type 3 matrix metalloproteinase (MT3-MMP), Cdc42, Ack-1, and p130cas. Yang et al., J. Cell Biol. 165(6):881-891 (June 2004); lida et al., J. Biol. Chem. 276(22):18786-18794 (2001); Eisenmann et al., Nat. Cell Biol. 1(8):507-513 (1999).

Predictive biomarkers may be measured by cellular assays that are well known in the art including, but not limited to immunohistochemistry, flow cytometry, immunofluorescence, capture-and-detection assays, and reversed phase assays, and/or assays set forth in U.S. Pat. Appl. Pub. No. 2004/0132097 Al, the entire contents of which are herein incorporated by reference. Predictive biomarkers of anti-MCSP therapy, themselves, can be identified according to the techniques set forth in U.S. Pat. Appl. Pub. No. 2003/0190689A1, the entire contents of which are hereby incorporated by reference.

In one aspect, the present invention provides for a method for treating an MCSP-related disorder comprising predicting a response to anti-MCSP therapy in a human subject in need of treatment by assaying a sample from the human subject prior to therapy with one or a plurality of reagents that detect expression and/or activation of predictive biomarkers for an MCSP-related disorder such as cancer; determining a pattern of expression and/or activation of one or more of the predictive biomarkers, wherein the pattern predicts the human subject's response to the anti-MCSP therapy; and administering to a human subject who is predicted to respond positively to anti-MCSP treatment a therapeutically effective amount of a composition comprising an ABM of the present invention. As used herein, a human subject who is predicted to respond positively to anti-MCSP treatment is one for whom anti-MCSP will have a measurable effect on the MCSP-related disorder (e.g., tumor regression/shrinkage) and for whom the benefits of anti-MCSP therapy are not outweighed by adverse effects (e.g., toxicity). As used herein, a sample means any biological sample from an organism, particularly a human, comprising one or more cells, including. single cells of any origin, tissue or biopsy samples which has been removed from organs such as breast, lung, gastrointestinal tract, skin, cervix, ovary, prostate, kidney, brain, head and neck, or any other organ or tissue of the body, and other body samples including, but not limited to, smears, sputum, secretions, cerebrospinal fluid, bile, blood, lymph fluid, urine and feces.

The composition comprising an ABM of the present invention will be formulated, dosed, and administered in a fashion consistent with good medical practice. Factors for consideration in this context include the particular disease or disorder being treated, the particular mammal being treated, the clinic condition of the individual patient, the cause of the disease or disorder, the site of delivery of the agent, the method of administration, the scheduling of administration, and other factors known to medical practitioners. The therapeutically effective amount of the antagonist to be administered will be governed by such considerations.

As a general proposition, the therapeutically effective amount of the antibody administered parenterally per dose will be in the range of about 0.1 to 20 mg/kg of patient body weight per day, with the typical initial range of antagonist used being in the range of about 2 to 10 mg/kg.

In a preferred embodiment, the ABM is an antibody, preferably a humanized antibody. Suitable dosages for such an unconjugated antibody are, for example, in the range from about 20 mg/m² to about 1000 mg/m². For example, one may administer to the patient one or more doses of substantially less than 375 mg/m² of the antibody, e.g., where the dose is in the range from about 20 mg/m² to about 250 mg/m², for example from about 50 mg/m² to about 200 mg/m².

Moreover, one may administer one or more initial dose(s) of the antibody followed by one or more subsequent dose(s), wherein the mg/m² dose of the antibody in the subsequent dose(s) exceeds the mg/m² dose of the antibody in the initial dose(s). For example, the initial dose may be in the range from about 20 mg/m² to about 250 mg/m² (e.g., from about 50 mg/m² to about 200mg/m²) and the subsequent dose may be in the range from about 250 mg/m² to about 1000 mg/m².

As noted above, however, these suggested amounts of ABM are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

In the case of anti-MCSP antibodies used to treat tumors, optimum therapeutic results are generally achieved with a dose that is sufficient to completely saturate the MCSP molecule on the target cells. The dose necessary to achieve saturation will depend on the number of MCSP molecules expressed per tumor cell (which can vary significantly between different tumor types). Serum concentrations as low as 30 nM may be effective in treating some tumors, while concentrations above 100 nM may be necessary to achieve optimum therapeutic effect with other tumors. The dose necessary to achieve saturation for a given tumor can be readily determined in vitro by radioimmunoassay or immunoprecipiation.

In general, for combination therapy with radiation, one suitable therapeutic regimen involves eight weekly infusions of an anti-MCSP ABM of the invention at a loading dose of 100-500 mg/m² followed by maintenance doses at 100-250 mg/m² and radiation in the amount of 70.0 Gy at a dose of 2.0 Gy daily. For combination therapy with chemotherapy, one suitable therapeutic regimen involves administering an anti-MCSP ABM of the invention as loading/maintenance doses weekly of 100/100 mg/m², 400/250 mg/m², or 500/250 mg/m² in combination with cisplatin at a dose of 100 mg/m² every three weeks. Alternatively, gemcitabine or irinotecan can be used in place of cisplatin.

The ABM of the present invention is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

One may administer other compounds, such as cytotoxic agents, chemotherapeutic agents, immunosuppressive agents and/or cytokines with the antagonists herein. The combined administration includes coadministration, using separate formulations or a single pharmaceutical formulation, and consecutive administration in either order, wherein preferably there is a time period while both (or all) active agents simultaneously exert their biological activities.

It would be clear that the dose of the composition of the invention required to achieve cures may be further reduced with schedule optimization.

In accordance with the practice of the invention, the pharmaceutical carrier may be a lipid carrier. The lipid carrier may be a phospholipid. Further, the lipid carrier may be a fatty acid. Also, the lipid carrier may be a detergent. As used herein, a detergent is any substance that alters the surface tension of a liquid, generally lowering it.

In one example of the invention, the detergent may be a nonionic detergent. Examples of nonionic detergents include, but are not limited to, polysorbate 80 (also known as Tween 80 or (polyoxyethylenesorbitan monooleate), Brij, and Triton (for example Triton WR-1339 and Triton A-20).

Alternatively, the detergent may be an ionic detergent. An example of an ionic detergent includes, but is not limited to, alkyltrimethylammonium bromide.

Additionally, in accordance with the invention, the lipid carrier may be a liposome. As used in this application, a “liposome” is any membrane bound vesicle which contains any molecules of the invention or combinations thereof.

Articles of Manufacture

In another embodiment of the invention, an article of manufacture containing materials useful for the treatment of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is an anti-MCSP antibody. The label or package insert indicates that the composition is used for treating the condition of choice, such as a non-malignant disease or disorder, where the disease or disorder involves abnormal activation or production of MCSP and/or a MCSP-ligand, for example a benign hyperproliferative disease or disorder. Moreover, the article of manufacture may comprise (a) a first container with a composition contained therein, wherein the composition comprises a first antibody which binds MCSP and inhibits growth of cells which overexpress MCSP; and (b) a second container with a composition contained therein, wherein the composition comprises a second antibody which binds MCSP and blocks ligand activation of an MCSP receptor. The article of manufacture in this embodiment of the invention may further comprises a package insert indicating that the first and second antibody compositions can be used to treat a non-malignant disease or disorder from the list of such diseases or disorders in the definition section above. Moreover, the package insert may instruct the user of the composition (comprising an antibody which binds MCSP and blocks ligand activation of an MCSP receptor) to combine therapy with the antibody and any of the adjunct therapies described in the preceding section (e.g. a chemotherapeutic agent, an MCSP-targeted drug, an anti-angiogenic agent, an immumosuppressive agent, tyrosine kinase inhibitor, an anti-hormonal compound, a cardioprotectant and/or a cytokine). Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

All patents, applications, and publications cited in this application are hereby incorporated by reference in their entirety.

### EXAMPLES

Unless otherwise specified, references to the numbering of specific amino acid residue positions in the following Examples are according to the Kabat numbering system. Except where otherwise noted, the materials and methods used to make the antigen binding molecules in these working examples are in accordance with those set forth in the examples of U.S. patent application Ser. No. 10/981,738, which is hereby incorporated by reference in its entirety.

### Example 1

### Generation of Humanized Anti-MCSP MAbs

A. High Homology Acceptor Approach

Under this approach, a high homology antibody acceptor framework search was performed by aligning the parental protein sequence, derived from the mouse derived scFv antibody 225.28S to a collection of human germ-line sequences and picking that human sequence that showed the highest sequence identity while at the same time conserving all canonical residues on a functional level. Here, the sequences IGHV3-15 (Acc. No. X92216) and IGHV3-7 (Acc. No. M99649) from the IMGT database were taken as the framework acceptor sequences. Both are members of the VH3 family. The IGKV1-9 sequence (Acc. No. Z00013) from the VK1 family of the same database was chosen to be the framework acceptor for the light chain. On these three acceptor frameworks the three complementary determining regions (CDRs) of each of the murine 225.28S heavy and light variable domains were grafted. Since the framework 4 region (FR4) is not part of the variable region of the germ line gene, the alignment for that position was done individually. The JH6 region was chosen for the heavy chain, and the JK4 region was chosen for the light chain. Molecular modelling of the designed immunoglobulin domain revealed some positions potentially requiring the murine amino acid residues instead of the human ones outside of the CDR regions. Re-introducing murine amino acid residues into the human framework would generate the so-called back mutations. For example, the human acceptor amino acid residue at Kabat position 94 (Threonine in IGHV3-15) was back mutated to a Serine residue in one of the variants. To prove the hypothesis of necessitating these back mutations, humanized antibody variants were designed that either included or omitted the back mutations.

In the sequence of the 225.28S light chain, a statistical analysis revealed a whole stretch of “rare” residues in FR2. These are Glu45, Pro46, Leu48, and Phe49. Analysis of the 3D molecular model showed that all of these residues make strong interactions towards the CDRs of the light chain, and (apart from Leu48) also to the CDR3 of VH. In this modelling step, it was also found that Pro46 makes important contacts to the VH CDR3. To investigate the importance of these residues, they were incorporated as back mutations into the humanized VL constructs. The humanized constructs containing back mutations were compared to humanized constructs prepared in accordance with the “mixed framework” approach discussed below for their ability to bind antigen.

B. Mixed Framework Approach

In order to avoid introducing back mutations at critical positions (critical to retain good antigen binding affinity or antibody functions) in the human acceptor framework, it was investigated whether either the framework region 1 (FR1), the framework regions 1 (FR1) and 2 (FR2) together, or the FR3 of a functionally humanized antibody could be replaced by human antibody sequences already having donor residues, or functionally equivalent ones, at those important positions in the natural human germline sequence. For this purpose, the VH frameworks FR1, FR2 and FR3 of the murine 225.28S VH sequence were aligned individually to human germ-line sequences. Here, highest sequence identity was not important, and was not used for choosing acceptor frameworks, but instead matching of several critical residues was assumed to be more important. Those critical residues comprise the so-called canonical residues, and also those residues at positions 27, 28, and 30 (Kabat numbering), which lie outside of the CDR1 definition by Kabat, but inside of the CDR1 as defined by Kabat, and often are involved in antigen binding. In addition, critical residues are those which show important interaction towards the CDRs, as can be determined using molecular modelling. The IMGT sequences IGHV1-58 (Accession No. M29809), and IGHV1-46 (Accession No. X92343) were chosen as suitable candidates for replacing either FR1, FR2, or FR3. In brief, IGHV 1-46 was used as an acceptor for all frameworks, thus generating a single framework acceptor, which is identical to the donor FR regions to 53% at the amino acid level. IGHV1-46 was also used as the FR1 and FR2 acceptor, while IGHV1-58 was used for FR3. Also the IGHV3-7 was used as FR1 and FR2 acceptor, and the IGHV3-15 was used for FR1, FR2, and/or FR3. The rationale for mixing the IGHV3-7 with the IGHV3-15 was to have optimal homology in FR1 and FR2, and having matching residues at Kabat positions 71 and 94. In all these constructs the JH6 was used for the FR4 region.

As mentioned above with respect to the high homology approach, the FR2 region of the humanized light chain would require some effort. We introduced several FR2 regions of other germ line antibodies, namely the IGKV2-28 (Acc. No. X63397) and the IGKV2D-30 (Acc. No. X63402). Obviously, “rare” residues are found rarely in the human germ line repertoire. So, the FR2 of anon-germ line antibody (Gen Bank Acc. No. AAA17574), that is derived from human peripheral B-cells and incorporates the Proline 46 residue, was also included in the acceptor FR collection.

To scrutinize the idea of removing “rare” residues in the light chain constructs, that are within the CDR regions, the variants M-KV10, M-KV11, and M-KV12 were constructed. All of them start from the M-KV9 design. M-KV10 replaces the murine Lys24 by a human Arginine, and also replaces the Val33 by a human Leucine. M-KV11 replaces only the murine Val33 by a human Leucine. M-KV12 replaces the three-amino acid stretch Arg-Tyr-Thr (54 to 56) by the human VK1 derived Leu-Gln-Ser tri-peptide.

After having designed the protein sequences, DNA sequences encoding these proteins were synthesized as detailed below. Using this approach back mutations could be avoided in most of the constructs of the heavy chain, in order to retain good levels of antigen binding.

The chronology and the reasoning of the mixed framework constructs is explained in the results section.

C. Synthesis of the Antibody Genes

After having designed the amino acid sequence of the humanized antibody V region, the DNA sequence had to be generated. The DNA sequence data of the individual framework regions was found in the databases (e.g. the International Immunogenetics Information System maintained by the European Bioinformatics Institute, http://imgt.cines.fr) for human germ line sequences. The DNA sequence information of the CDR regions was deduced from the published protein sequence of the murine 225.28S antibody. Neri et al., J. Invest. Dermatol. 107(2):164-170 (1996). With these sequences, the whole DNA sequence was virtually assembled. Having this DNA sequence data, diagnostic restriction sites were introduced in the virtual sequence, by introducing silent mutations, creating recognition sites for restriction endonucleases. To obtain the physical DNA chain, gene synthesis was performed (e.g. Wheeler et al. 1995). In this method, oligonucleotides are designed from the genes of interest, such, that a series of oligonucleotides is derived from the coding strand, and one other series is from the non-coding strand. The 3′ and 5′ ends of each oligonucleotide (except the very first and last in the row) always show complementary sequences to two primers derived from the opposite strand. When putting these oligonucleotides into a reaction buffer suitable for any heat stable polymerase, and adding Mg²⁺, dNTPs and a DNA polymerase, each oligonucleotide is extended from its 3′ end. The newly formed 3′ end of one primer then anneals with the next primer of the opposite strand, and extending its sequence further under conditions suitable for template dependant DNA chain elongation. The final product was cloned into a conventional vector for propagation in E. coli.

D. Antibody Production

Human heavy (SEQ ID NO:25) and light chain (SEQ ID NO:53) leader sequences (for secretion) were added upstream of the above variable region DNA sequences. Downstream of the variable regions, the constant region of human IgG1 for the heavy chain and the human kappa constant region for the light chain, respectively, were added using standard molecular biology techniques. The resulting full-length humanized antibody heavy and light chain DNA sequences were subcloned into mammalian expression vectors (one for the light chain and one for the heavy chain) under the control of the MPSV promoter and upstream of a synthetic polyA site, each vector carrying an EBV OriP sequence

Antibodies were produced by co-transfecting HEK293-EBNA cells with the mammalian antibody heavy and light chain expression vectors using a calcium phosphate-transfection approach. Exponentially growing HEK293-EBNA cells were transfected by the calcium phosphate method. Cells were grown as adherent monolayer cultures in T flasks using DMEM culture medium supplemented with 10% FCS, and were transfected when they were between 50 and 80% confluent. For the transfection of a T75 flask, 8 million cells were seeded 24 hours before transfection in 14 ml DMEM culture medium supplemented with FCS (at 10% V/V final), 250 μg/ml neomycin, and cells were placed at 37° C. in an incubator with a 5% CO₂ atmosphere overnight. For each T75 flask to be transfected, a solution of DNA, CaCl₂ and water was prepared by mixing 47 μg total plasmid vector DNA divided equally between the light and heavy chain expression vectors, 235 μl of a 1M CaCl₂ solution, and adding water to a final volume of 469 μl. To this solution, 469 μl of a 50mM HEPES, 280 mM NaCl, 1.5 mM Na₂HPO₄ solution at pH 7.05 were added, mixed immediately for 10 sec and left to stand at room temperature for 20 sec. The suspension was diluted with 12 ml of DMEM supplemented with 2% FCS, and added to the T75 in place of the existing medium. The cells were incubated at 37° C., 5% CO₂ for about 17 to 20 hours, then medium was replaced with 12 ml DMEM, 10% FCS. The conditioned culture medium was harvested 5 to 7 days post-transfection centrifuged for 5 min at 1200 rpm, followed by a second centrifugation for 10 min at 4000 rpm and kept at 4° C. The secreted antibodies were purified by Protein A affinity chromatography, followed by cation exchange chromatography and a final size exclusion chromatographic step on a Superdex 200 column (Amersham Pharmacia) exchanging the buffer to phosphate buffer saline and collecting the pure monomeric IgG1 antibodies. Antibody concentration was estimated using a spectrophotometer from the absorbance at 280 nm. The antibodies were formulated in a 25 mM potassium phosphate, 125 mM sodium chloride, 100 mM glycine solution of pH 6.7.

E. Glycoengineering of Humanized Antibodies

Glycoengineering of the humanized variants was performed by co-transfection into mammalian cells of the antibody expression vectors together with a GnT-III glycosyltransferase expression vector, or together with a GnT-III expression vector plus a Golgi mannosidase II expression vector. The polypeptide having GnTIII activity is a fusion polypeptide comprising the Golgi localization domain of a heterologous Golgi resident polypeptide prepared according to the methods taught in U.S. Pat. Appl. Publ. No. 20040241817 A1, the contents of which are hereby incorporated by reference in their entirety. Glycoengineered antibodies were purified and formulated as described above for the non-glycoengineered antibodies. The oligosaccharides attached to the Fc region of the antibodies were analyzed by MALDI/TOF-MS as described below. The glycoengineering methodology that can be used with the ABMs of the present invention has been described in greater detail in U.S. Pat. No. 6,602,684 and Provisional U.S. Patent Application No. 60/441,307 and WO 2004/065540, the entire contents of each of which is incorporated herein by reference in its entirety. The glycoengineered ABMs of the present invention have reduced amounts of fucose residues in the Fc region. The ABMs of the present invention can also be glycoengineered to have reduced fucose residues in the Fc region according to the techniques disclosed in EP 1 176 195 A1, the entire contents of which are incorporated by reference herein.

### EXAMPLE 2

### Materials and Methods

A. Oligosaccharide Analysis

1. Oligosaccharide Release Method for Antibodies in Solution

Between 40 and 50 μg of antibody were mixed with 2.5 mU of PNGaseF (Glyko, U.S.A.) in 2 mM Tris, pH7.0 in a final volume of 25 microliters, and the mix was incubated for 3 hours at 37° C.

2. Sample Preparation for MALDF/TOF-MS

The enzymatic digests containing the released oligosaccharides were incubated for a further 3 h at room temperature after the addition of acetic acid to a final concentration of 150 mM, and were subsequently passed through 0.6 ml of cation exchange resin (AG50W-X8 resin, hydrogen form, 100-200 mesh, BioRad, Switzerland) packed into a micro-bio-spin chromatography column (BioRad, Switzerland) to remove cations and proteins. One microliter of the resulting sample was applied to a stainless steel target plate, and mixed on the plate with 1 μl of sDHB matrix. sDHB matrix was prepared by dissolving 2 mg of 2,5-dihydroxybenzoic acid plus 0.1 mg of 5-methoxysalicylic acid in 1 ml of ethanol/10 mM aqueous sodium chloride 1:1 (v/v). The samples were air dried, 0.2 μl ethanol was applied, and the samples were finally allowed to re-crystallize under air.

3. MALDI/TOF-MS

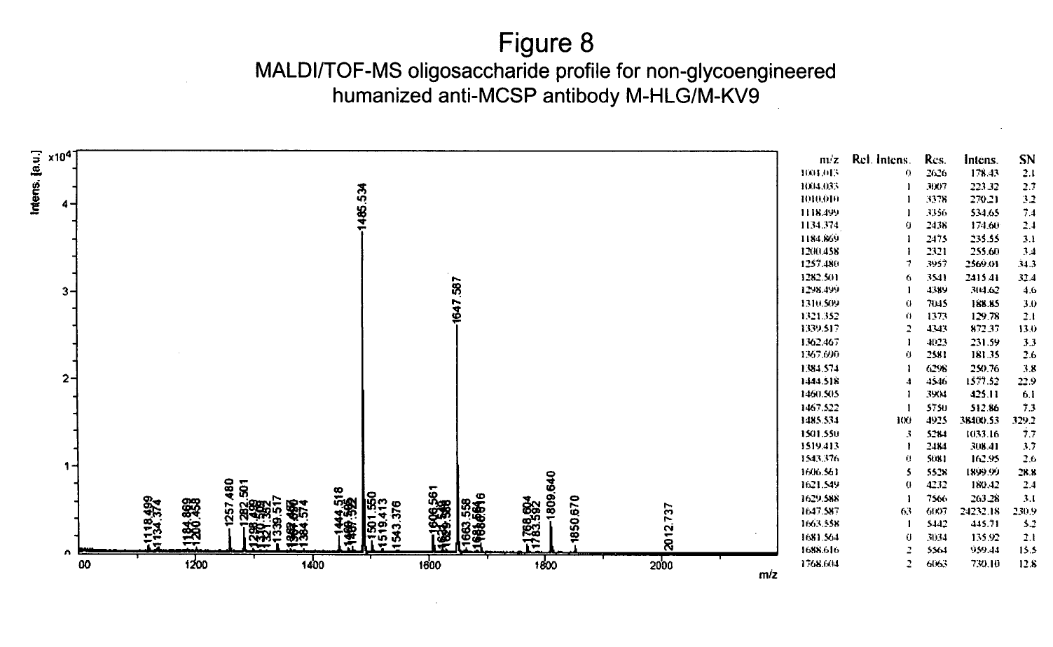

The MALDI-TOF mass spectrometer used to acquire the mass spectra was a Voyager Elite (Perspective Biosystems). The instrument was operated in the linear configuration, with an acceleration of 20 kV and 80 ns delay. External calibration using oligosaccharide standards was used for mass assignment of the ions. The spectra from 200 laser shots were summed to obtain the final spectrum. A typical spectrum is shown in FIG. 8.

B. Antigen Binding Assay

The purified, monomeric humanized antibody variants were tested for binding to the human HMW-MAA/MCSP antigen on the A375 human melanoma cell line, using a flow cytometry-based assay. 200,000 cells (in 180 μl FACS buffer (PBS containing 2% FCS and 5 mM EDTA) were transferred to 5 ml polystyrene tubes and 20 μl 10 fold concentrated anti-MCSP antibody (primary antibody) samples (1-5000 ng/ml final concentration) or PBS only were added. After gently mixing the samples, the tubes were incubated at 4° C. for 30 min in the dark. Subsequently, samples were washed twice with FACS buffer and pelleted at 300 g for 3 min. Supernatant was aspirated off and cells were taken up in 50 μl FACS buffer and 2 μl secondary antibody (anti-Fc-specific F(ab′)2-FITC fragments (Jackson Immuno Research Laboratories, USA)) was added and the tubes were incubated at 4° C. for 30 min. Samples were washed twice with FACS buffer and taken up in 500 μl of FACS buffer for analysis by Flow Cytometry. Binding was determined by plotting the geometric mean fluorescence against the antibody concentrations.

C. Antibody-Dependent Cellular Cytotoxicity Assay

Human peripheral blood mononuclear cells (PBMC) were used as effector cells and were prepared using Histopaque-1077 (Sigma Diagnostics Inc., St. Louis, M063178 USA) and following essentially the manufacturer's instructions. In brief, venous blood was taken with heparinized syringes from healthy volunteers. The blood was diluted 1:0.75-1.3 with PBS (not containing Ca++ or Mg++) and layered on Histopaque-1077. The gradient was centrifuged at 400×g for 30 min at room temperature (RT) without breaks. The interphase containing the PBMC was collected and washed with PBS (50 ml per cells from two gradients) and harvested by centrifugation at 300×g for 10 minutes at RT. After resuspension of the pellet with PBS, the PBMC were counted and washed a second time by centrifugation at 200×g for 10 minutes at RT. The cells were then resuspended in the appropriate medium for the subsequent procedures.

The effector to target ratio used for the ADCC assays was 100:1 and 25:1 for PBMC cells. The effector cells were prepared in AIM-V medium at the appropriate concentration in order to add 50 μl per well of round bottom 96 well plates. One set of target cells were human MCSP expressing cells derived from melanoma patients (e.g., A375, A2058, or SK-Mel5) grown in DMEM containing 10% FCS. Another set of target cells, that should be a model for the pericytes, were human aortic smooth muscle cells, named HuSMC (obtained from Promocell, Heidelberg Germany). HuSMC cells were cultivated in medium supplied by Promocell. Target cells were washed in PBS, counted and resuspended in AIM-V at 0.3 million per ml in order to add 30'000 cells in 100 μl per microwell. Antibodies were diluted in AIM-V, added in 50 μl to the pre-plated target cells and allowed to bind to the targets for 10 minutes at RT. The effector cells then were added and the plate was incubated overnight, and for four hours, for the melanoma cells and the HuSMC, respectively, at 37° C. in a humidified atmosphere containing 5% CO₂. Killing of target cells was assessed by measurement of lactate dehydrogenase (LDH) release from damaged cells using the Cytotoxicity Detection kit (Roche Diagnostics, Rotkreuz, Switzerland). After the 4-hour incubation the plates were centrifuged at 800×g. 100 μl supernatant from each well was transferred to a new transparent flat bottom 96 well plate. 100 μl color substrate buffer from the kit were added per well. The Vmax values of the color reaction were determined in an ELISA reader at 490 nm for at least 10 min using SOFTmax PRO software (Molecular Devices, Sunnyvale, Calif. 94089, USA). Spontaneous LDH release was measured from wells containing only target and effector cells but no antibodies. Maximal release was determined from wells containing only target cells and 1% Triton X-100. Percentage of specific antibody-mediated killing was calculated as follows: ((x−SR)/(MR−SR)*100, where x is the mean of Vmax at a specific antibody concentration, SR is the mean of Vmax of the spontaneous release and MR is the mean of Vmax of the maximal release.

D. Results and Discussion

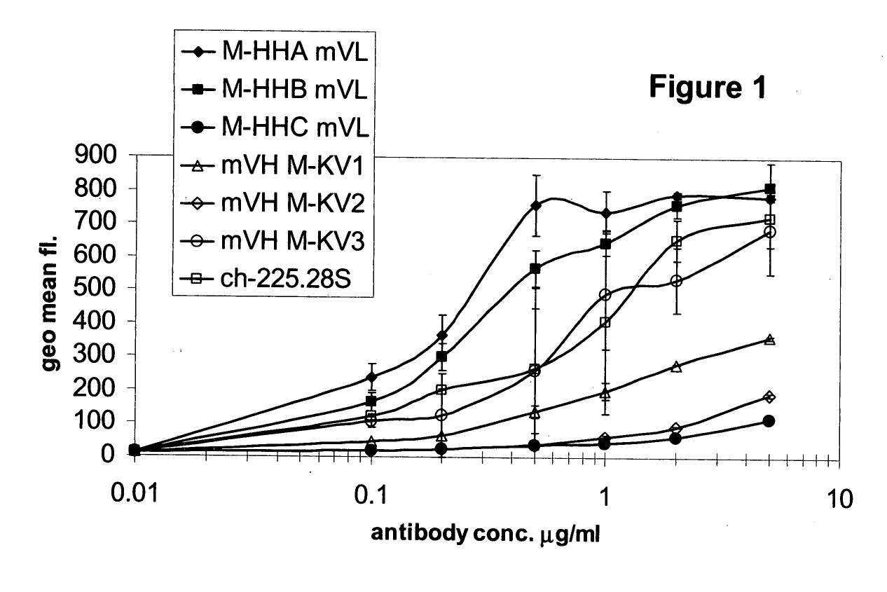

The three initial heavy chain constructs M-HHA, M-HHB, and M-HHC as well as the three initial light chain constructs M-KV1, M-KV2, and M-KV3 were assayed for their binding properties to its cognate antigen, MCSP. For this, the humanized heavy chain constructs were coexpressed with the murine light chain (mVL), and the humanized light chains were coexpressed with the murine heavy chain (mVH). The results are shown in FIG. 1, which shows that the two heavy chain constructs M-HHA and M-HHB, more or less retain their binding properties when combined with the murine VL. In contrast, the construct M-HHC loses its binding potential significantly. M-HHC differs from M-HHB only in the two changes Gly49Ala, and Glu50Asn. Since the Alanine at position 49 is actually the murine one, the Asparagine at position 50 is strictly prohibited. Therefore, the murine Glutamate 50 was kept in all further variants. This means that the IGHV3-15 frame work satisfies all the requirements for canonical and other key residues (Note that position 50 is part of the Kabat CDR2). The humanized light chain constructs M-KV1 and M-KV2 show strongly diminished binding activity compared to its murine counterpart. Whereas the construct M-KV3 shows binding behaviour similar to the murine light chain. This means that the mutations Ile21Val, Leu46Pro, Ile48Leu, and Tyr49Phe restored the binding of the previously inactive variant M-KV2. Either these amino acid residues work synergistically or one single residue is responsible for the whole effect.

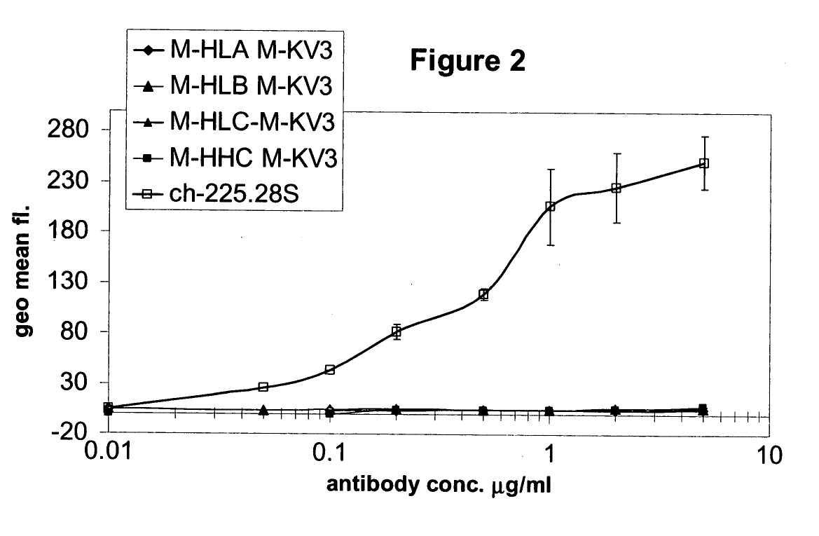

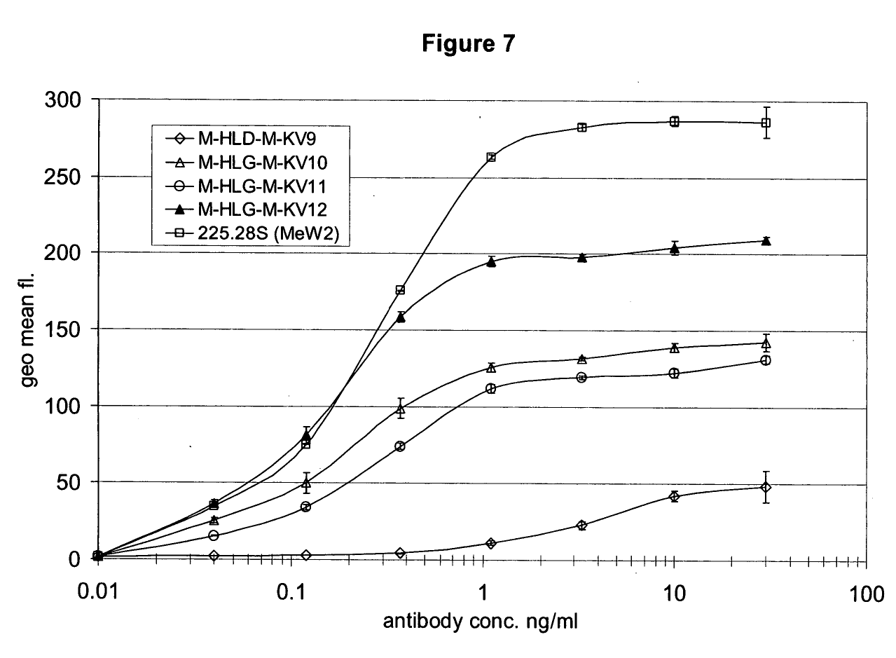

FIG. 2 shows the binding data of the “Low-Homology” constructs M-HLA, M-HLB, and M-HLC combined with the light chain construct M-KV3. Obviously, the binding of these three variants is abolished completely, leading to the conclusion that one or more of the key residues (including the canonicals) are not satisfied in the humanized VH constructs. Since the residues 27 and 30 are expected to be responsible for some “fine tuning” of the binding activity rather than abolishing the binding properties completely, the important residues are expected to be located in framework 3. Two obvious candidates seem to be Thr93, and Ser94 of the 225.28S sequence. If using the IGHV1-58 FR3 sequence, then Ala93 and Ala94 would be the residues putatively responsible for diminishing antigen binding activity. The influence of other FR residues cannot be ruled out, but seemed to be rather improbable based on statistical analysis as well as analysis of the molecular model of the 3D structure of the 225.28S antibody. Support for the importance of residues 27 and 30 is shown in FIG. 7, since the construct M-HLD has regained some residual binding activity, indicating that introduction of residues Phe27 and Ser30 can influence binding behavior.

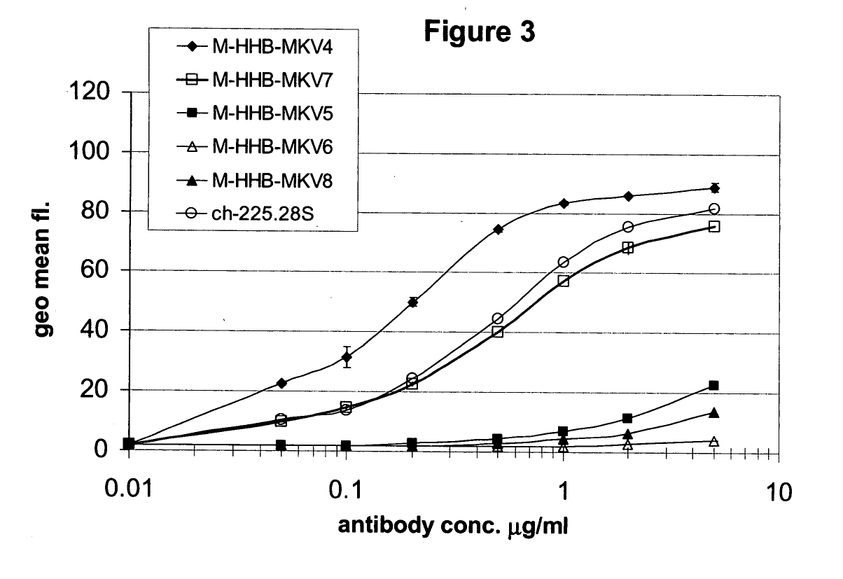

In order to pinpoint the key residues of the light chain, the constructs M-KV4, 5, 6, 7, 8, and 9 were generated. M-KV4 removes one back mutation of M-KV3 (Val21Ile). M-KV5 uses a new FR2 (IGKV2-28; Acc. No. X63397), that has Gln42 and Ser43 occurring naturally, as well as Gln45 that is (to some extent) similar to the murine Glu45. M-KV6 has the IGKV2D-30 (Acc. No. X63402) FR2 sequence. M-KV7 is the Leu46Pro derivative of the FR2 region of M-KV1 (thus introducing one back mutation into the FR2). M-KV8 is the Tyr49Phe variant of the FR2 region of M-KV1 (thus introducing another back mutation into the FR2). The result of the binding data of these light chain constructs, when paired with the M-HHB heavy chain, is depicted in FIG. 3. The construct M-KV4 has gained affinity to its antigen as compared to the ch-225.28S antibody. Constructs M-KV5 and 6 have not regained their functional properties, indicating that the mutations introduced into them were irrelevant. The M-KV7 antibody showed binding properties as good as the ch-225.28S light chain indicating that one single point mutation (Leu46Pro) was necessary and sufficient to recover full binding activity of the previously inactive light chain construct M-KV1.

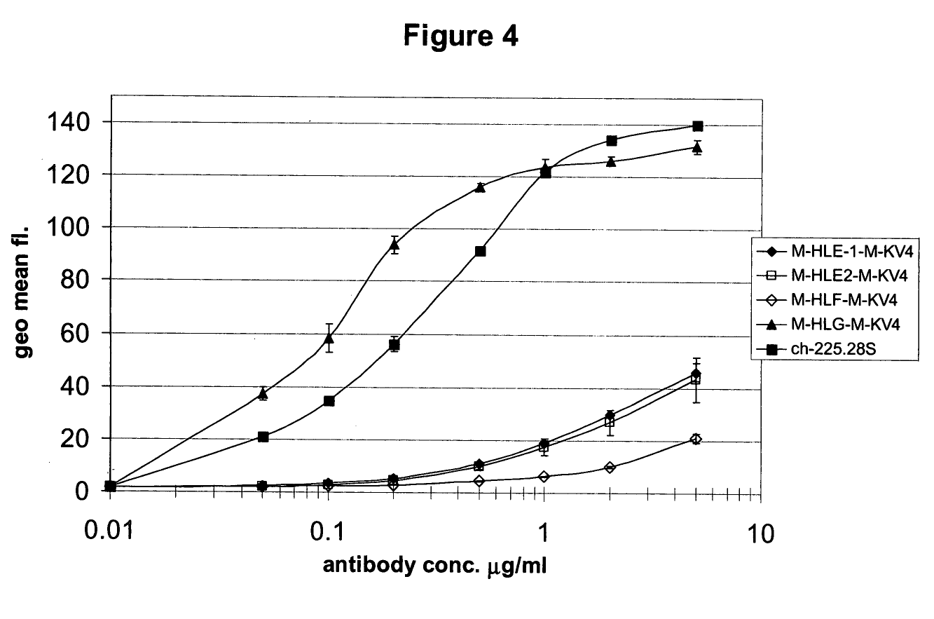

In order to explore the possibility of generating hybrid framework constructs of the humanized heavy chains, we replaced the FR1 and FR2 regions of the M-HLB and M-HLC by that of the IGHV3-15 (Acc. No. X92216) yielding constructs M-HLE1 and M-HLE2. These two constructs differ only in the fact that residues 61 to 64 (which are members of the CDR2 as defined by Kabat, but not as defined by Chothia) are either of human (M-HLE1) or murine (M-HLE2) origin. These constructs would tell us whether the key residues for the failure of constructs M-HLB and C would be located in the FR1 and FR2 area, or as was expected, in the FR3 area. The new constructs would be comprised of framework regions derived from the class 1 and 3 of the VH family. Thus instability could arise, albeit during analysis of the 3D molecular model no obvious sources of this could be identified. Simultaneously, the FR3 of IGHV3-15 (which proved to be functional in the M-HHB construct) was combined with the FR1 and 2 regions of the IGHV3-7 sequence, leading to the constructs M-HLF and M-HLG. M-HLF has its CDR1 completely humanized, and differs from M-HLG only at position 31 and 35. M-HLG has the murine sequence at these positions. FIG. 4 shows the result of the antigen binding experiment when pairing the heavy chain constructs M-HLE1, E2, F, and G with the light chain construct M-KV4. Constructs M-HLE1, and M-HLE2 show some residual binding, indicating some improvement over its predecessor M-HLB and C. Still, this binding is far away from being useful. Construct M-HLF has almost no binding, which could be restored by introducing the two mutations Ser31Asn and Ser35Asn (M-HLG). M-HLG showed, similar to M-HHB an equal or even higher affinity to the antigen than the parental antibody ch-225.28S. This indicates further the importance of some critical residues in FR3 (positions 93 Threonine and 94 Serine, or Threonine as mentioned above). Albeit some importance has to be put on the residues in FR1 and 2. (e.g. Phenylalanine27 or Threonine30) as demonstrated by the M-HLE1 and 2 variants.

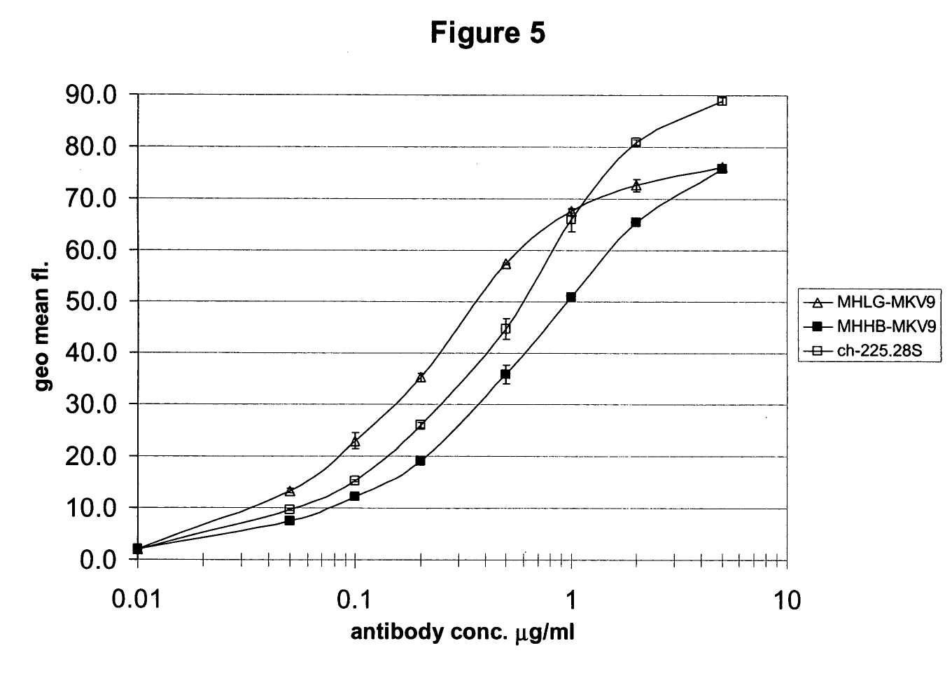

Finally, the light chain variant M-KV9 was generated by introducing the FR2 of a non-germ line antibody (Gen Bank Acc. No. AAA17574) into the M-KV2 construct. This acceptor FR was derived from human peripheral B-cells (Weber et al. J Clin Invest. 93(5):2093-2105. (1994)). This antibody is rearranged and derived from the VK3 family. This light chain was coexpressed, either with the M-HHB, or with the M-HLG heavy chain and assayed for binding function. The result of this is shown in FIG. 5. M-KV9, together with M-HLG shows excellent binding properties, combined with M-HHB, M-KV9 also shows good binding data, albeit slightly reduced compared to the ch-225.28S. FIG. 7 shows that the removal of rare residues within the CDR1 and CDR2 of the light chain is not feasible, since the constructs M-KV10 to 12 all showed reduced antigen binding activity.

E. Analysis of “Rare” Residues

By definition, “rare” residues are those that occur with a frequency of equal to or less than 1% in the corresponding ensemble of germ line sequences.

In the 225.28S VH sequence two “rare” residues are found: Glycine 88 and Serine 94. Glycine 88 can be replaced by the “frequent” human residue Alanine. Serine 94, apparently can be replaced by Threonine, but not by Arginine or Alanine. Tabulated data on canonical residues would predict that Alanine and Arginine at Kabat position 94 would lead to the same canonical loop conformation in CDRI as when Serine is present (http://www.rubic.rdg.ac.uk/abeng/canonicals.html). This view takes into account only the canonical loop structure, but not the potential involvement in antigen binding observed for several canonical residues; for example at position 94 (see analysis of canonicals).

F. Results of the ADCC Experiments

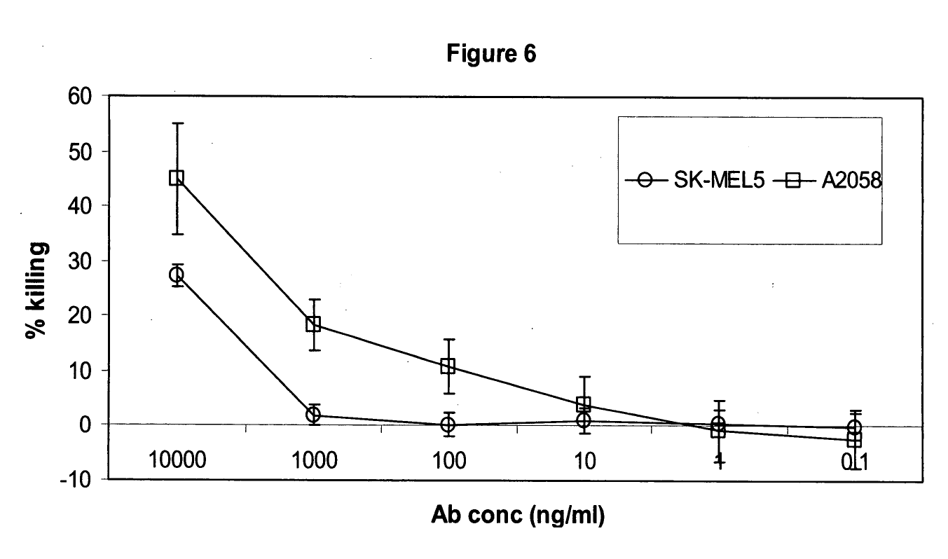

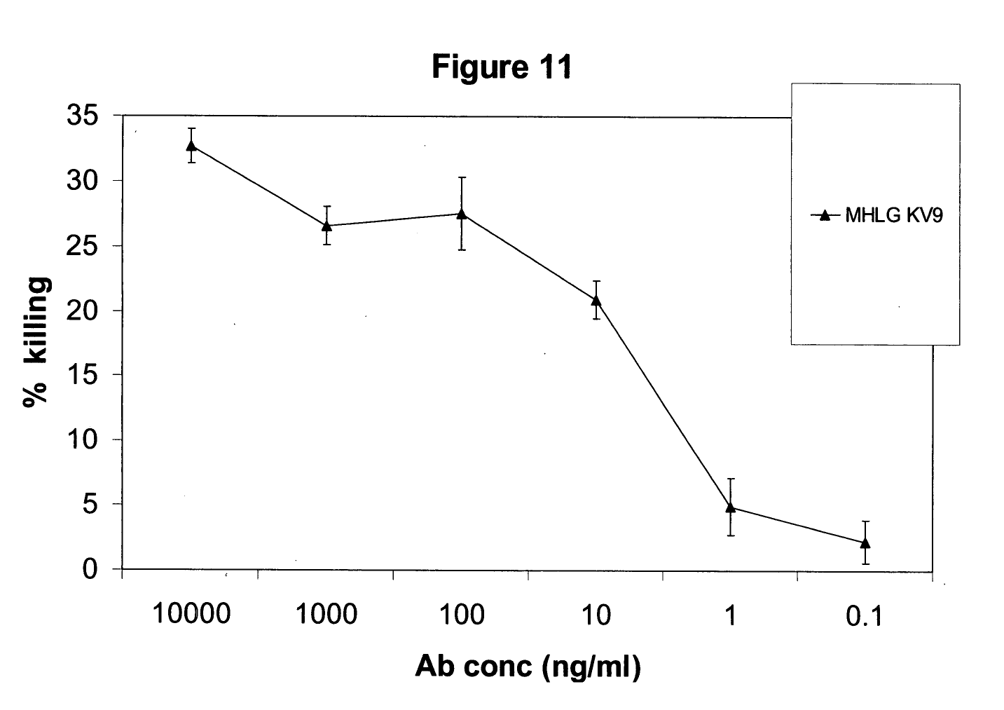

FIG. 6 shows the efficacy of the humanized M-HLG/M-KV9 construct of the 225.28S antibody in antibody mediated cell killing via human PBMC cells. Target cells are human A2058 cells, and one can see a strong increase in antibody mediated cell killing. The same effect can be observed when using human smooth muscle cells as target cells. These cells are primary cells and not derived from a tumor. They serve as a model for the targeting of pericytes, since those smooth muscle cells are a kind of precursor cell for the pericytes in neovasculature. One difference is noteworthy between these to experiments. The melanoma cells showed a higher degree of resistance towards PBMC induced killing than the smooth muscle cells. For this, incubation of the targets with the antibody and the effectors was 24 h, whereas for the smooth muscle cells approximately the same killing was achieved within 4 h. The antibody mediated cell killing of the smooth muscle cells is shown in FIG. 11.

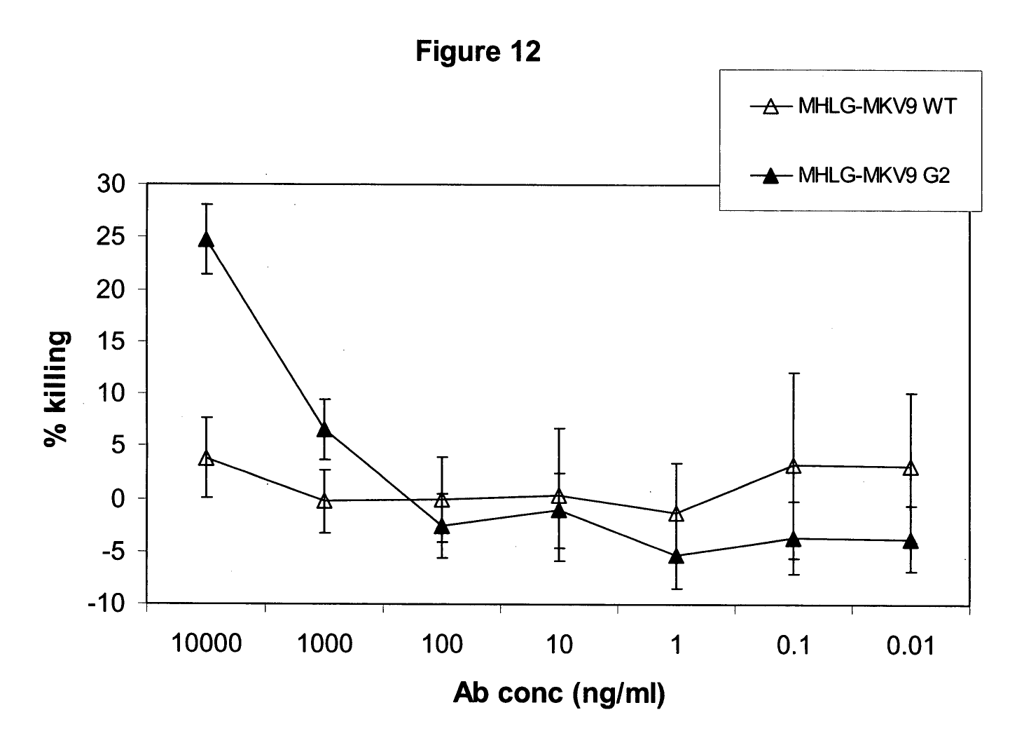

For the glioma cell-line LN229, it could be clearly shown (FIG. 12) that glycoengineering of the humanized anti-MCSP antibody could strongly increase its potency in antibody mediated cellular cytotoxicity (ADCC). The unmodified antibody showed hardly any activity, whereas the G2 version of the same antibody gave rise to a significant level of target cell killing. This demonstrates that glycoengineering can enhance the potency of antibodies that previously show unsatisfactory activity.

1. An isolated polynucleotide comprising:
a. a sequence selected from the group consisting of: SEQ ID NO:61; SEQ ID NO:63; SEQ ID NO:65; SEQ ID NO:67; SEQ ID NO:69; SEQ ID NO:71; SEQ ID NO:73; SEQ ID NO:75; and
b. a sequence selected from the group consisting of: SEQ ID NO:77; SEQ ID NO:79; SEQ ID NO:81; SEQ ID NO:83; SEQ ID NO:85; SEQ ID NO:87; SEQ ID NO:89; SEQ ID NO:91; and SEQ ID NO:93; and
c. SEQ ID NO:95.

2. An isolated polynucleotide comprising
a. a sequence selected from the group consisting of: SEQ ID NO:97; SEQ ID NO:99; and SEQ ID NO:101; and
b. SEQ ID NO:103 or SEQ ID NO:105; and
c. SEQ ID NO:107.

3. An isolated polynucleotide according to claim 1 or claim 2, which encodes a fusion polypeptide.

4. An isolated polynucleotide comprising a sequence selected from the group consisting of: SEQ ID No:3; SEQ ID No:5; SEQ ID No:7; SEQ ID No:9; SEQ ID No:11; SEQ ID No: 13; SEQ ID No:15; SEQ ID No:17; SEQ ID No:19; SEQ ID No:21; and SEQ ID No:23.

5-7. (canceled)

8. An isolated polynucleotide comprising a sequence selected from the group consisting of SEQ ID No:29, SEQ ID No:31, and SEQ ID No:33; SEQ ID NO:35; SEQ ID NO:37; SEQ ID NO:39; SEQ ID NO:41; SEQ ID NO:43; SEQ ID NO:45; SEQ ID NO:47; SEQ ID NO:49; SEQ ID NO:51.

9. (canceled)

10. An isolated polynucleotide according to claim 4 or 8, wherein said isolated polynucleotide encodes a fusion polypeptide.

11. (canceled)

12. An isolated polynucleotide comprising a sequence having at least 80% identity to a sequence selected from the group consisting of: SEQ ID No:1; SEQ ID No:3; SEQ ID No:5; SEQ ID No:7; SEQ ID No:9; SEQ ID No:11; SEQ ID No:13; SEQ ID No:15; SEQ ID No:17; SEQ ID No:19; SEQ ID No:21; and SEQ ID No:23, wherein said isolated polynucleotide encodes a fusion polypeptide.

13. An isolated polynucleotide comprising a sequence having at least 80% identity to a sequence selected from the group consisting of: SEQ ID NO:27, SEQ ID NO:29, SEQ ID NO:31; SEQ ID NO:33; SEQ ID NO:35; SEQ ID NO:37; SEQ ID NO:39; SEQ ID NO:41; SEQ ID NO:43; SEQ ID NO:45; SEQ ID NO:47; SEQ ID NO:49; and SEQ ID NO:51, wherein said isolated polynucleotide encodes a fusion polypeptide.

14-23. (canceled)

24. An isolated polynucleotide encoding a polypeptide having a sequence selected from the group consisting of SEQ ID No:4; SEQ ID No:6; SEQ ID No:8; SEQ ID No:10; SEQ ID No:12; SEQ ID No:14; SEQ ID No:16; SEQ ID No:18; SEQ ID No:20; SEQ ID No:22; and SEQ ID No:24.

25. An isolated polynucleotide encoding a polypeptide having a sequence selected from the group consisting of SEQ ID NO:30, SEQ ID NO:32; SEQ ID NO:34; SEQ ID NO:36; SEQ ID NO:38; SEQ ID NO:40; SEQ ID NO:42; SEQ ID NO:46; SEQ ID NO:48; and SEQ ID NO:52.

26. An expression vector comprising an isolated polynucleotide according to claim 1.

27-30. (canceled)

31. A host cell comprising an isolated polynucleotide according to claim 1.

32-34. (canceled)

35. A fusion polypeptide comprising a sequence selected from the group consisting of SEQ ID No:2; SEQ ID No:4; SEQ ID No:6; SEQ ID No:8; SEQ ID No:10; SEQ ID No:12; SEQ ID No:14; SEQ ID No:16; SEQ ID No:18; SEQ ID No:20; SEQ ID No:22; and SEQ ID No:24, or a variant thereof.

36. A fusion polypeptide comprising a sequence selected from the group consisting of: SEQ ID NO:28; SEQ ID NO:30, SEQ ID NO:32; SEQ ID NO:34; SEQ ID NO:36; SEQ ID NO:38; SEQ ID NO:40; SEQ ID NO:42; SEQ ID NO:46; SEQ ID NO:48; and SEQ ID NO:52. or a variant thereof.

37. An antigen binding molecule comprising the fusion polypeptide of claim 35.

38-39. (canceled)

40. The antigen binding molecule of claim 37, wherein said antigen binding molecule is an antibody.

41-48. (canceled)

49. An antigen binding molecule according to claim 40, wherein said antigen binding molecule has been glycoengineered to have an Fc region with modified oligosaccharides.

50. An antigen binding molecule according to claim 49, wherein said Fc region has been modified to have a reduced number of fucose residues as compared to the nonglycoengineered antigen binding molecule.

51-53. (canceled)

54. An antigen binding molecule according to claim 49, wherein said glycoengineered antibody has an increased ratio of GlcNAc residues to fucose residues in the Fc region compared to the nonglycoengineered antigen binding molecule.

55-56. (canceled)

57. An antigen binding molecule according to claim 49, wherein at least 20% of the oligosacchardies in the Fc region are bisected, nonfucosylated.

58-65. (canceled)

66. An antigen binding molecule according to claim 49, wherein at least 50% of the oligosaccharides in the Fc region are nonfucosylated.

67-69. (canceled)

70. A method of producing an antigen binding molecule capable of competing with the murine 225.28S monoclonal antibody for binding to human MCSP, said method comprising
a) culturing the host cell of claim 31 under conditions allowing the expression of said polynucleotide encoding said antigen binding molecule; and
b) recovering said antigen binding molecule.

71-72. (canceled)

73. A pharmaceutical composition comprising an antigen binding molecule according to claim 37 and a pharmaceutically acceptable carrier.

74. (canceled)

75. A method for identifying cells expressing MCSP in a sample or a subject comprising administering to said sample or subject an antigen binding molecule according to claim 37.

76-77. (canceled)

78. A method of treating an MCSP-mediated cell proliferation disorder in a subject in need thereof comprising administering a therapeutically effective amount of a pharmaceutical composition of claim 73 to said subject.

79. (canceled)

80. A method according to claim 78, wherein said treatment comprises blocking MCSP-mediated interactions selected from the group consisting of: MCSP ligand binding, melanoma cell adhesion, pericyte activation, chemotactic responses to fibronectin, cell spreading on ECM proteins, FAK signal transduction and ERK signal transduction.

81-108. (canceled)

109. A method according to claim 78, wherein said disorder is selected from the group consisting of: melanoma, glioma, lobular breast cancer, acute leukemia, or a solid tumor inducing neovascularization of blood vessels.

110. An isolated polynucleotide comprising at least one complementarity determining region of the murine 225.28S monoclonal antibody, or a variant or truncated form thereof containing at least the specificity-determining residues for said complementarity determining region, wherein said isolated polynucleotide encodes a fusion polypeptide.

111. An isolated polynucleotide according to claim 110 comprising at least two complementarity determining regions of the murine 225.28S monoclonal antibody, or a variant or truncated form thereof containing at least the specificity-determining residues for said complementarity determining region.

112-115. (canceled)

116. An isolated polynucleotide according to claim 111, wherein said complementarity determining regions comprise at least one sequence selected from the group consisting of: SEQ ID NO:61; SEQ ID NO:63; SEQ ID NO:65; SEQ ID NO:67; SEQ ID NO:69; SEQ ID NO:71; SEQ ID NO:73; SEQ ID NO:75; SEQ ID NO:77; SEQ ID NO:79; SEQ ID NO:81; SEQ ID NO:83; SEQ ID NO:85; SEQ ID NO:87; SEQ ID NO:89; SEQ ID NO:91; SEQ ID NO:93; and SEQ ID NO:95; and at least one sequence selected from the group consisting of: SEQ ID NO: 97, SEQ ID NO: 99, SEQ ID NO: 101; SEQ ID NO: 103; SEQ ID NO:105; SEQ ID NO: 107, or variants or truncated forms of said sequences that contain at least the specificity-determining residues for each of said complementarity determining regions.

117-120. (canceled)

121. A method for producing an antigen binding molecule having modified oligosaccharides in a host cell, said method comprising:
a. culturing a host cell glycoengineered to express at least one nucleic acid encoding a polypeptide having β(1,4)-N-acetylglucosaminyltransferase III activity under conditions which permit the production of said antigen binding molecule, and which permit the modification of the oligosaccharides present on the Fc region of said antigen binding molecule; and
b. isolating said antigen binding molecule wherein said antigen binding molecule is capable of competing with the murine 225.28S monoclonal antibody for binding to MCSP and wherein said antigen binding molecule or fragment thereof is chimeric or humanized.

122. A method according to claim 121, wherein said modified oligosaccharides have a reduced proportion of fucose residues as compared to the oligosaccharides of the nonglycoengineered antigen binding molecule.

123-124. (canceled)

125. A method according to claim 121, wherein said recombinant antibody or fragment thereof produced by said host cell has an increased proportion of bisected, nonfucosylated oligosaccharides in the Fc region of said polypeptide as compared to the antigen binding molecule produced by the nonglycoengineered cell.

126-127. (canceled)

128. A method according to claim 125, wherein at least 20% of the oligosaccharides in the Fc region of said polypeptide are bisected, nonfucosylated.

129-130. (canceled)

131. An antigen binding molecule glycoengineered to have increased effector function produced by the method according to claim 121.

132. (canceled)

133. An antigen binding molecules engineered to have increased Fc receptor binding affinity produced by the method of claim 121.

134. (canceled)

135. An antigen binding molecule according to claim 131, wherein said increased effector function is increased Fc-mediated cellular cytotoxicity.

136-144. (canceled)

145. An antigen binding molecule produced by the method of claim 121, wherein said antigen binding molecule is an antibody fragment containing the Fc region and engineered to have increased effector function.

146. An antigen binding molecule produced by the method of claim 121, wherein said antigen binding molecule is a fusion protein that includes a polypeptide having a sequence selected from the group consisting of: SEQ ID No:2; SEQ ID No:4; SEQ ID No:6; SEQ ID No:8; SEQ ID No:10; SEQ ID No:12; SEQ ID No:14; SEQ ID No:16; SEQ ID No:18; SEQ ID No:20; SEQ ID No:22; and SEQ ID No:24; and a region equivalent to the Fc region of an immunoglobulin and engineered to have increased effector function.

147. An antigen binding molecule produced by the method of claim 121, wherein said antigen binding molecule is a fusion protein that includes a polypeptide having a sequence selected from the group consisting of SEQ ID NO:28, SEQ ID NO:30, SEQ ID NO:32, SEQ ID NO: 34, SEQ ID NO: 36; SEQ ID NO: 38, SEQ ID NO:40, SEQ ID NO: 42, SEQ ID NO: 44, SEQ ID NO:46, SEQ ID NO:48, SEQ ID NO:50, and SEQ ID N052, and a region equivalent to the Fc region of an immunoglobulin and engineered to have increased effector function.

148-151. (canceled)

152. A method according to claim 125, wherein at least 40% of the oligosaccharides in the Fc region of said polypeptide are bisected, nonfucosylated.

153-157. (canceled)

158. A method of inducing lysis of activated pericytes in tumor neovasculature in a subject in need thereof, comprising administering to said subject the antigen binding molecule according to claim 49.

159. (canceled)

160. A method according to claim 158, wherein said antigen binding molecule is coadministered with another anti-angiogenic agent.

161-174. (canceled)

175. A host cell glycoengineered to express at least one nucleic acid molecule encoding a first polypeptide selected from the group consisting of: a polypeptide having β(1,4)-N-acetylglucosaminyltransferase III activity; a polypeptide having a-mannosidase II activity, and a polypeptide having β-(1,4)-galactosyltransferase activity, in an amount sufficient to modify the oligosaccharides in the Fc region of a second polypeptide produced by said host cell, wherein said second polypeptide is an antigen-binding molecule according to claim 37.

176. A host cell according to claim 175, wherein said first polypeptide is a polypeptide having β(1,4)-N-acetylglucosaminyltransferase III activity.

177. A host cell according to claim 175, wherein said first polypeptide is a polypeptide having α-mannosidase II activity.

178-181. (canceled)

182. The host cell of claim 175, wherein said antigen binding molecule produced by said host cell exhibits increased effector function compared to the antigen binding molecule produced by the nonglycoengineered host cell.

183. The host cell of claim 175, wherein said antigen binding molecule produced by said host cell exhibits increased Fc receptor binding affinity compared to the antigen binding molecule produced by the nonglycoengineered host cell.

184. A host cell according to claim 182, wherein said increased effector function is increased Fc-mediated cellular cytotoxicity.

185-191. (canceled)

192. A host cell according to claim 183, wherein said Fc receptor is an Fcγ activating receptor.

193. A host cell according to claim 183, wherein said Fc receptor is FcγRIIIA receptor.

194. An isolated polynucleotide according to claim 24, said polynucleotide further comprising a sequence encoding a polypeptide having a sequence selected from the group consisting of: SEQ ID NO:28 SEQ ID NO:30, SEQ ID NO:32, SEQ ID NO:34 and SEQ ID NO:36; SEQ ID NO:38; SEQ ID NO:40; SEQ ID NO:42; SEQ ID NO:44; SEQ ID NO:46; SEQ ID NO:48; SEQ ID NO:50; SEQ ID NO:52.

195. An isolated polynucleotide according to claim 24, said polynucleotide further comprising a sequence encoding a polypeptide having the sequence of an antibody Fc region, or a fragment thereof, from a species other than a murine species.

196. An isolated polynucleotide according to claim 25, said polynucleotide further comprising a sequence encoding a polypeptide having the sequence of an antibody Fc region, or a fragment thereof, from a species other than a murine species.


SEQUENCE LISTING (110 sequences)

SEQ ID NO: 1 - DNA, 366 bp, Artificial Sequence
  VH 225.28S
caggtgaagctgcagcagtcaggagggggcttggtgcaacctggaggatccatgaaactctcctgtgttgtctctggattcactttcagtaattactggatgaactgggtccgccagtctccagagaaggggcttgagtggattgcagaaattagattgaaatccaataattttggaagatattatgcggagtctgtgaaagggaggttcaccatctcaagagatgattccaaaagtagtgcctacctgcaaatgatcaacctaagagctgaagatactggcatttattactgtaccagttatggtaactacgttgggcactattttgaccactggggccaagggaccacggtcaccgtctcgagt

SEQ ID NO: 2 - protein, 122 aa, Artificial Sequence
  225.28S VH
QVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 3 - DNA, 366 bp, Artificial Sequence
  M-HHA
gaagtgcagctggtggagtctggaggaggcttggtcaagcctggcgggtccctgcggctctcctgtgcagcctccggattcacatttagcaactattggatgaactgggtgcggcaggctcctggaaagggcctcgagtgggtgggagagatcagattgaaatccaataacttcggaagatattacgctgcaagcgtgaagggccggttcaccatcagcagagatgattccaagaacacgctgtacctgcagatgaacagcctgaagaccgaggatacggccgtgtattactgtaccacatacggcaactacgttgggcactacttcgaccactggggccaagggaccaccgtcaccgtctccagt

SEQ ID NO: 4 - protein, 122 aa, Artificial Sequence
  M-HHA
EVQLVESGGGLVKPGGSLRLSCAASGFTFSNYWMNWVRQAPGKGLEWVGEIRLKSNNFGRYYAASVKGRFTISRDDSKNTLYLQMNSLKTEDTAVYYCTTYGNYVGHYFDHWGQGTTVTVSS

SEQ ID NO: 5 - DNA, 366 bp, Artificial Sequence
  M-HHB
gaagtgcagctggtggagtctggaggaggcttggtcaagcctggcgggtccctgcggctctcctgtgcagcctccggattcacatttagcaactattggatgaactgggtgcggcaggctcctggaaagggcctcgagtgggtgggagagatcagattgaaatccaataacttcggaagatattacgctgagagcgtgaagggccggttcaccatcagcagagatgattccaagaacacgctgtacctgcagatgaacagcctgaagaccgaggatacggccgtgtattactgtacctcctacggcaactacgttgggcactacttcgaccactggggccaagggaccaccgtcaccgtctccagt

SEQ ID NO: 6 - protein, 122 aa, Artificial Sequence
  M-HHB
EVQLVESGGGLVKPGGSLRLSCAASGFTFSNYWMNWVRQAPGKGLEWVGEIRLKSNNFGRYYAESVKGRFTISRDDSKNTLYLQMNSLKTEDTAVYYCTSYGNYVGHYFDHWGQGTTVTVSS

SEQ ID NO: 7 - DNA, 366 bp, Artificial Sequence
  M-HHC
gaagtgcagctggtggagtctggaggaggcttggtcaagcctggcgggtccctgcggctctcctgtgcagcctccggattcacatttagcaactattggatgaactgggtgcggcaggctcctggaaagggcctcgagtgggtggccaacatcagattgaaatccaataacttcggaagatattacgctgagagcgtgaagggccggttcaccatcagcagagatgattccaagaacacgctgtacctgcagatgaacagcctgaagaccgaggatacggccgtgtattactgtacctcctacggcaactacgttgggcactacttcgaccactggggccaagggaccaccgtcaccgtctccagt

SEQ ID NO: 8 - protein, 122 aa, Artificial Sequence
  M-HHC
EVQLVESGGGLVKPGGSLRLSCAASGFTFSNYWMNWVRQAPGKGLEWVANIRLKSNNFGRYYAESVKGRFTISRDDSKNTLYLQMNSLKTEDTAVYYCTSYGNYVGHYFDHWGQGTTVTVSS

SEQ ID NO: 9 - DNA, 366 bp, Artificial Sequence
  M-HLA
caggtgcagctggtgcagtctggcgctgaggtgaagaagcctggcgcctcggtgaaggtctcctgcaaggcctccggatacacattcaccaactattggatgaactgggtgcgacaggctcctggacaagggctcgagtggatgggcgagatcagattgaaatccaataacttcggaagatattacgcacagaagtttcagggcagagtcacaatgacacgggacacgtccacttccaccgtctacatggagctgagcagcctgagatccgaggatacggccgtctactactgcgcaagatacggcaactacgttgggcactacttcgaccactggggccaagggaccaccgtcaccgtctccagt

SEQ ID NO: 10 - protein, 122 aa, Artificial Sequence
  M-HLA
QVQLVQSGAEVKKPGASVKVSCKASGYTFTNYWMNWVRQAPGQGLEWMGEIRLKSNNFGRYYAQKFQGRVTMTRDTSTSTVYMELSSLRSEDTAVYYCARYGNYVGHYFDHWGQGTTVTVSS

SEQ ID NO: 11 - DNA, 366 bp, Artificial Sequence
  M-HLB
caggtgcagctggtgcagtctggcgctgaggtgaagaagcctggcgcctcggtgaaggtctcctgcaaggcctccggatacacattcaccaactattggatgaactgggtgcgacaggctcctggacaagggctcgagtggatgggcgagatcagattgaaatccaataacttcggaagatattacgcacagaagtttcagggcagagtcacaatcacacgggacacgagcatgtccaccgcctacatggagctgagcagcctgagatccgaggatacggccgtctactactgcgcagcctacggcaactacgttgggcactacttcgaccactggggccaagggaccaccgtcaccgtctccagt

SEQ ID NO: 12 - protein, 122 aa, Artificial Sequence
  M-HLB
QVQLVQSGAEVKKPGASVKVSCKASGYTFTNYWMNWVRQAPGQGLEWMGEIRLKSNNFGRYYAQKFQGRVTITRDTSMSTAYMELSSLRSEDTAVYYCAAYGNYVGHYFDHWGQGTTVTVSS

SEQ ID NO: 13 - DNA, 366 bp, Artificial Sequence
  M-HLC
caggtgcagctggtgcagtctggcgctgaggtgaagaagcctggcgcctcggtgaaggtctcctgcaaggcctccggatacacattcaccaactattggatgaactgggtgcgacaggctcctggacaagggctcgagtggatgggcgagatcagattgaaatccaataacttcggaagatactacgcagagtccgtgaagggcagagtcacaatcacacgggacacgagcatgtccaccgcctacatggagctgagcagcctgagatccgaggatacggccgtctactactgcgcagcctacggcaactacgttgggcactacttcgaccactggggccaagggaccaccgtcaccgtctccagt

SEQ ID NO: 14 - protein, 122 aa, Artificial Sequence
  M-HLC
QVQLVQSGAEVKKPGASVKVSCKASGYTFTNYWMNWVRQAPGQGLEWMGEIRLKSNNFGRYYAESVKGRVTITRDTSMSTAYMELSSLRSEDTAVYYCAAYGNYVGHYFDHWGQGTTVTVSS

SEQ ID NO: 15 - DNA, 366 bp, Artificial Sequence
  M-HLD
caggtgcagctggtgcagtctggcgctgaggtgaagaagcctggcgcctcggtgaaggtctcctgcaaggcctccggattcacattcagcaactattggatgaactgggtgcgacaggctcctggacaagggctcgagtggatgggcgagatcagattgaaatccaataacttcggaagatactacgcagagtccgtgaagggcagagtcacaatcacacgggacacgagcatgtccaccgcctacatggagctgagcagcctgagatccgaggatacggccgtctactactgcgcagcctacggcaactacgttgggcactacttcgaccactggggccaagggaccaccgtcaccgtctccagt

SEQ ID NO: 16 - protein, 122 aa, Artificial Sequence
  M-HLD
QVQLVQSGAEVKKPGASVKVSCKASGFTFSNYWMNWVRQAPGQGLEWMGEIRLKSNNFGRYYAESVKGRVTITRDTSMSTAYMELSSLRSEDTAVYYCAAYGNYVGHYFDHWGQGTTVTVSS

SEQ ID NO: 17 - DNA, 366 bp, Artificial Sequence
  M-HLE1
gaagtgcagctggtggagtctggaggaggcttggtcaagcctggcgggtccctgcggctctcctgtgcagcctccggattcacatttagcaactattggatgaactgggtgcggcaggcaccaggaaagggactcgagtgggtgggcgaaatccggttgaaatccaataacttcggaagatactacgcacagaagttccaggagagagtcacaatcacacgggacatgagcacctccaccgcctacatggagctgagcagcctgagatccgaggatacggccgtctactactgcgcagcctacggcaactacgttgggcactacttcgaccactggggccaagggaccaccgtcaccgtctccagt

SEQ ID NO: 18 - protein, 122 aa, Artificial Sequence
  M-HLE1
EVQLVESGGGLVKPGGSLRLSCAASGFTFSNYWMNWVRQAPGKGLEWVGEIRLKSNNFGRYYAQKFQERVTITRDMSTSTAYMELSSLRSEDTAVYYCAAYGNYVGHYFDHWGQGTTVTVSS

SEQ ID NO: 19 - DNA, 366 bp, Artificial Sequence
  M-HLE2
gaagtgcagctggtggagtctggaggaggcttggtcaagcctggcgggtccctgcggctctcctgtgcagcctccggattcacatttagcaactattggatgaactgggtgcggcaggcaccaggaaagggactcgagtgggtgggcgaaatccggttgaaatccaataacttcggaagatactacgcagagtccgtgaagggaagagtcacaatcacacgggacatgagcacctccaccgcctacatggagctgagcagcctgagatccgaggatacggccgtctactactgcgcagcctacggcaactacgttgggcactacttcgaccactggggccaagggaccaccgtcaccgtctccagt

SEQ ID NO: 20 - protein, 122 aa, Artificial Sequence
  M-HLE2
EVQLVESGGGLVKPGGSLRLSCAASGFTFSNYWMNWVRQAPGKGLEWVGEIRLKSNNFGRYYAESVKGRVTITRDMSTSTAYMELSSLRSEDTAVYYCAAYGNYVGHYFDHWGQGTTVTVSS

SEQ ID NO: 21 - DNA, 366 bp, Artificial Sequence
  M-HLF
gaagtgcagctggtggagtctggaggaggcttggtccagcctggcgggtccctgcggctctcctgtgcagcctccggattcacatttagcagctattggatgagctgggtgcggcaggctcctggaaagggcctcgagtgggtggccgagatcagattgaaatccaataacttcggaagatattacgctgcaagcgtgaagggccggttcaccatcagcagagatgattccaagaacacgctgtacctgcagatgaacagcctgaagaccgaggatacggccgtgtattactgtaccacatacggcaactacgttgggcactacttcgaccactggggccaagggaccaccgtcaccgtctccagt

SEQ ID NO: 22 - protein, 122 aa, Artificial Sequence
  M-HLF
EVQLVESGGGLVQPGGSLRLSCAASGFTFSSYWMSWVRQAPGKGLEWVAEIRLKSNNFGRYYAASVKGRFTISRDDSKNTLYLQMNSLKTEDTAVYYCTTYGNYVGHYFDHWGQGTTVTVSS

SEQ ID NO: 23 - DNA, 366 bp, Artificial Sequence
  M-HLG
gaagtgcagctggtggagtctggaggaggcttggtccagcctggcgggtccctgcggctctcctgtgcagcctccggattcacatttagcaactattggatgaactgggtgcggcaggctcctggaaagggcctcgagtgggtggccgagatcagattgaaatccaataacttcggaagatattacgctgcaagcgtgaagggccggttcaccatcagcagagatgattccaagaacacgctgtacctgcagatgaacagcctgaagaccgaggatacggccgtgtattactgtaccacatacggcaactacgttgggcactacttcgaccactggggccaagggaccaccgtcaccgtctccagt

SEQ ID NO: 24 - protein, 122 aa, Artificial Sequence
  M-HLG
EVQLVESGGGLVQPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSS

SEQ ID NO: 25 - DNA, 56 bp, Artificial Sequence
  M-VH Signal Sequence
atggactggacctggaggatcctcttcttggtggcagcagccacaggagcccactc

SEQ ID NO: 26 - protein, 19 aa, Artificial Sequence
  M-VH Signal Sequence
MDWTWRILFLVAAATGAHS

SEQ ID NO: 27 - DNA, 321 bp, Artificial Sequence
  KV-225.28S
gatatcgagctcacccaatctccaaaattcatgtccacatcagtaggagacagggtcagcgtcacctgcaaggccagtcagaatgtggatactaatgtagcgtggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggcggcaccaagctggaaatcaaa

SEQ ID NO: 28 - protein, 107 aa, Artificial Sequence
  225.28S VL
DIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIK

SEQ ID NO: 29 - DNA, 330 bp, Artificial Sequence
  M-KV1
gatatccagttgacccagtctccatccttcctgtctgcatctgtgggcgaccgggtcaccatcacctgcagggccagtcagaatgtggatactaacttagcttggtaccagcagaagccagggaaagcacctaagctcctgatctattcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 30 - protein, 109 aa, Artificial Sequence
  M-KV1
DIQLTQSPSFLSASVGDRVTITCRASQNVDTNLAWYQQKPGKAPKLLIYSASYRYTGVPSRFSGSGSGTEFTLTISSLQPEDFATYYCQQYNSYPLTFGGGTKVEIKRT

SEQ ID NO: 31 - DNA, 330 bp, Artificial Sequence
  M-KV2
gatatccagttgacccagtctccatccttcctgtctgcatctgtgggcgaccgggtcaccatcacctgcaaggccagtcagaatgtggatactaacgtggcttggtaccagcagaagccagggaaagcacctgagctcctgatctattcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 32 - protein, 109 aa, Artificial Sequence
  M-KV2
DIQLTQSPSFLSASVGDRVTITCKASQNVDTNVAWYQQKPGKAPELLIYSASYRYTGVPSRFSGSGSGTEFTLTISSLQPEDFATYYCQQYNSYPLTFGGGTKVEIKRT

SEQ ID NO: 33 - DNA, 330 bp, Artificial Sequence
  M-KV3
gatatccagttgacccagtctccatccttcctgtctgcatctgtgggcgaccgggtcaccgtcacctgcaaggccagtcagaatgtggatactaacgtggcttggtaccagcagaagccagggaaagcacctgagcctcttctgttctcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 34 - protein, 109 aa, Artificial Sequence
  M-KV3
DIQLTQSPSFLSASVGDRVTVTCKASQNVDTNVAWYQQKPGKAPEPLLFSASYRYTGVPSRFSGSGSGTEFTLTISSLQPEDFATYYCQQYNSYPLTFGGGTKVEIKRT

SEQ ID NO: 35 - DNA, 330 bp, Artificial Sequence
  M-KV4
gatatccagttgacccagtctccatccttcctgtctgcatctgtgggcgaccgggtcaccatcacctgcaaggccagtcagaatgtggatactaacgtggcttggtaccagcagaagccagggaaagcacctgagcctcttctgttctcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 36 - protein, 109 aa, Artificial Sequence
  M-KV4
DIQLTQSPSFLSASVGDRVTITCKASQNVDTNVAWYQQKPGKAPEPLLFSASYRYTGVPSRFSGSGSGTEFTLTISSLQPEDFATYYCQQYNSYPLTFGGGTKVEIKRT

SEQ ID NO: 37 - DNA, 330 bp, Artificial Sequence
  M-KV5
gatatccagttgacccagtctccatccttcctgtctgcatctgtgggcgaccgggtcaccatcacctgcaaggccagtcagaatgtggatactaacgtggcttggtacctgcagaagcccgggcagtctcctcagctcctgatctattcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 38 - protein, 109 aa, Artificial Sequence
  M-KV5
DIQLTQSPSFLSASVGDRVTITCKASQNVDTNVAWYLQKPGQSPQLLIYSASYRYTGVPSRFSGSGSGTEFTLTISSLQPEDFATYYCQQYNSYPLTFGGGTKVEIKRT

SEQ ID NO: 39 - DNA, 330 bp, Artificial Sequence
  M-KV6
gatatccagttgacccagtctccatccttcctgtctgcatctgtgggcgaccgggtcaccatcacctgcaaggccagtcagaatgtggatactaacgtggcttggttccagcagaggcccgggcagtctcctcgacgactgatctattcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 40 - protein, 109 aa, Artificial Sequence
  M-KV6
DIQLTQSPSFLSASVGDRVTITCKASQNVDTNVAWFQQRPGQSPRRLIYSASYRYTGVPSRFSGSGSGTEFTLTISSLQPEDFATYYCQQYNSYPLTFGGGTKVEIKRT

SEQ ID NO: 41 - DNA, 330 bp, Artificial Sequence
  M-KV7
gatatccagttgacccagtctccatccttcctgtctgcatctgtgggcgaccgggtcaccatcacctgcaaggccagtcagaatgtggatactaacgtggcttggtaccagcagaagccagggaaagcacctaagcctctgatctattcggcatcctaccggtacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 42 - protein, 109 aa, Artificial Sequence
  M-KV7
DIQLTQSPSFLSASVGDRVTITCKASQNVDTNVAWYQQKPGKAPKPLIYSASYRYTGVPSRFSGSGSGTEFTLTISSLQPEDFATYYCQQYNSYPLTFGGGTKVEIKRT

SEQ ID NO: 43 - DNA, 330 bp, Artificial Sequence
  M-KV8
gatatccagttgacccagtctccatccttcctgtctgcatctgtgggcgaccgggtcaccatcacctgcaaggccagtcagaatgtggatactaacgtggcttggtaccagcagaagccagggaaagcacctaagcttctgatcttctcggcatcctaccgttacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 44 - protein, 109 aa, Artificial Sequence
  M-KV8
DIQLTQSPSFLSASVGDRVTITCKASQNVDTNVAWYQQKPGKAPKLLIFSASYRYTGVPSRFSGSGSGTEFTLTISSLQPEDFATYYCQQYNSYPLTFGGGTKVEIKRT

SEQ ID NO: 45 - DNA, 330 bp, Artificial Sequence
  M-KV9
gatatccagttgacccagtctccatccttcctgtctgcatctgtgggcgaccgggtcaccatcacctgcaaggccagtcagaatgtggatactaacgtggcttggtaccagcagaagccagggcaggcacctaggcctctgatctattcggcatcctaccggtacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacggtg

SEQ ID NO: 46 - protein, 109 aa, Artificial Sequence
  M-KV9
DIQLTQSPSFLSASVGDRVTITCKASQNVDTNVAWYQQKPGQAPRPLIYSASYRYTGVPSRFSGSGSGTEFTLTISSLQPEDFATYYCQQYNSYPLTFGGGTKVEIKRT

SEQ ID NO: 47 - DNA, 327 bp, Artificial Sequence
  M-KV10
gatatccagttgacccagtctccatccttcctgtctgcatctgtgggcgaccgggtcaccatcacctgcagggccagtcagaatgtggatactaacttagcttggtaccagcagaagccagggcaggcacctaggcctctgatctattcggcatcctaccggtacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacg

SEQ ID NO: 48 - protein, 109 aa, Artificial Sequence
  M-KV10
DIQLTQSPSFLSASVGDRVTITCRASQNVDTNLAWYQQKPGQAPRPLIYSASYRYTGVPSRFSGSGSGTEFTLTISSLQPEDFATYYCQQYNSYPLTFGGGTKVEIKRT

SEQ ID NO: 49 - DNA, 327 bp, Artificial Sequence
  M-KV11
gatatccagttgacccagtctccatccttcctgtctgcatctgtgggcgaccgggtcaccatcacctgcagggccagtcagaatgtggatactaacgtggcttggtatcagcagaagccagggcaggcacctaggcctctgatctattcggcatcctaccggtacactggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacg

SEQ ID NO: 50 - protein, 109 aa, Artificial Sequence
  M-KV11
DIQLTQSPSFLSASVGDRVTITCRASQNVDTNVAWYQQKPGQAPRPLIYSASYRYTGVPSRFSGSGSGTEFTLTISSLQPEDFATYYCQQYNSYPLTFGGGTKVEIKRT

SEQ ID NO: 51 - DNA, 327 bp, Artificial Sequence
  M-KV12
gatatccagttgacccagtctccatccttcctgtctgcatctgtgggcgaccgggtcaccatcacctgcaaggccagtcagaatgtggatactaacgtggcttggtaccagcagaagccagggcaggcacctaggcctctgatctattcggcatcctacctgcagagcggcgtcccatcaaggttcagcggcagtggatccgggacagagttcactctcacaatctcaagcctgcaacctgaagatttcgcaacttactactgtcaacagtacaatagttaccctctgacgttcggcggaggtaccaaggtggagatcaagcgtacg

SEQ ID NO: 52 - protein, 109 aa, Artificial Sequence
  M-KV12
DIQLTQSPSFLSASVGDRVTITCKASQNVDTNVAWYQQKPGQAPRPLIYSASYLQSGVPSRFSGSGSGTEFTLTISSLQPEDFATYYCQQYNSYPLTFGGGTKVEIKRT

SEQ ID NO: 53 - DNA, 60 bp, Artificial Sequence
  M-VL Signal Sequence
atgagggtccccgctcagctcctgggcctcctgctgctctggttcccaggtgccaggtgt

SEQ ID NO: 54 - protein, 20 aa, Artificial Sequence
  M-VL Signal Sequence
MRVPAQLLGLLLLWFPGARC

SEQ ID NO: 55 - DNA, 318 bp, Artificial Sequence
  Constant-Light
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 56-60 - skipped (5 entries)

SEQ ID NO: 61 - DNA, 15 bp, Artificial Sequence
  Heavy Chain CDR1 MCSP Kabat
aattactggatgaac

SEQ ID NO: 62 - protein, 5 aa, Artificial Sequence
  Heavy Chain CDR1 MCSP Kabat
NYWMN

SEQ ID NO: 63 - DNA, 15 bp, Artificial Sequence
  Heavy Chain CDR1 MCSP Kabat
agctattggatgagc

SEQ ID NO: 64 - protein, 5 aa, Artificial Sequence
  Heavy Chain CDR1 MCSP Kabat
SYWMS

SEQ ID NO: 65 - DNA, 18 bp, Artificial Sequence
  Heavy Chain CDR1 MCSP Chothia
ggattcactttcagtaat

SEQ ID NO: 66 - protein, 6 aa, Artificial Sequence
  Heavy Chain CDR1 MCSP Chothia
GFTFSN

SEQ ID NO: 67 - DNA, 18 bp, Artificial Sequence
  Heavy Chain CDR1 MCSP Chothia
ggatacacattcaccaac

SEQ ID NO: 68 - protein, 6 aa, Artificial Sequence
  Heavy Chain CDR1 MCSP Chothia
GYTFTN

SEQ ID NO: 69 - DNA, 18 bp, Artificial Sequence
  Heavy Chain CDR1 MCSP Chothia
ggattcacatttagcagc

SEQ ID NO: 70 - protein, 6 aa, Artificial Sequence
  Heavy Chain CDR1 MCSP Chothia
GFTFSS

SEQ ID NO: 71 - DNA, 30 bp, Artificial Sequence
  Heavy Chain CDR1 MCSP AbM
ggattcactttcagtaattactggatgaac

SEQ ID NO: 72 - protein, 10 aa, Artificial Sequence
  Heavy Chain CDR1 MCSP AbM
GFTFSNYWMN

SEQ ID NO: 73 - DNA, 30 bp, Artificial Sequence
  Heavy Chain CDR1 MCSP AbM
ggatacacattcaccaactattggatgaac

SEQ ID NO: 74 - protein, 10 aa, Artificial Sequence
  Heavy Chain CDR1 MCSP AbM
GYTFTNYWMN

SEQ ID NO: 75 - DNA, 30 bp, Artificial Sequence
  Heavy Chain CDR1 MCSP AbM
ggattcacatttagcagctattggatgagc

SEQ ID NO: 76 - protein, 10 aa, Artificial Sequence
  Heavy Chain CDR1 MCSP AbM
GFTFSSYWMS

SEQ ID NO: 77 - DNA, 57 bp, Artificial Sequence
  Heavy Chain CDR2 MCSP Kabat
gaaattagattgaaatccaataattttggaagatattatgcggagtctgtgaaaggg

SEQ ID NO: 78 - protein, 19 aa, Artificial Sequence
  Heavy Chain CDR2 MCSP Kabat
EIRLKSNNFGRYYAESVKG

SEQ ID NO: 79 - DNA, 57 bp, Artificial Sequence
  Heavy Chain CDR2 MCSP Kabat
gagatcagattgaaatccaataacttcggaagatattacgctgcaagcgtgaagggc

SEQ ID NO: 80 - protein, 19 aa, Artificial Sequence
  Heavy Chain CDR2 MCSP Kabat
EIRLKSNNFGRYYAASVKG

SEQ ID NO: 81 - DNA, 57 bp, Artificial Sequence
  Heavy Chain CDR2 MCSP Kabat
aacatcagattgaaatccaataacttcggaagatattacgctgagagcgtgaagggc

SEQ ID NO: 82 - protein, 19 aa, Artificial Sequence
  Heavy Chain CDR2 MCSP Kabat
NIRLKSNNFGRYYAESVKG

SEQ ID NO: 83 - DNA, 57 bp, Artificial Sequence
  Heavy Chain CDR2 MCSP Kabat
gagatcagattgaaatccaataacttcggaagatattacgcacagaagtttcagggc

SEQ ID NO: 84 - protein, 19 aa, Artificial Sequence
  Heavy Chain CDR2 MCSP Kabat
EIRLKSNNFGRYYAQKFQG

SEQ ID NO: 85 - DNA, 57 bp, Artificial Sequence
  Heavy Chain CDR2 MCSP Kabat
gaaatccggttgaaatccaataacttcggaagatactacgcacagaagttccaggag

SEQ ID NO: 86 - protein, 19 aa, Artificial Sequence
  Heavy Chain CDR2 MCSP Kabat
EIRLKSNNFGRYYAQKFQE

SEQ ID NO: 87 - DNA, 57 bp, Artificial Sequence; identical to SEQ ID NO: 79
  Heavy Chain CDR2 MCSP Kabat

SEQ ID NO: 88 - protein, 19 aa, Artificial Sequence
  Heavy Chain CDR2 MCSP Kabat
EIRLKSNNFGRYYAASVKG

SEQ ID NO: 89 - DNA, 30 bp, Artificial Sequence
  Heavy Chain CDR2 MCSP Chothia
agattgaaatccaataattttggaagatat

SEQ ID NO: 90 - protein, 10 aa, Artificial Sequence
  Heavy Chain CDR2 MCSP Chothia
RLKSNNFGRY

SEQ ID NO: 91 - DNA, 36 bp, Artificial Sequence
  Heavy Chain CDR2 MCSP AbM
gaaattagattgaaatccaataattttggaagatat

SEQ ID NO: 92 - protein, 12 aa, Artificial Sequence
  Heavy Chain CDR2 MCSP AbM
EIRLKSNNFGRY

SEQ ID NO: 93 - DNA, 36 bp, Artificial Sequence
  Heavy Chain CDR2 MCSP AbM
aacatcagattgaaatccaataacttcggaagatat

SEQ ID NO: 94 - protein, 12 aa, Artificial Sequence
  Heavy Chain CDR2 MCSP AbM
NIRLKSNNFGRY

SEQ ID NO: 95 - DNA, 33 bp, Artificial Sequence
  Heavy Chain CDR3 MCSP Kabat, Chothia, AbM
tatggtaactacgttgggcactattttgaccac

SEQ ID NO: 96 - protein, 11 aa, Artificial Sequence
  Heavy Chain CDR3 MCSP Kabat, Chothia, AbM
YGNYVGHYFDH

SEQ ID NO: 97 - DNA, 33 bp, Artificial Sequence
  Kabat Light Chain CDR1 (MCSP)
aaggccagtcagaatgtggatactaatgtagcg

SEQ ID NO: 98 - protein, 11 aa, Artificial Sequence
  Kabat Light Chain CDR1 (MCSP)
KASQNVDTNVA

SEQ ID NO: 99 - DNA, 33 bp, Artificial Sequence
  Kabat Light Chain CDR1 (MCSP)
agggccagtcagaatgtggatactaacttagct

SEQ ID NO: 100 - protein, 11 aa, Artificial Sequence
  Kabat Light Chain CDR1 (MCSP)
RASQNVDTNLA

SEQ ID NO: 101 - DNA, 33 bp, Artificial Sequence
  Kabat Light Chain CDR1 (MCSP)
agggccagtcagaatgtggatactaacgtggct

SEQ ID NO: 102 - protein, 11 aa, Artificial Sequence
  Kabat Light Chain CDR1 (MCSP)
RASQNVDTNVA

SEQ ID NO: 103 - DNA, 21 bp, Artificial Sequence
  Kabat Light Chain CDR2(MCSP)
tcggcatcctaccgttacact

SEQ ID NO: 104 - protein, 7 aa, Artificial Sequence
  Kabat Light Chain CDR2(MCSP)
SASYRYT

SEQ ID NO: 105 - DNA, 21 bp, Artificial Sequence
  Kabat Light Chain CDR2(MCSP)
tcggcatcctacctgcagagc

SEQ ID NO: 106 - protein, 7 aa, Artificial Sequence
  Kabat Light Chain CDR2(MCSP)
SASYLQS

SEQ ID NO: 107 - DNA, 27 bp, Artificial Sequence
  Kabat Light Chain CDR3 (MCSP)
cagcaatataacagctatcctctgacg

SEQ ID NO: 108 - protein, 9 aa, Artificial Sequence
  Kabat Light Chain CDR3 (MCSP)
QQYNSYPLT

SEQ ID NO: 109 - protein, 328 aa, Artificial Sequence
  IgG1
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 987 bp, Artificial Sequence
  IgG1
accaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga